(19) United States
(12) Patent Application Publication
(10) Pub. No.: US 2024/0049986 A1
(43) Pub. Date: Feb. 15, 2024

(54) WEARABLE PHYSIOLOGICAL MONITORING DEVICE

(71) Applicant: Masimo Corporation, Irvine, CA (US)

(72) Inventors: Ammar Al-Ali, San Juan Capistrano, CA (US); Maxwell Gilmore, Irvine, CA (US); Stephen Scruggs, Newport Beach, CA (US); Valery G. Telfort, Irvine, CA (US); Chad A. DeJong, Los Angeles, CA (US); Steven Egge, Laguna Hills, CA (US)

(21) Appl. No.: 18/448,834

(22) Filed: Aug. 11, 2023

Related U.S. Application Data
(60) Provisional application No. 63/374,519, filed on Sep. 2, 2022.
(60) Provisional application No. 63/371,339, filed on Aug. 12, 2022.

(51) Int. Cl. A61B 5/11; A61B 5/00; A61B 5/01; A61B 5/33; A61B 5/287

(52) U.S. Cl. CPC A61B 5/1113; A61B 5/6833; A61B 5/7275; A61B 5/742; A61B 5/01; A61B 5/0008; A61B 5/33; A61B 5/287

(57) ABSTRACT

A wearable electronic monitoring device comprising one or more sensors configured to noninvasively measure one or more parameters of a user. The device can include a housing, a motion sensor positioned within the housing configured to generate one or more signals based on an orientation of the user, a display proximate a top portion of the housing that includes at least one display element responsive to an amount of health risk associated with the orientation of the user, one or more other sensors or user inputs, and one or more hardware processors configured to receive the one or more motion signals, determine the orientation of the user relative to a surface responsive to the one or more motion signals, determine the amount of health risk responsive to the orientation of the user, and change an appearance of the at least one display element responsive to the health risk.

### INCORPORATION BY REFERENCE TO ANY PRIORITY APPLICATIONS

The present application claims priority to U.S. Provisional Application No. 63/374,519, filed Sep. 2, 2022, and U.S. Provisional Application No. 63/371,339, filed Aug. 12, 2022. All of the above-listed applications and any and all other applications for which a foreign or domestic priority claim is identified in the Application Data Sheet as filed with the present application, are hereby incorporated by reference under 37 CFR 1.57.

### TECHNICAL FIELD

The present disclosure relates to the field of patient monitoring. More specifically, the disclosure describes, among other things, devices, methods, and/or systems for monitoring and/or displaying information regarding a patient's position, orientation, movement, and/or physiology.

### BACKGROUND

In clinical settings, such as hospitals, nursing homes, convalescent homes, skilled nursing facilities, post-surgical recovery centers, and the like, patients are frequently confined to a bed for extended periods of time. Sometimes the patients are unconscious or sedated to such an extent that they have limited ability to change or control their position and/or orientation in the bed. Such patients can be at risk of forming pressure ulcers, which pose a serious risk to the patient's health and well-being. Pressure ulcers, which may also be referred to as “bed sores,” “pressure sores,” and “decubitus ulcers,” involve injury to a patient's skin, and often the underlying tissue, which results from prolonged pressure forces applied to a site on the patient's body. Frequently, pressure ulcers develop on skin that covers bony areas of the body which have less muscle and/or fat tissue below the surface to distribute pressure applied thereto. Pressure ulcers can develop when such skin is subjected to prolonged contact with a surface of a bed or chair.

An established practice for patients at risk of forming pressure ulcers is to follow a turning protocol by which the patient is periodically repositioned, or “turned” to redistribute pressure forces placed on various points of the patient's body. Individuals at risk for a pressure ulcer are repositioned regularly. It is commonly suggested that patients be repositioned every 2 hours at specific inclination angles, and that the method of doing so minimizes the amount of friction and shear on the patient's skin. A repositioning log can be maintained and include key information, such as the time, body orientation, and outcome. Turning protocols, however, do not take into consideration position changes made by the patient between established turn intervals, which, in common practice, are neither observed nor recorded. Thus, it is possible that in some circumstances, the act of following a turn protocol can have an unintended negative clinical effect.

Care providers employ a variety of medical devices (for example, physiological sensors) that interact with patient monitoring devices which display a significant amount of patient health information. Such information is typically displayed on handheld monitoring devices or stationary monitoring devices with limited visual “real estate.” Often if not always, multiple patients are being monitored at once. Further, such health information is constantly fluctuating for multiple patients in a simultaneous manner, increasing the difficulty for a care provider to locate, evaluate, and respond to a particular piece of health information for a particular patient. Because care providers are under significant time pressure and only have a small amount of time to monitor, respond to, and/or treat individual patients under their care, it is incredibly difficult for care providers to quickly obtain information regarding a patient's orientation at any given time, let alone evaluate such information and determine if the patient's orientation needs to be adjusted. Even the slightest speed advantage for care providers in such situation can greatly reduce the likelihood that a patient will develop pressure ulcers and/or can enable care providers to provide potentially life-saving treatment.

### SUMMARY

Despite the importance of monitoring and making readily accessible to a care provider information regarding a patient's orientation, physiology, and/or risks associated with such information, commonly employed devices, methods, and/or systems for this purpose are lacking or non-existent. This disclosure describes, among other things, implementations of wearable devices, methods, and/or systems for monitoring, displaying, or otherwise indicating the orientation of a patient, physiological parameters of a patient, and/or risks associated with such patient information. Such wearable devices, methods, and/or systems can advantageously provide a care provider with actionable patient information in an easy to interpret fashion, ultimately to improve the standard of care provided to the patient. Such patient information can include orientation of a patient over time, risk associated with an orientation of a patient, temperature, cardiac activity and/or function, lung activity and/or function, and/or body sounds, among others. As an example, rather than relying on a patient monitor, which can be separated from the patient and/or can have connection issues with patient worn devices or systems, to assess the patient's orientation and/or risks associated with such patient information, the wearable devices described herein can provide such information directly from a wearable device that can be secured to the patient (e.g., secured to the patient's body). Such a wearable device can provide readily accessible patient information to a care provider and/or the patient themselves. Advantageously, the wearable devices described herein can perform these functions wirelessly, freeing the patient from being tethered by cabling. Furthermore, the devices, methods, and/or systems described herein can provide a patient (which can also be referred to herein as a “user”, “subject”, or “wearer”) a way to call for help, such as help from their care provider (which can also be referred to herein as a “caregiver”, “nurse”, or “doctor”) and/or a way to quickly contact their care provider for example, to request that the care provider attend to the patient. Some implementations of such devices can be configured to adhere to a surface, for example, of a hospital bed, so as to allow the device to be conveniently positioned relative to a patient.

Some implementations of the disclosed wearable devices (or portions of such devices) can be disposable, which can reduce the risk of cross-contamination between multiple users. Some implementations of the disclosed wearable devices (or portions of such devices) can be waterproof, thereby providing minimal disruption to ordinary activities of the user (for example, showering). Some implementations of the disclosed wearable devices include two separable components (which may also be referred to as “separate portions”). In such implementations, a first one of the components can be configured to secure to a portion of a user (for example, skin of the user) and a second one of the components can be configured to secure (for example, removably secure) to the first component. In some implementations, the first and second components are configured such that separation thereof is inhibited or prevented when the first component is secured to the user but is allowed when the first component is not secured to the user. Such implementations can be advantageous in scenarios where it is desirable to inhibit or prevent a user from interfering with operation of the wearable device. In some implementations, the wearable device includes a button configured to transition the wearable device (or a portion thereof such as the second component discussed above) between non-operational and operational modes. In some of such implementations, such button is inaccessible (for example, to the user wearing the wearable device and/or to another person, such as a care provider) unless the first and second components are separated from one another. Such implementation can advantageously prevent a user (for example, a child) from intentionally or unintentionally turning the wearable device off when the wearable device is secured to the user (which can ensure proper compliance in some situations).

Some implementations of the disclosed wearable devices are configured to monitor a user's orientation, position, and/or movement. For example, implementations of the disclosed wearable devices can be configured to monitor a user's orientation relative to a surface (such as a bed), movement in their environment (such as a number of steps taken and/or a type and/or quantity of exercise), a fall, and/or the like. Some implementations of wearable devices disclosed herein include a motion sensor, which can include an inertial motion unit and/or one or more accelerometers and/or one or more gyroscopes, and data from such motion sensor can be utilized to determine the user's orientation, position, and/or movement over time. Some implementations of the wearable devices disclosed herein can track the amount of time the user is in (and/or is not in) one or more of a plurality of orientations (for example, right side, left side, supine, among others) and illustrate the user's orientation history and/or trend in a display of the wearable device (which can be located on an exterior portion of the wearable device, for example). Furthermore, some implementations of the wearable devices disclosed herein can display or otherwise indicate a risk associated with an orientation of the user. Wearable devices described herein can be similar or identical to and/or incorporate any of the features described with respect to any of the devices, assemblies, methods, and/or systems described and/or illustrated in U.S. Pat. No. 11,406,286, filed Oct. 10, 2019, titled “PATIENT MONITORING DEVICE WITH IMPROVED USER INTERFACE,” in U.S. Pat Pub. No. US2023/0045000, filed Oct. 6, 2022, titled “PATIENT MONITORING DEVICE WITH IMPROVED USER INTERFACE,” in U.S. Pat App. No. 63/253,324, filed Oct. 7, 2021, titled “PATIENT MONITORING DEVICE WITH IMPROVED USER INTERFACE,” and in U.S. Pat Pub. No. US2021/0330200, filed Jul. 5, 2021, titled “SYSTEMS AND METHODS FOR PATIENT FALL DETECTION,” which are hereby incorporated by reference in their entirety and for all purposes.

Some implementations of the disclosed wearable devices include multiple temperature sensors operably positioned in different locations with respect to one another and with respect to the user's skin when in use. Such configurations can allow temperature to be determined at each of these different locations and compared with one another. In some implementations, thermal paths (which may be referred to as “thermal flow paths” or “heat flow paths”) between pairs of temperature sensors are defined by air and/or a thermally conductive element, which can provide additional information where thermal properties (for example, thermal conductivity values) are known. Differences between measurements at various ones of the temperature sensors can be utilized to provide more accurate estimates of body temperature (e.g., internal body temperature) of the user. Some implementations include two pairs of temperature sensors aligned with one another, where one of each pair is positioned farther from the user's skin/body (when the wearable device is in use) and the other one of each pair is positioned closer to the user's skin/body. Some implementations include an air gap (which can act as a thermal insulator) between one of such pairs and a thermally conductive element (for example, a metallic material) between the other one of such pairs. Temperature values determined based on each of the temperature sensors can be compared and utilized to approximate internal body temperature value(s) of the user. In various implementations, thermally conductive probe(s) can be utilized to transmit energy from a substrate of the wearable device (which can adhere to the user's skin) to and/or toward aligned temperature sensor(s). Wearable devices described herein can be similar or identical to and/or incorporate any of the features described with respect to any of the devices, assemblies, methods, and/or systems described and/or illustrated in U.S. Pat Pub. No. US2023/0087671, filed Sep. 20, 2022, titled “WEARABLE DEVICE FOR NONINVASIVE BODY TEMPERATURE MEASUREMENT,” which is hereby incorporated by reference in its entirety and for all purposes.

Some implementations of the disclosed wearable devices are configured to monitor body sounds of a user, which can include cardiac activity, lung activity, snoring, wheezing, coughing, choking, and/or breathing of the user and/or the like. For example, some implementations of the wearable devices disclosed herein include a diaphragm configured to move (for example, vibrate) responsive to body sounds (e.g., cardiac and/or lung activity) of a user to which the wearable device is attached, and such diaphragm movement (for example, vibration) can generate sound wave(s) within an interior portion of the wearable devices. Such sound wave(s) generated by the diaphragm can be detected by one or more microphones within or connected to such interior portion, and the microphone(s) can generate one or more signals based on the detected sound wave(s). Such one or more signals can, in turn, be received by one or more hardware processors of the wearable device for determination of such body sounds and/or related function (e.g., cardiac function and/or lung function) of the user. Wearable devices described herein can be similar or identical to and/or incorporate any of the features described with respect to any of the devices, assemblies, methods, and/or systems described and/or illustrated in U.S. Pat App. No. 61/547,007, filed Oct. 13, 2011, titled “PHYSIOLOGICAL ACOUSTIC MONITORING SYSTEM,” which is hereby incorporated by reference in its entirety and for all purposes.

Some implementations of the disclosed wearable devices are configured to monitor an electrocardiogram (ECG) activity of a user. For example, some implementations of the wearable devices disclosed herein include a plurality of ECG electrodes configured to output one or more signals responsive to a user's cardiac electrical activity. Such plurality of ECG electrodes can include one or more external electrodes and/or one or more internal electrodes. Such external electrodes can comprise a cable and an external ECG electrode configured to be secured to the user's body. Such one or more signals can, in turn, be received by one or more hardware processors of the wearable device for determination of an ECG of the user. Wearable devices described herein can be similar or identical to and/or incorporate any of the features described with respect to any of the devices, assemblies, methods, and/or systems described and/or illustrated in U.S. Pat. Pub. No. US2020/0329993, filed Apr. 16, 2020, titled “ELECTROCARDIOGRAM DEVICE,” in U.S. Pat. Pub. No. US2022/0233128, filed Apr. 4, 2022, titled “ELECTROCARDIOGRAM DEVICE,” and in U.S. Pat. App. No. 63/486,456, filed Feb. 2, 2023, titled “ELECTROCARDIOGRAM DEVICE,” which are hereby incorporated by reference in their entirety and for all purposes.

Disclosed herein is a self-contained adhesively and removably attached wearable electronic monitoring device. The device can comprise: a housing comprising an interior, a top portion, and a bottom portion, said bottom portion configured to face toward a user during monitoring of one or more physiological parameters of the user; a motion sensor positioned within an interior of the housing, said motion sensor configured to generate one or more signals based on an orientation of the user; a display proximate the top portion of the housing, the display including at least one display element responsive to an amount of health risk associated with the orientation of the user; one or more other sensors or user inputs; and one or more hardware processors positioned within the interior of the housing; wherein the one or more hardware processors are configured to: receive said one or more motion signals; determine the orientation of the user relative to a surface responsive to said one or more motion signals; determine the amount of health risk responsive to the orientation of the user; and change an appearance of the at least one display element responsive to the health risk.

In the above device or in other implementations as described herein, one or more of the following features can also be provided. In some implementations, said health risk is at least partially dependent upon an amount of time the user is in the orientation. In some implementations, said amount of time is not consecutive. In some implementations, the one or more hardware processors are further configured to: for each respective orientation of a plurality of orientations of the user with respect to the surface: increase a value of a timer associated with the respective orientation when the user is in the respective orientation; decrease the value of the timer when the user is not in the respective orientation; determine the amount of health risk for the respective orientation based at least in part on the value of the timer; and for each respective one of a plurality of display elements of the display: change an appearance of the respective one of the plurality of display elements based at least in part on the health risk associated with one of said plurality of orientations. In some implementations, a first one of said plurality of orientations is associated with a left side orientation of the user with respect to the surface; a second one of said plurality of orientations is associated with a right side orientation of the user with respect to the surface; and a third one of said plurality of orientations is associated with a supine orientation of the user with respect to the surface. In some implementations, for each respective one of the plurality of display elements of the display, the one or more hardware processors are further configured to: cause the appearance of the respective one of the plurality of display elements to have a first color when the health risk is greater than or equal to a threshold; and cause the appearance of the respective one of the plurality of portions to have a second color when the health risk is below the threshold, said second color being different than said first color. In some implementations, said plurality of orientations further comprises a plurality of orientations between said first one and said second one of said plurality of orientations including said third one. In some implementations, said health risk is associated with a combination of a plurality of factors. In some implementations, at least one of said factors is a physiological parameter of the user. In some implementations, said display comprises an arch shape. In some implementations, said display comprises a border having at least a first edge and a second edge; and each of said plurality of display elements of the display comprises a line or a region extending between the first and second edges of the border. In some implementations, said display only illustrates said health risk and does not include any other information. In some implementations, the device comprises a first portion configured to be attached to the user and a second portion configured to removably secure to the first portion, the second portion comprising said housing. In some implementations, the first portion comprises a frame and a substrate coupled to the frame, the substrate configured to be attached to the user.

In the above device or in other implementations as described herein, one or more of the following features can also be provided. In some implementations, the bottom portion of the housing comprises a first opening; the device further comprises: a diaphragm operably positioned proximate said first opening in said bottom portion, wherein, during monitoring, at least a portion of said diaphragm is configured to vibrate responsive to at least one of cardiac activity and lung activity of the user; a communication module positioned within the interior of the housing and configured to allow the device to wirelessly communicate with a separate device; and the one or more other sensors or user inputs comprise an audio transducer positioned within the interior of the housing and responsive to vibration of said diaphragm to output one or more transducer signals; wherein the one or more hardware processors are further configured to: receive said one or more transducer signals; determine at least one of a cardiac measurement and a lung measurement responsive to said one or more transducer signals; and wirelessly output to the separate device through the communication module data indicative of determined parameters of the user.

In the above device or in other implementations as described herein, one or more of the following features can also be provided. In some implementations, the one or more other sensors or user inputs comprise: a first temperature sensor and a second temperature sensor positioned within the interior of the housing, each of said first and second temperature sensors configured to generate one or more first temperature signals responsive to detected thermal energy, said first temperature sensor operably positioned to be closer to the user during monitoring than the second temperature sensor; and a third temperature sensor and a fourth temperature sensor positioned within the interior of the housing, each of said third and fourth temperature sensors configured to generate one or more second temperature signals responsive to detected thermal energy, said third temperature sensor operably positioned to be closer to the user during monitoring than the fourth temperature sensor; wherein the device further comprises: a second and a third opening in said bottom portion of the housing; a thermally conductive element comprising a portion positioned between the third and fourth temperature sensors; a first thermally conductive probe proximate the second opening of the housing and substantially aligned with the first temperature sensor; a second thermally conductive probe proximate the third opening of the housing and substantially aligned with the third temperature sensor; a communication module positioned within the interior of the housing and configured to allow the device to wirelessly communicate with a separate device; wherein the one or more hardware processors are further configured to: receive said first and second temperature signals; determine an indication of body temperature responsive to said first and second temperature signals; and wirelessly output to the separate device through the communication module the determined indication of body temperature.

In the above device or in other implementations as described herein, one or more of the following features can also be provided. In some implementations, the one or more other sensors or user inputs comprise: a user input proximate the top portion of the housing; and wherein the device further comprises: a communication module positioned within the interior of the housing and configured to allow the device to wirelessly communicate with a separate device; wherein the one or more hardware processors are further configured to: receive one or more user input signals responsive to said user input; and wirelessly output to the separate device through the communication module one or more communication signals based on said received one or more user input signals.

In the above device or in other implementations as described herein, one or more of the following features can also be provided. In some implementations, the device further comprises a plurality of cables and corresponding external ECG electrodes, said external ECG electrodes configured to attach to the user and output one or more signals responsive to the user's cardiac electrical activity; wherein the one or more hardware processors are further configured to: receive said one or more signals from said external ECG electrodes responsive to the user's cardiac electrical activity; and determine an ECG of the user responsive to said one or more signals. In some implementations, the device further comprises one or more internal ECG electrodes, said one or more internal ECG electrodes configured to output one or more signals responsive to the user's cardiac electrical activity;

For purposes of summarizing the disclosure, certain aspects, advantages, and novel features are discussed herein. It is to be understood that not necessarily all such aspects, advantages, or features will be embodied in any particular implementation of the disclosure, and an artisan would recognize from the disclosure herein a myriad of combinations of such aspects, advantages, or features.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIGS. 1A-1B illustrate top perspective views of a wearable device in accordance with aspects of this disclosure.

FIGS. 1C-1H illustrate top, bottom, side, and end views, respectively, of the wearable device of FIGS. 1A-1B in accordance with aspects of this disclosure.

FIG. 2 illustrates a schematic block diagram of certain features that may be included in the wearable device of FIGS. 1A-1B in accordance with aspects of this disclosure.

FIGS. 3A-3B illustrate top perspective views of a hub and a dock of the wearable device of FIGS. 1A-1B separated from one another in accordance with aspects of this disclosure.

FIGS. 4A-4B illustrate top perspective views of the dock of FIGS. 3A-3B in accordance with aspects of this disclosure.

FIG. 4C illustrates a side view of the dock of FIGS. 3A-3B in accordance with aspects of this disclosure.

FIG. 4D illustrates a bottom perspective view of the dock of FIGS. 3A-3B in accordance with aspects of this disclosure.

FIG. 4E illustrates an exploded view of the dock of FIGS. 3A-3B in accordance with aspects of this disclosure.

FIGS. 5A-5B illustrate top perspective views of the hub of FIGS. 3A-3B in accordance with aspects of this disclosure.

FIGS. 5C-5D illustrate top and bottom views, respectively, of the hub of FIGS. 3A-3B in accordance with aspects of this disclosure.

FIG. 5E illustrates a bottom perspective view of the hub of FIGS. 3A-3B in accordance with aspects of this disclosure.

FIG. 5F illustrates an end view of the hub of FIGS. 3A-3B in accordance with aspects of this disclosure.

FIGS. 5G-5H illustrate exploded views of the hub of FIGS. 3A-3B in accordance with aspects of this disclosure.

FIGS. 5I-5J illustrate bottom perspective views of a first portion of a housing of the hub of FIGS. 5G-5H in accordance with aspects of this disclosure.

FIGS. 5K-5M illustrate top and bottom perspective views of a second portion of the housing of the hub of FIGS. 5G-5H in accordance with aspects of this disclosure.

FIGS. 5N-5P illustrate top perspective views of, FIG. 5Q illustrates a bottom perspective view of, and FIG. 5R illustrates a bottom perspective partially exploded view of a third portion of the hub of FIGS. 5G-5H in accordance with aspects of this disclosure.

FIGS. 5S-5T illustrate top perspective views of portions of the hub of FIGS. 5G-5H in accordance with aspects of this disclosure.

FIG. 5U illustrates a top view of a circuit board of the hub of FIGS. 5G-5H in accordance with aspects of this disclosure.

FIG. 5V illustrates a top view of another circuit board of the hub of FIGS. 5G-5H in accordance with aspects of this disclosure.

FIG. 5W illustrates a cross-sectional view through the hub as identified in FIGS. 5C-5D in accordance with aspects of this disclosure.

FIG. 5X illustrates a cross-sectional view through the hub as identified in FIGS. 5C-5D in accordance with aspects of this disclosure.

FIG. 5Y illustrates a cross-sectional view through the wearable device of FIGS. 1A-1B as identified in FIG. 1D secured to a user's skin in accordance with aspects of this disclosure.

FIG. 5Z illustrates a cross-sectional view through the wearable device of FIGS. 1A-1B as identified in FIG. 1D secured to a user's skin in accordance with aspects of this disclosure.

FIG. 5AA illustrates an enlarged view of a portion of a wearable device having a display in accordance with aspects of this disclosure.

FIG. 6A illustrates a top perspective view of a wearable device in accordance with aspects of this disclosure.

FIGS. 6B-6G illustrate top, bottom, side, and end views, respectively, of the wearable device of FIG. 6A in accordance with aspects of this disclosure.

FIG. 7A illustrates a top perspective view of a hub and a dock of the wearable device of FIG. 6A separated from one another in accordance with aspects of this disclosure.

FIGS. 8A-8B illustrate top perspective views of the hub of FIG. 7A in accordance with aspects of this disclosure.

FIGS. 8C-8D illustrate top and bottom views, respectively, of the hub of FIG. 7A in accordance with aspects of this disclosure.

FIG. 8E illustrates a bottom perspective view of the hub of FIG. 7A in accordance with aspects of this disclosure.

FIG. 8F illustrates an end view of the hub of FIG. 7A in accordance with aspects of this disclosure.

FIGS. 8G-8H illustrate exploded views of the hub of FIG. 7A in accordance with aspects of this disclosure.

FIGS. 8I-8J illustrate bottom perspective views of a first portion of a housing of the hub of FIG. 8G-8H in accordance with aspects of this disclosure.

FIGS. 8K-8M illustrate top and bottom perspective views of a second portion of the housing of the hub of FIGS. 8G-8H in accordance with aspects of this disclosure.

FIGS. 8N-8P illustrate top perspective views of, FIG. 8Q illustrates a bottom perspective view of, and FIG. 8R illustrates a bottom perspective partially exploded view of a third portion of the hub of FIGS. 8G-8H in accordance with aspects of this disclosure.

FIGS. 8S-8T illustrate top perspective views of portions of the hub of FIGS. 8G-8H in accordance with aspects of this disclosure.

FIG. 8U illustrates a top view of a circuit board of the hub of FIGS. 8G-8H in accordance with aspects of this disclosure.

FIG. 8V illustrates a top view of another circuit board of the hub of FIGS. 8G-8H in accordance with aspects of this disclosure.

FIGS. 8W-8X illustrate top and bottom perspective partially exploded views, respectively, of a portion of the hub of FIGS. 8G-8H.

FIGS. 8Y-8AA illustrate top and bottom perspective views of a portion of the hub of FIGS. 8G-8H.

FIG. 8AB illustrates a cross-sectional view through the hub as identified in FIGS. 8C-8D in accordance with aspects of this disclosure.

FIG. 8AC illustrates a cross-sectional view through the hub as identified in FIGS. 8C-8D in accordance with aspects of this disclosure.

FIG. 8AD illustrates a cross-sectional view through the wearable device of FIG. 6A as identified in FIG. 6C secured to a user's skin in accordance with aspects of this disclosure.

FIG. 8AE illustrates a cross-sectional view through the wearable device of FIG. 6A as identified in FIG. 6C secured to a user's skin in accordance with aspects of this disclosure.

FIG. 9 illustrates a cross-sectional view of the wearable device of FIG. 6A secured to a user's skin in accordance with aspects of this disclosure.

FIG. 10 illustrates a cross-sectional view of a hub of the wearable device of FIG. 7A secured to a user's skin in accordance with aspects of this disclosure.

FIGS. 11A-11B illustrate top perspective views of wearable devices in accordance with aspects of this disclosure.

FIG. 12 illustrates care provider call devices in an example care environment in accordance with aspects of this disclosure.

FIGS. 13A-13E illustrate various views of a care provider call device in accordance with aspects of this disclosure.

FIG. 14 illustrates a schematic diagram of a care provider call device in accordance with aspects of this disclosure.

FIGS. 15A-15E illustrate various views of a care provider call device in accordance with aspects of this disclosure.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein.

Disclosed herein are wearable devices that can be used to measure, monitor, process, determine, display, and/or transmit (for example, wirelessly) one or more parameters of a user (which can also be referred to herein as a “subject”, “patient”, or “wearer”). The wearable devices disclosed herein can be self-contained adhesively and removably attached wearable electronic monitoring devices. The one or more parameters of the user can include an orientation, position, movement, and/or one or more physiological parameters of the user. Orientation, position, and/or movement of a user can include orientation of the user relative to a surface such as a bed, movement in their environment such as a number of steps taken and/or a type and/or a quantity of exercise, a fall, and/or the like. Physiological parameters of a user can include body temperature (e.g., internal body temperature), cardiac activity and/or function, lung activity and/or function, body sounds, and/or the like. The wearable devices disclosed herein can also include one or more user inputs (which can also be referred to herein as “user input devices”) that allow a user to contact, call, and/or alert a care provider (which can also be referred to herein as a “caregiver”, a “nurse”, or a “doctor”).

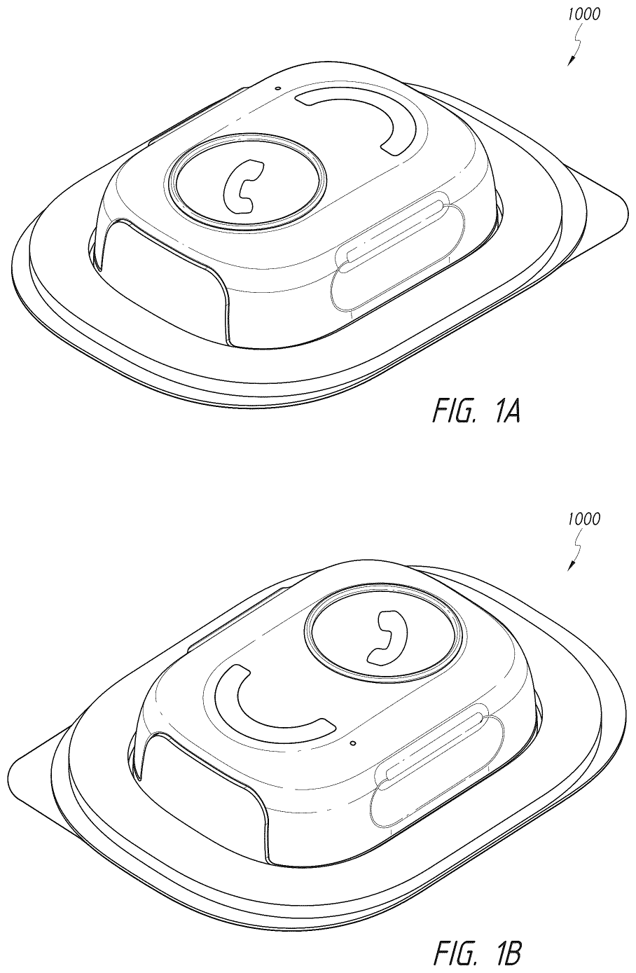

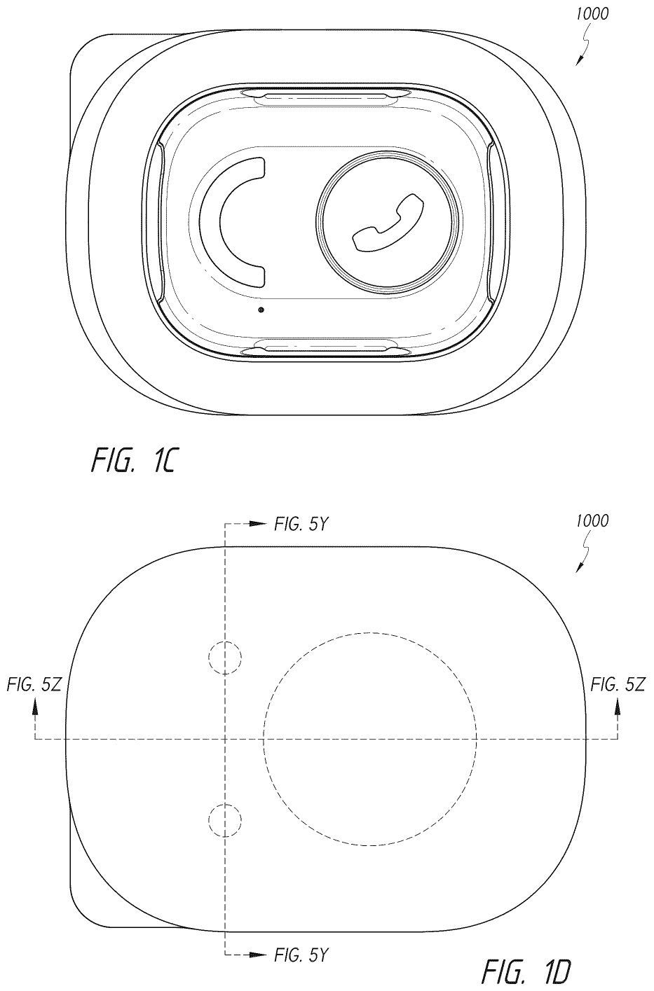

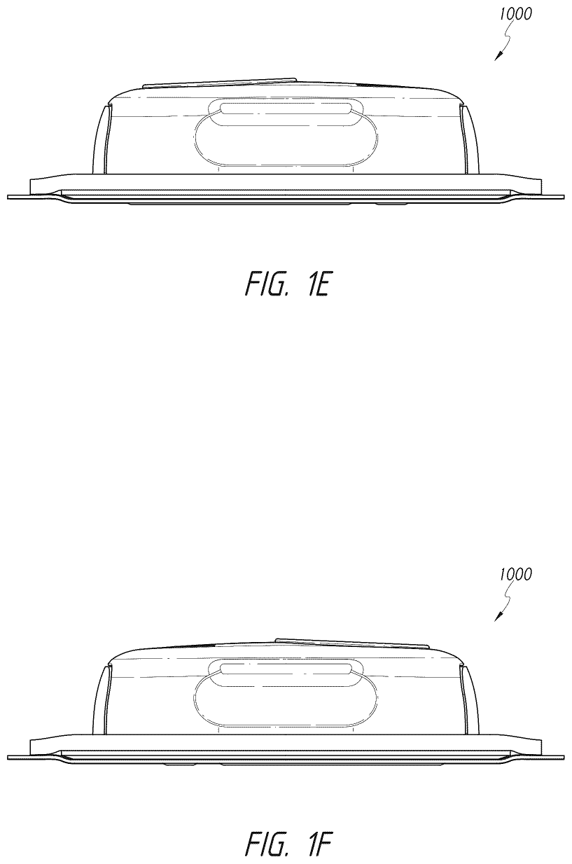

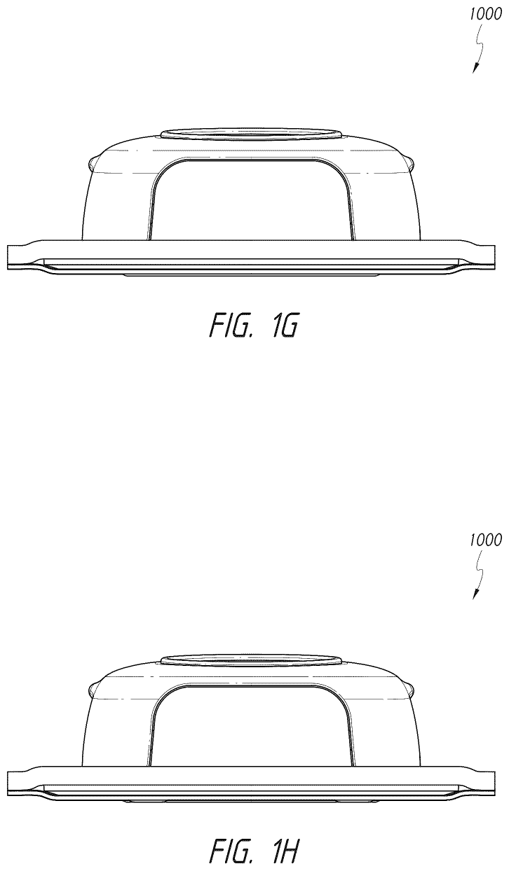

FIGS. 1A-1H illustrate various views of such a wearable device 1000. FIGS. 1A-1B illustrate top perspective views, FIG. 1C illustrates a top view, FIG. 1D illustrates a bottom view, FIGS. 1E-1F illustrate side views, and FIGS. 1G-1H illustrate end views of wearable device 1000. Wearable device 1000 can be configured to be secured to the skin of a user's body (e.g., to a portion of the user's body). For example, the wearable device 100 can be secured to a user's chest, such as over the user's manubrium, the broad upper portion of the sternum. In this position, the wearable device 1000 can be approximately centered relative to a longitudinal axis of the user's body and near the user's center of mass, a position that is useful in determining the user's orientation when, for example, the user is in a bed. Such a position can also advantageously position a display of the wearable device 1000, when included, in an easy to view location for the user's care provider and/or themselves. The wearable device 1000 can be secured to, affixed to or otherwise placed on various portions of the user's body in addition to or as an alternative to placement on the user's chest. For example, the wearable device 1000 can be secured to the user's back or more specifically, can be secured between a user's shoulder blades or on other portions of the user's back. Further examples of portions of a user's body that the wearable device 1000 can be secured to include the torso, arm, neck, head, leg, under the arm (e.g., armpit), among other portions of the user's body. The wearable device 1000 can secure (e.g., removably secure) to skin of a user and noninvasively measure, monitor, process, determine, display, and/or transmit (for example, wirelessly) orientation, position, movement, and/or one or more physiological parameters of a user using one or more sensors as described herein. The wearable device 1000 can also include one or more user inputs (e.g., a button) that allow a user to contact, call, and/or alert a care provider as described herein. Furthermore, the wearable device 1000 can wirelessly communicate with separate devices and/or systems (e.g., continuously or periodically wirelessly transmits physiological and/or other information of the user to a separate device and/or system).

The wearable device 1000 can be affixed to the user's skin using any form of medically-appropriate adherent material. For example, a portion of wearable device 1000 can include an adhesive material (e.g., a medical grade adhesive) that can allow the wearable device 1000 (or a portion thereof) to secure (e.g., removably secure) to the user's skin. As another example, the wearable device 1000 can include a pressure-sensitive adhesive that is coated or applied to a bottom surface of or a portion of the wearable device 1000 for securing the wearable device 1000 to the user's skin. In another example, the wearable device can be secured to a user's skin with an adhesive that wraps over the wearable device 1000 or at least a portion thereof. One skilled in the art will appreciate that many other materials and techniques can be used to affix the wearable device 1000 to the user without departing from the scope of the present disclosure.

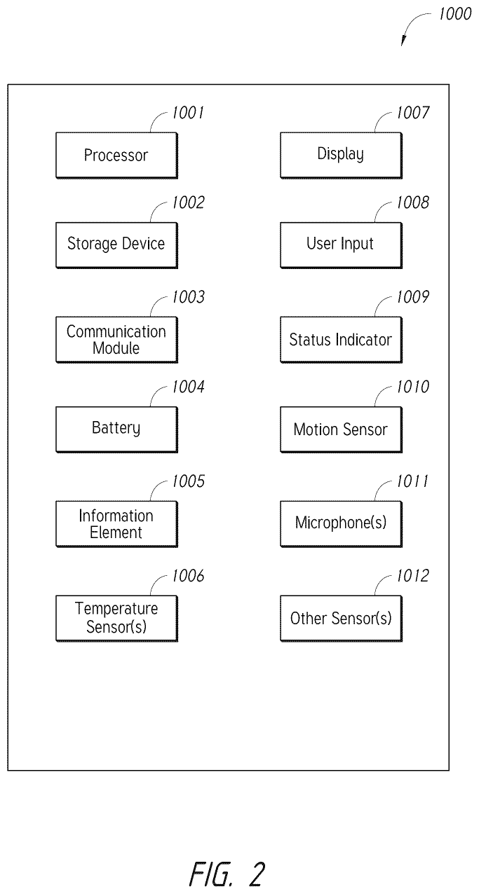

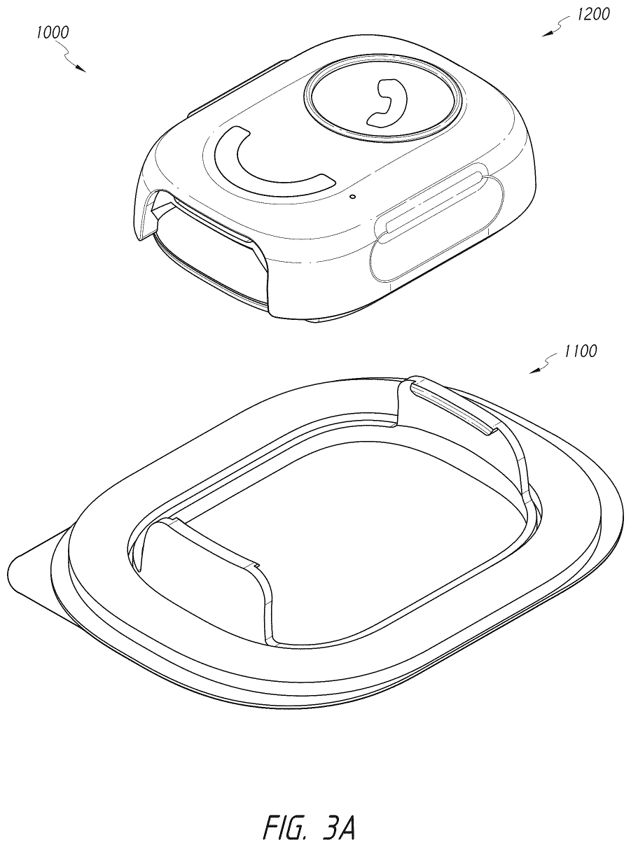

FIG. 2 illustrates an example schematic block diagram of wearable device 1000. Wearable device 1000 can include a hardware processor 1001, a storage device 1002, a communication module 1003, a battery 1004, an information element 1005, one or more temperature sensors 1006, a display 1007, a user input 1008, a status indicator 1009, a motion sensor 1010, one or more microphones (which can also be referred to herein as “audio transducer) 1011, and/or one or more other sensors 1012.

The processor 1001 can be configured, among other things, to process data, execute instructions to perform one or more functions, and/or control the operation of the wearable device 1000. For example, the processor 1001 can process physiological data and/or other data (for example, relating to motion/orientation) obtained from wearable device 1000 and can execute instructions to perform functions related to storing and/or transmitting such physiological data and/or other data. For example, the processor 1001 can process received data.

The storage device 1002 can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed physiological data and/or other data obtained from the wearable device 1000, for example.

The communication module 1003 can facilitate communication (via wired and/or wireless connection) between the wearable device 1000 (and/or components thereof) and separate devices, such as separate monitoring and/or mobile devices. For example, the communication module 1003 can be configured to allow the wearable device 1000 to wirelessly communicate with other devices, systems, and/or networks over any of a variety of communication protocols. The communication module 1003 can be configured to use any of a variety of wireless communication protocols, such as Wi-Fi (802.11x), Bluetooth®, ZigBee®, Zwave®, cellular telephony, infrared, near-field communications (NFC), RFID, satellite transmission, proprietary protocols, combinations of the same, and the like. The communication module 1003 can allow data and/or instructions to be transmitted and/or received to and/or from the wearable device 1000 and separate computing devices. The communication module 1003 can be configured to transmit (for example, wirelessly) processed and/or unprocessed physiological or other information to separate computing devices, which can include, among others, a mobile device (for example, an iOS or Android enabled smartphone, tablet, laptop), a desktop computer, a server or other computing or processing device for display and/or further processing, among other things. Such separate computing devices can be configured to store and/or further process the received physiological and/or other information, to display information indicative of or derived from the received information, and/or to transmit information—including displays, alarms, alerts, and notifications—to various other types of computing devices and/or systems that may be associated with a hospital, a care provider (for example, a primary care provider), and/or a designee (for example, an employer, a school, friends, family) that have permission to access the user's data. As another example, the communication module 1003 of the wearable device 1000 can be configured to wirelessly transmit processed and/or unprocessed obtained physiological information and/or other information (for example, motion, position, orientation, and/or location data) to a mobile phone which can include one or more hardware processors configured to execute an application that generates a graphical user interface displaying information representative of the processed or unprocessed physiological and/or other information obtained from the wearable device 1000. In some implementations, the communication module 1003 can transmit data to and/or receive data from a user's electronic medical record. In some implementations, the wearable device 1000 can be used for telehealth. For example, a user can be sent home with the wearable device 1000, which can transmit data to the cloud for a care provider to review. The communication module 1003 can be embodied in one or more components that are in communication with each other. The communication module 1003 can comprise a wireless transceiver, an antenna, and/or a near field communication (NFC) component, for example, NFC transponder 1233 discussed further below.

The battery 1004 can provide power for hardware components of the wearable device 1000 described herein. The battery 1004 can be, for example, battery 1232, described in more detail herein. The battery 1004 can be non-rechargeable. In such implementations, a battery life can be a week or more, two weeks or more, four weeks or more, two months or more, or more or less than these durations. In some implementations, the wearable device 1000 can include a removable battery isolator configured to electrically isolate the battery 1004 from other electronic components of the wearable device 1000 until a user or care provider desires to use the wearable device 1000. In some implementations, the battery 1004 can be rechargeable. For example, the battery 1004 can be a lithium, a lithium polymer, a lithium-ion, a lithium-ion polymer, a lead-acid, a nickel-cadmium, or a nickel-metal hydride battery. Additionally or alternatively, the wearable device 1000 can be configured to obtain power from a power source that is external to the wearable device 1000. For example, the wearable device 1000 can include or can be configured to connect to a cable which can itself connect to an external power source to provide power to the wearable device 1000. In implementations wherein the battery 1004 is rechargeable and/or the wearable device 1000 is configured to connect to a power cable, the wearable device 1000 (e.g., the hub 1200) can include a port for receiving such a power cable. Such a port can, for example, be positioned at a side, corner, or end of the wearable device (e.g., of a hub 1200 of the wearable device 1000 as described herein), and operably connect such an external power source to the battery 1004 and/or associated electronic components of the wearable device 1000. In some implementations, the wearable device 1000 is configured for induction charging and/or wireless charging.

The information element 1005 can be a memory storage element that stores, in non-volatile memory, information used to help maintain a standard of quality associated with the wearable device 1000. Illustratively, the information element 1005 can store information regarding whether the wearable device 1000 has been previously activated and whether the wearable device 1000 has been previously operational for a prolonged period of time, such as, for example, four hours, one day, two days, five days, ten days, twenty days. The information stored in the information element 1005 can be used to help detect improper re-use of the wearable device 1000, for example.

The wearable device 1000 can include one or more temperature sensor(s) 1006 that can continuously or periodically obtain temperature data of a user. Advantageously, in some implementations, the processor 1001 can compare temperature data from more than one temperature sensor 1006 to more accurately determine body temperature (e.g., internal body temperature) of the user. Each of the one or more temperature sensors 1006 can generate one or more signals responsive to detected thermal energy and such one or more signals can be received by processor 1001 for determination of body temperature value(s) of the user. Additionally or alternatively, each of the one or more temperature sensors 1006 can determine temperature values and transmit such temperature values to processor 1001 for determination of body temperature value(s). The one or more temperature sensors 1006 can be thermistors or integrated circuit (IC) temperature sensors, for example. The wearable device 1000 can incorporate temperature sensor(s), associated structure(s), and/or associated methods of user temperature determination similar or identical those described and/or illustrated in U.S. Pat Pub. No. US2023/0087671 incorporated by reference herein.

The wearable device 1000 can include a motion sensor 1010 configured to monitor motion and/or orientation of a user (e.g., over time). The motion sensor 1010 can include an inertial motion unit and/or one or more accelerometers and/or one or more gyroscopes. The motion sensor 1010 can generate one or more signals responsive to detected motion and/or orientation of the user. Such one or more signals can be received by the processor 1001 to determine the orientation of the user relative to a surface over time. Furthermore, the processor 1001 can determine an amount of risk associated with the orientation of the user. In an example of use, the wearable device 1000 can be worn by a user who has been determined to be at risk of forming one or more pressure ulcers, for example, a user who is confined to bed for an extended period of time. The wearable device 1000 can continuously or periodically (e.g., every second) monitor the orientation of the user to help determine whether the user is repositioned frequently enough to reduce the user's risk of forming a pressure ulcer.

In implementations wherein the motion sensor 1010 includes one or more accelerometers, measurements from such accelerometer(s) can be used by the wearable device 1000 (for example, by processor 1001) to determine motion and/or orientation of a user. The accelerometer(s) can measure and output signals related to a linear acceleration of the user with respect to gravity along three axes (for example, three, mutually orthogonal axes). For example, one axis, referred to as “roll,” can correspond to the longitudinal axis of and/or extending through the user's body (for example, along a length and/or height of the user). Accordingly, the roll reference measurement can be used to determine whether the user is in the prone position (for example, face down), the supine position (for example, face up), or on a side. Another reference axis of the accelerometer(s) is referred to as “pitch.” The pitch axis can correspond to the locations about the user's hip (for example, an axis extending between and/or through the user's hips). The pitch measurement can be used to determine whether the user is sitting up or lying down. A third reference axis of the accelerometer(s) is referred to as “yaw.” The yaw axis can correspond to a horizontal plane in which the user is located. When in bed, the user can be supported by a surface structure that generally fixes the user's orientation with respect to the yaw axis. Thus, in certain implementations, the yaw measurement is not used to determine the user's orientation when in a bed. The three axes that the accelerometer(s) can measure linear acceleration with respect to can be referred to as the “X,” “Y,” and “Z” axes.

The accelerometer(s) can provide acceleration information along three axes, and can provide acceleration information which is the equivalent of inertial acceleration minus local gravitational acceleration. The accelerometer(s) can be a micro-electromechanical system (MEMS), and can include piezo-resistors, among other forms of implementation. The accelerometer(s) can be high-impedance charge output or low-impedance charge output accelerometer(s) 1010. In some implementations, the accelerometer(s) may be tri-axial accelerometer(s), and the output of accelerometer(s) may include three signals, each of which represents measured acceleration along a particular axis. The output of the accelerometer(s) can be 8-bit, 12-bit, or any other appropriate-sized output signal. The outputs of the accelerometer(s) may be in analog or digital form. The accelerometer(s) can be used to determine the orientation and/or motion of the user to which the wearable device 1000 is attached.

In implementations wherein the motion sensor 1010 includes one or more gyroscope(s), such gyroscope(s) can be a three-axis digital gyroscope with angle resolution of two degrees and with a sensor drift adjustment capability of one degree. The term three-axis gyroscope as used herein includes its broad meaning known to a skilled artisan. The gyroscope(s) can provide outputs responsive to sensed angular velocity of the wearable device 1000 (as affixed to a user) with respect to three orthogonal axes corresponding to measurements of pitch, yaw, and roll (for example, see description provided above). A skilled artisan will appreciate that numerous other gyroscopes can be used in the wearable device 1000 without departing from the scope of the disclosure herein.

In certain implementations and discussed herein, one or more accelerometers and one or more gyroscopes can be integrated into a single hardware component which may be referred to as the motion sensor 1010 or an inertial measurement unit (IMU). In some implementations, such motion sensor 1010 or IMU can also include an embedded processor that handles, among other things, signal sampling, buffering, sensor calibration, and/or sensor fusion processing of the sensed inertial data. In other implementations, processor 1001 can perform these functions. And in still other implementations, sensed inertial data is minimally processed by the components of the wearable device 1000 and transmitted to an external device and/or system for further processing, thereby minimizing the complexity, power consumption, and/or cost of the wearable device 1000, all or a portion of which may be a single-use, disposable product.

Accelerometer(s) and/or gyroscope(s) of motion sensor 1010 can be similar or identical to any of the accelerometers and gyroscopes disclosed in U.S. Pat. No. 11,406,286 incorporated by reference herein. Wearable device 1000 (for example, via processor 1001) can be configured to determine orientation of a user (for example, relative to a bed or other surface) in a similar or identical manner as described with respect to any of the devices and/or systems disclosed in U.S. Pat. No. 11,406,286 incorporated by reference herein. Additionally or alternatively, wearable device 1000 (for example, via processor 1001) can be configured to determine orientation of a user and/or whether a user has fallen as described with respect to any of the devices and/or systems disclosed in U.S. Pat. Pub. No. 2021/0330200 incorporated by reference herein. Wearable device 1000 (for example, via processor 1001) can communicate with (for example, wirelessly transmit physiological data and/or other data) with a separate device, such as a patient monitor like any of those disclosed in U.S. Pat. No. 11,406,286 and/or U.S. Pat. Pub. No. 2021/0330200 incorporated by reference herein.

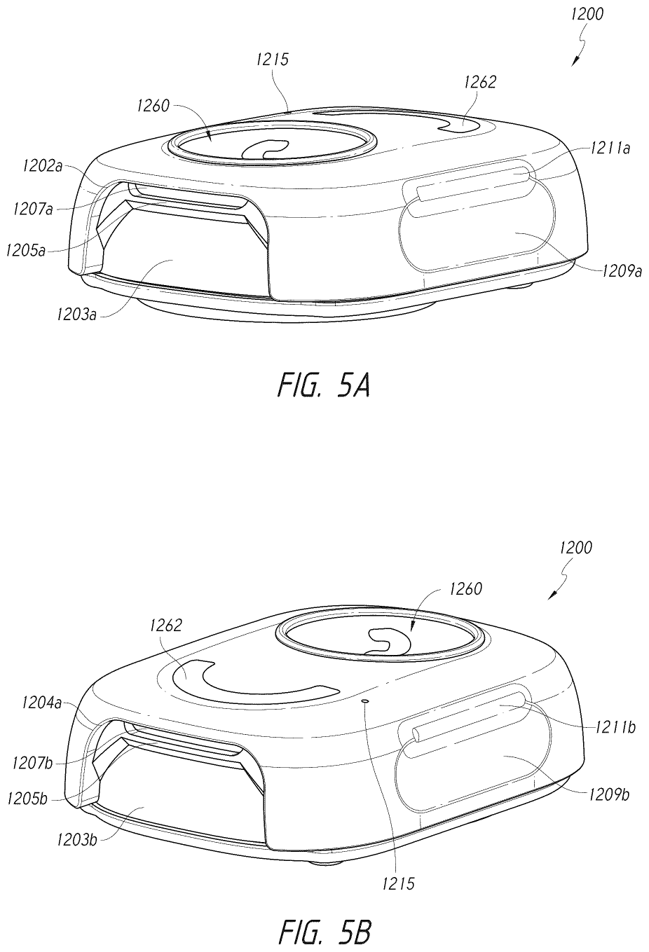

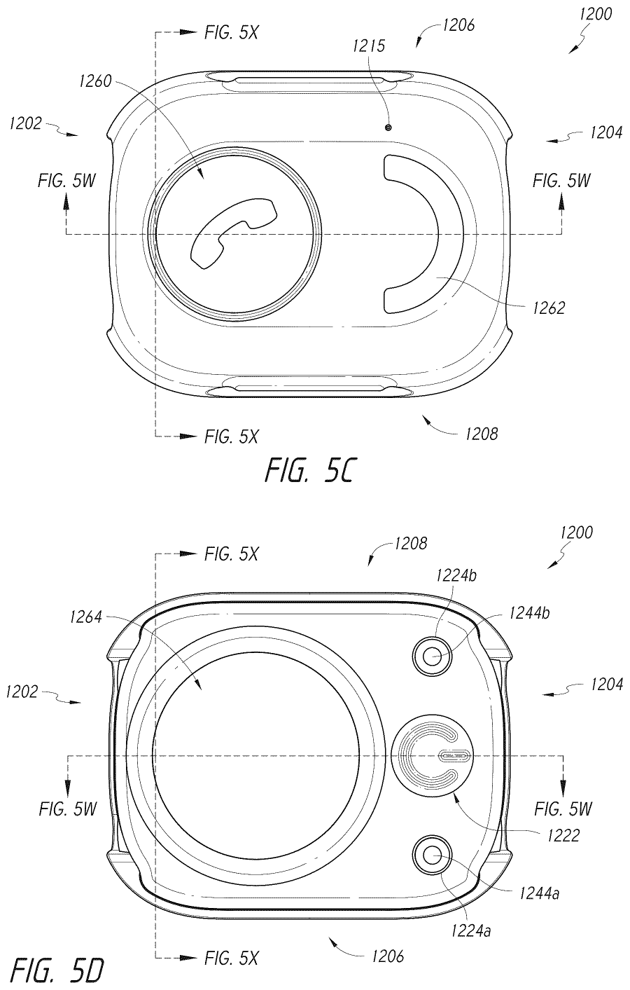

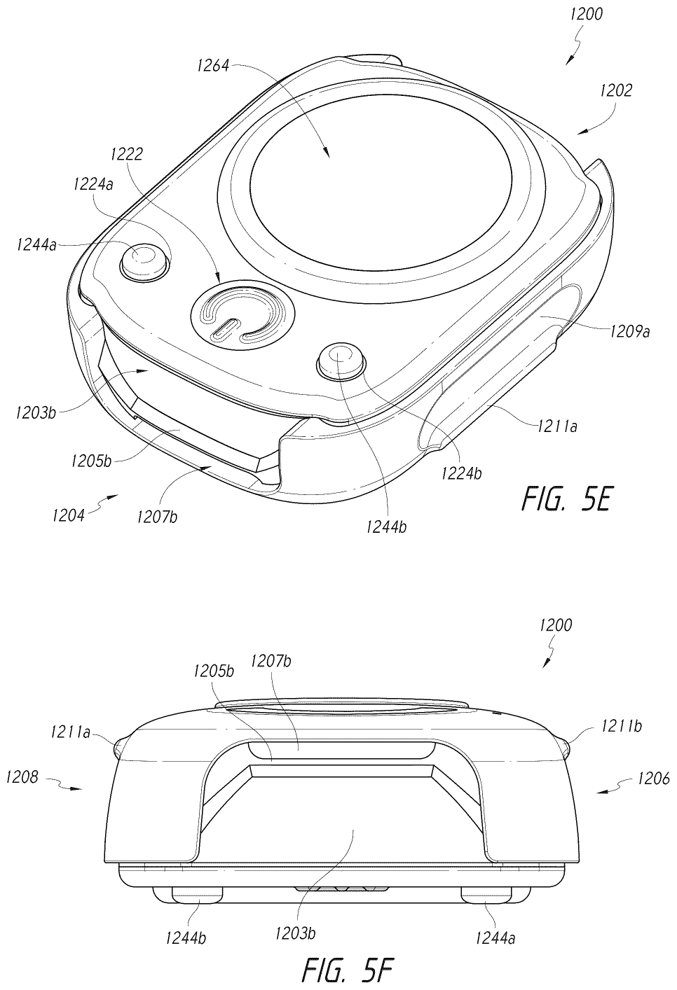

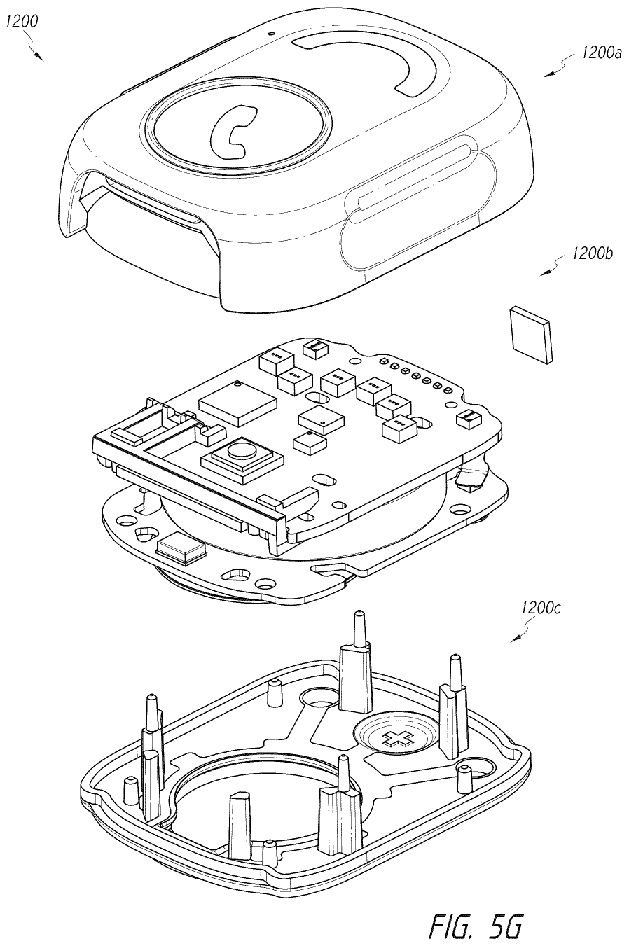

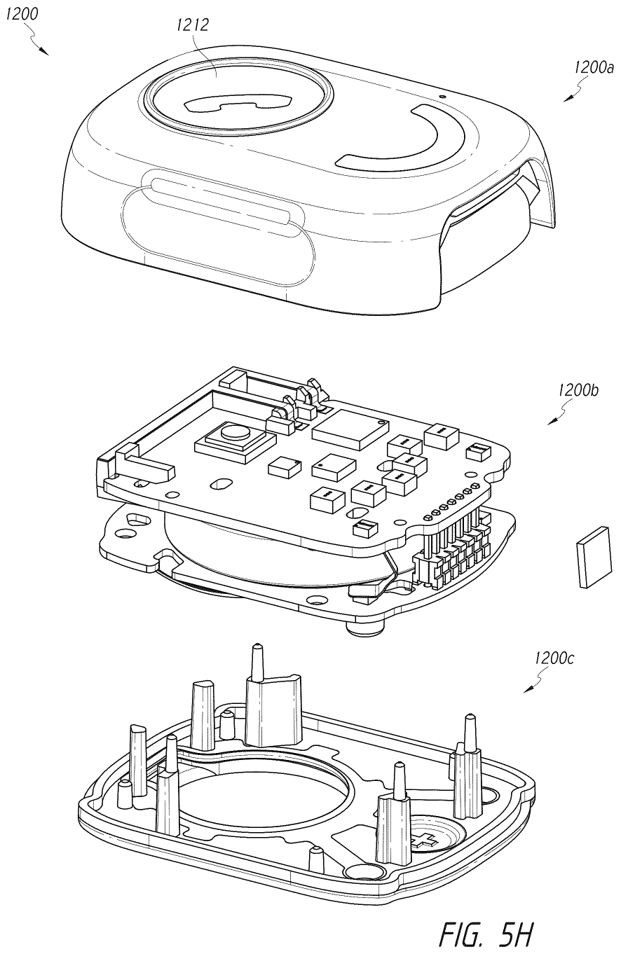

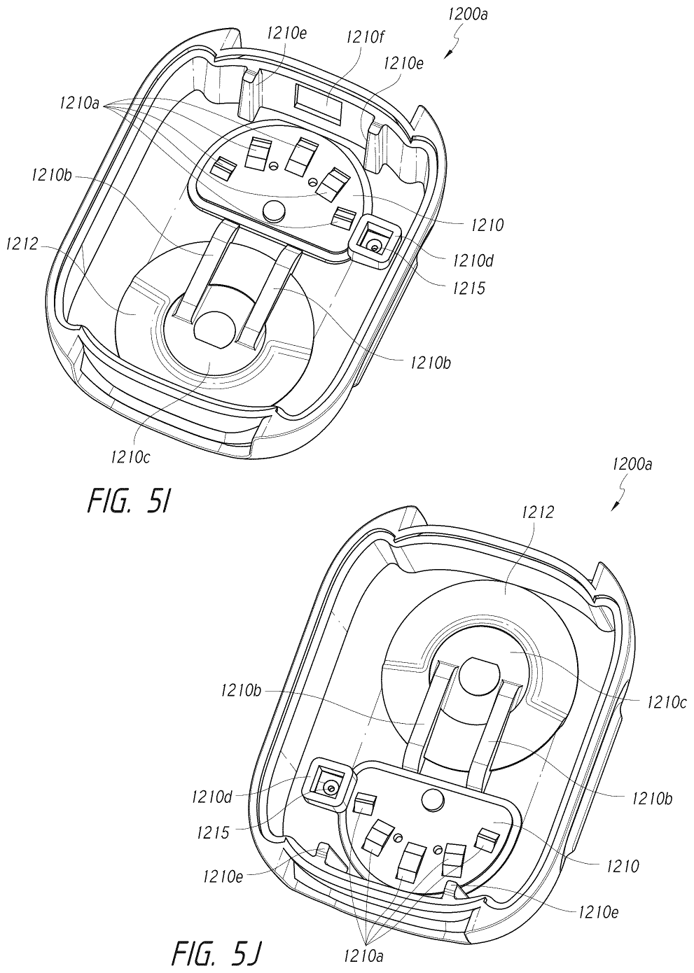

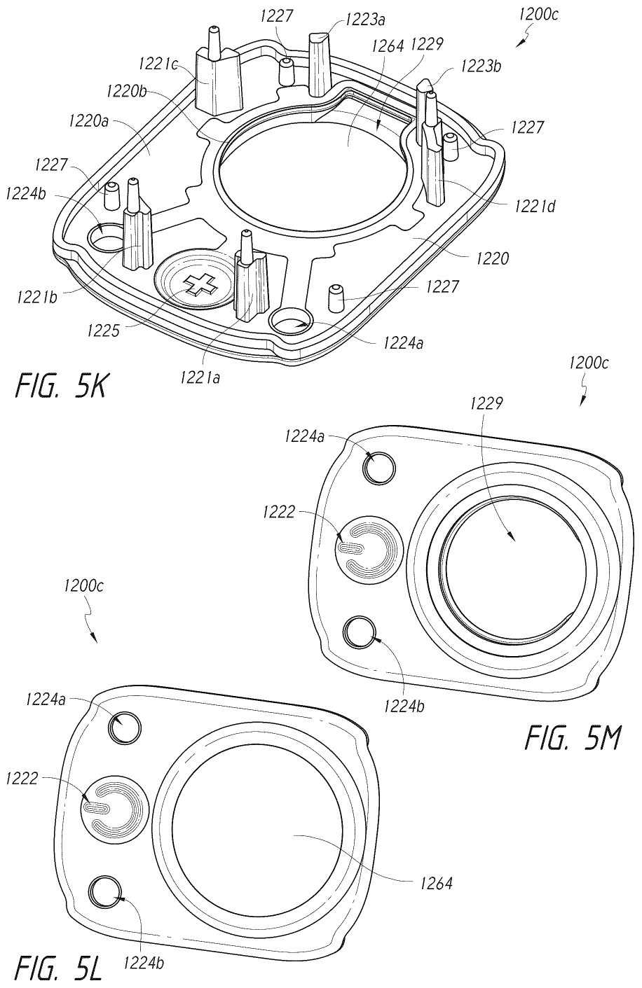

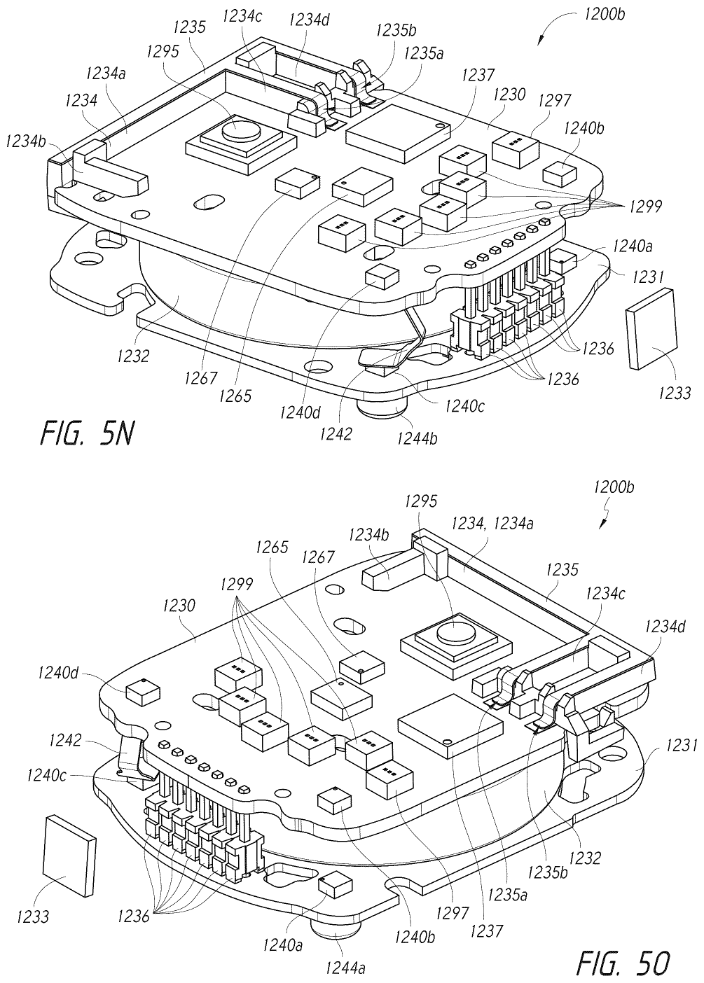

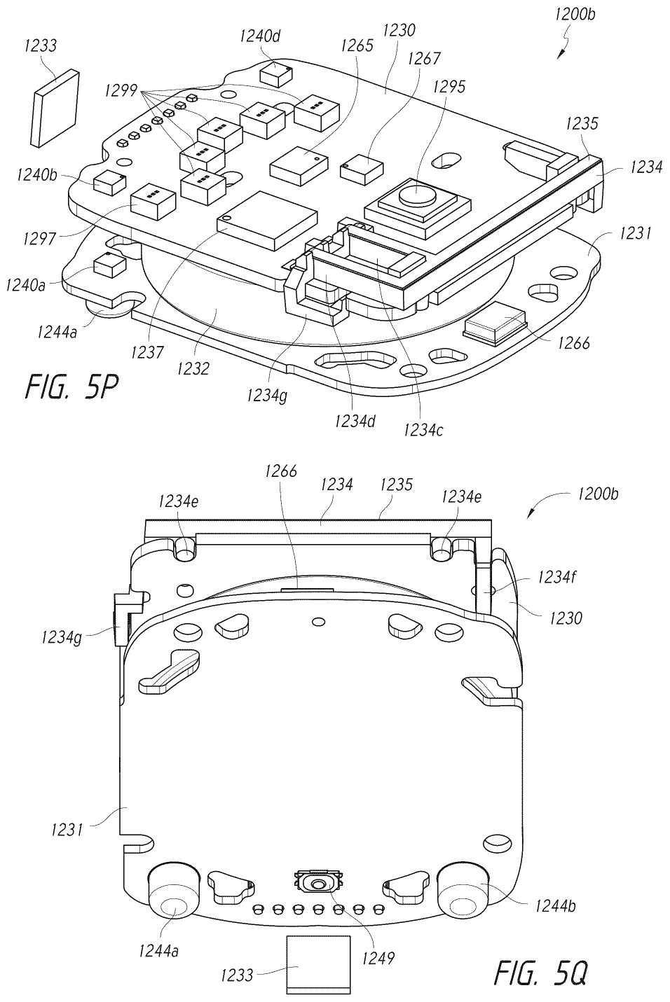

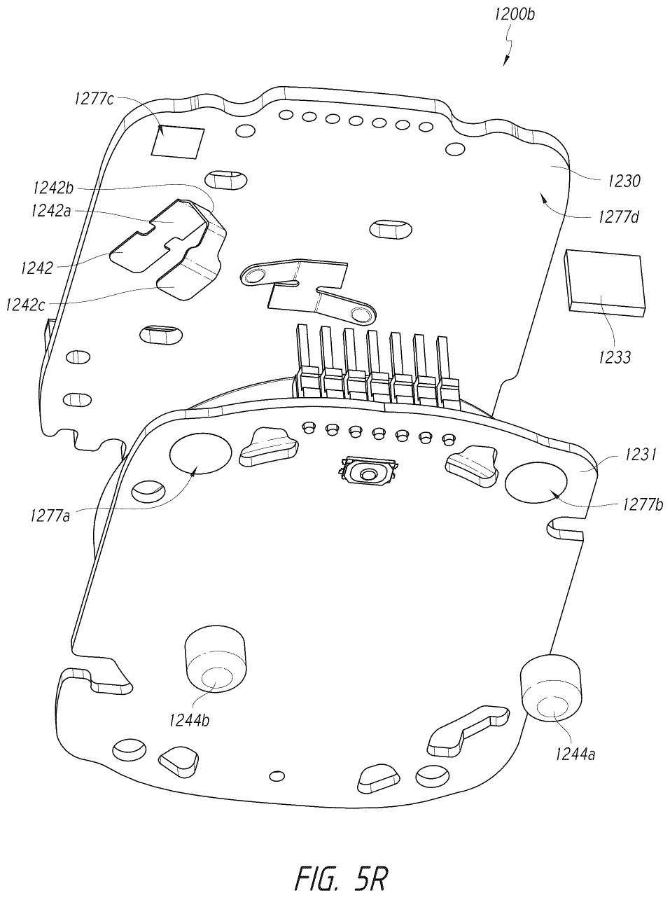

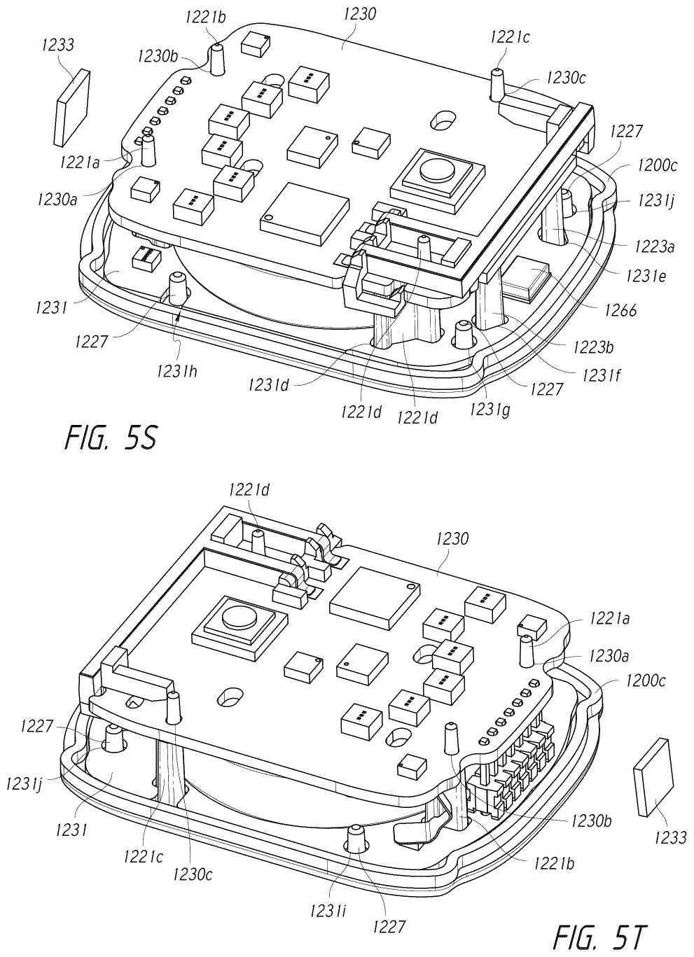

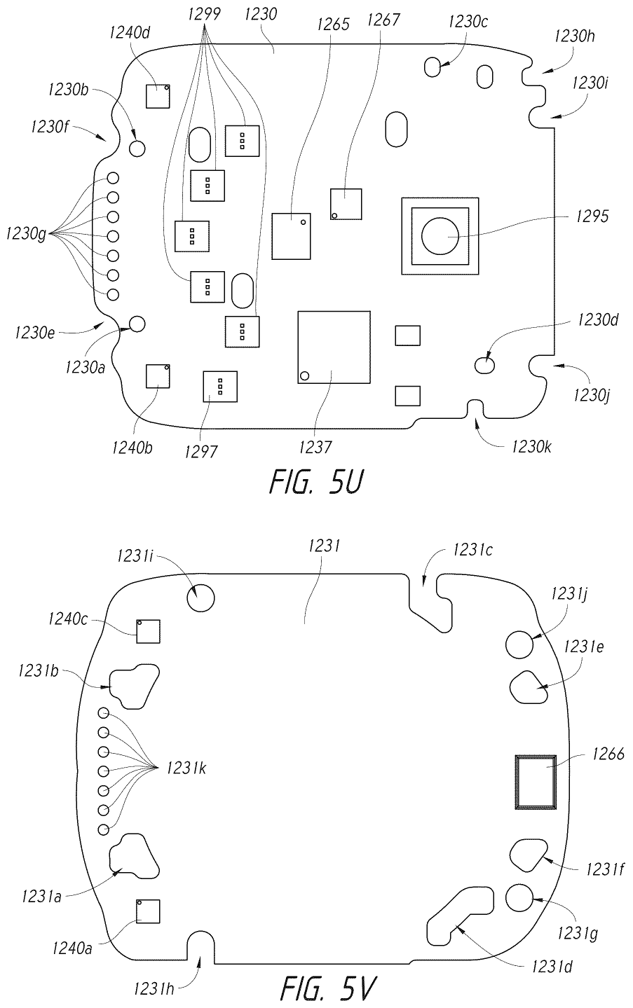

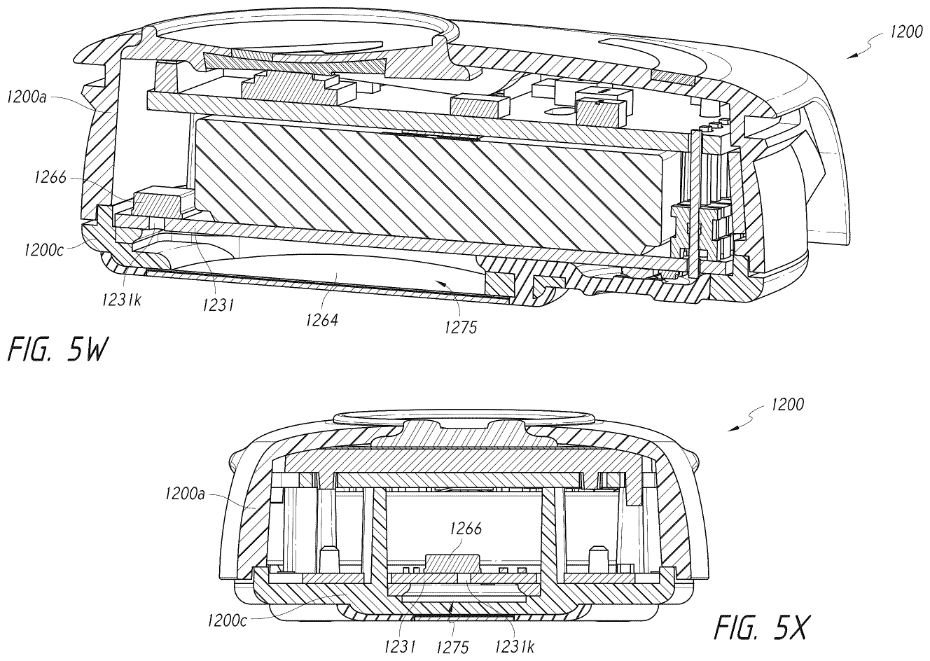

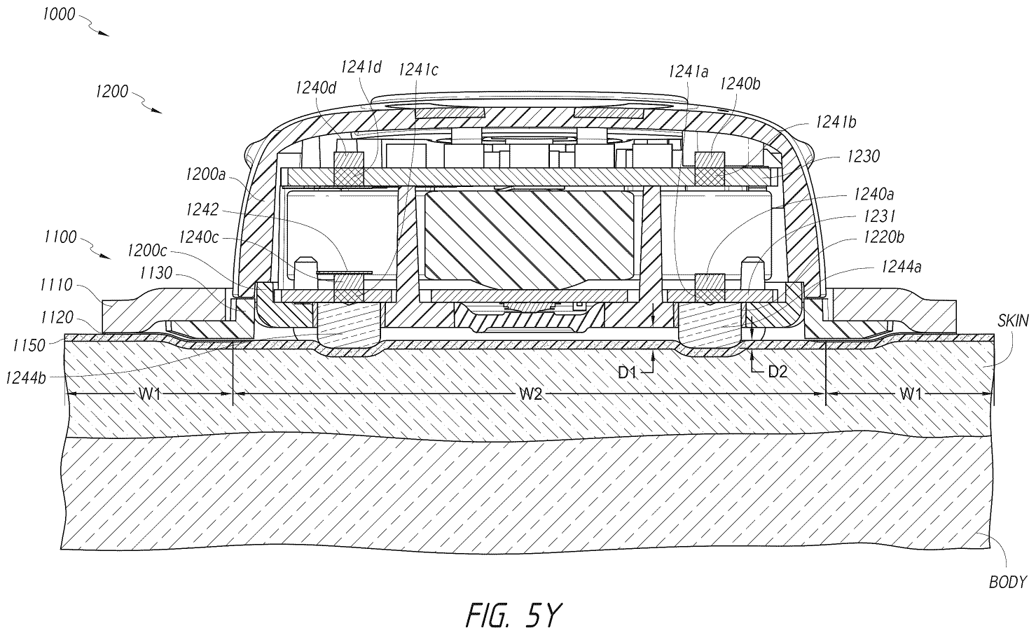

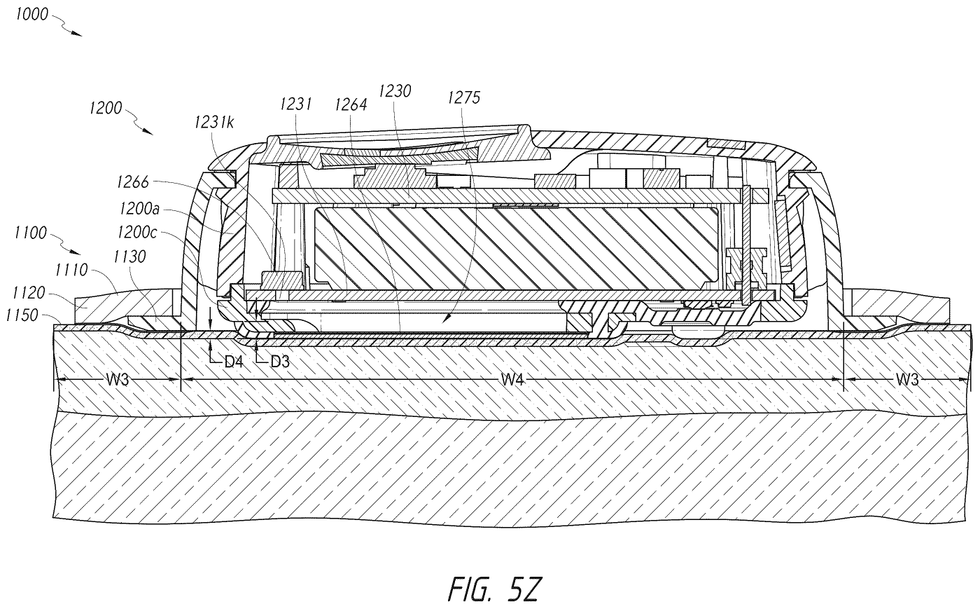

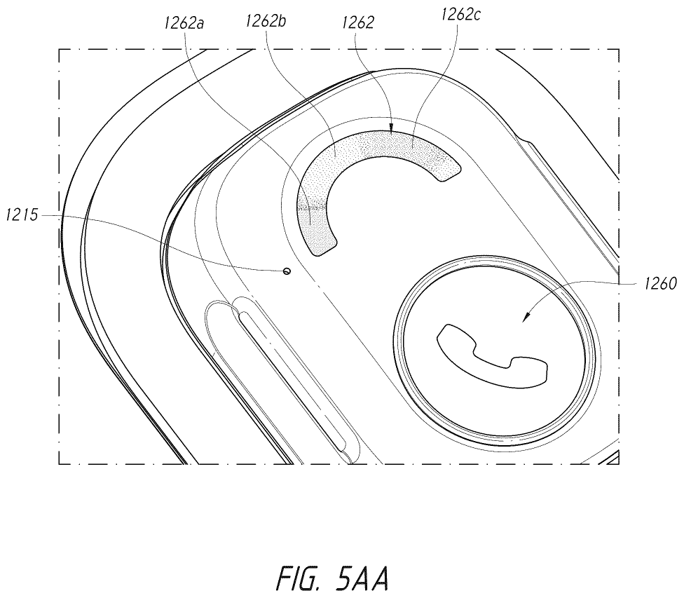

In some implementations, wearable device 1000 includes a display 1007. In some implementations, display 1007 displays physiological parameters and/or other information (such as motion and/or orientation related information of a user) which is determined by wearable device 1000. In some implementations, display 1007 only displays information relating to the user's orientation and/or only di splays graphical representations of the user's orientation, and does not display any information relating to physiological parameters of the user (for example, does not display information relating to pulse rate, oxygen saturation, temperature, cardiac activity, and/or lung activity of the user). Display 1262, illustrated in at least FIGS. 5A-5C and discussed further herein, can be an implementation of display 1007. Display 1262 is also shown in FIG. 5AA. Display 1262 can graphically illustrate (for example, via color) an amount of time the user has spent in various orientations, such as right side, left side, supine, among other positions. In some implementations, display 1262 can graphically illustrate an amount of health risk associated with an orientation of the user. Display 1262 can be similar or identical to any of the displays shown and/or described in U.S. Pat. No. 11,406,286, U.S. Pat Pub. No. US2023/0045000, and/or U.S. Pat App. No. 63/253,324 incorporated by reference herein. Furthermore, a determination of an amount of health risk associated with an orientation of the user (e.g., for display, such as on display 1007, 1262) can be similar or identical as described in U.S. Pat. No. 11,406,286, U.S. Pat Pub. No. US2023/0045000, and/or U.S. Pat App. No. 63/253,324 incorporated by reference herein.

In some implementations, wearable device 1000 (for example, via processor 1001): receives one or more signals generated by motion sensor 1010 responsive to linear acceleration of the user and/or receives one or more signals generated by motion sensor 1010 responsive to angular velocity of the user; determines orientation of the user relative to a surface (for example, a bed) over time based on said received one or more signals; and changes appearance of display 1007, 1262 based on the determined orientation over time. This can, among other things, provide the user and/or a care provider with information for assessing a risk of pressure ulcer formation (and when/how a user should be repositioned), a common occurrence among users in care settings.

In some implementations, wearable device 1000 (for example, via processor 1001) associates a timer with each of a plurality of orientations of the user (for example, relative to a bed). Such plurality of orientations can include, for example, a right side orientation, a left side orientation, a supine orientation, a prone orientation, a plurality of orientations between the right and left side orientations, and/or a plurality of orientations between the supine and prone orientations. In some implementations, wearable device 1000 (for example, via processor 1001) changes a value of a timer associated with a respective orientation in a first manner when the user is in the respective orientation and changes the value of the timer associated with the respective orientation in second manner when the user is not in the respective orientation. For example, in some implementations, wearable device 1000 (for example, via processor 1001) increases a value of a timer associated with a respective orientation when the user is in the respective orientation and decreases the value of the timer associated with the respective orientation when the user is not in the respective orientation. In such manner, wearable device 1000 can keep track of accumulated and de-accumulated time of the user in and out of each of a plurality of orientations. Such information can advantageously be utilized by the user and/or a care provider to quickly and easily assess the user's risk of pressure ulcer formation (the risk of which increases the more time the user is in a given orientation).

Wearable device 1000 (for example, via processor 1001) can change an appearance of display 1007, 1262 based on the values of such timers. In some implementations, wearable device 1000 (for example, via processor 1001) changes an appearance of (for example, color of) each of a plurality of display elements (which can also be referred to herein as “portions”) of display 1007, 1262 (each of such plurality of display elements of the display being associated with one of a plurality of orientations) based on the value of the timer associated with each one of said plurality of orientations. Such display elements can include display elements 1262a, 1262b, 1262c as shown in FIG. 5AA.

Wearable device 1000 (for example, via processor 1001) can change an appearance of display 1007, 1262 based on an amount of health risk associated with the orientation of the user. In some implementations, wearable device 1000 (for example, via processor 1001) changes an appearance of (for example, color of) each of a plurality of display elements of display 1007, 1262 (each of such plurality of display elements of display being associated with one of a plurality of orientations) based on the amount of health risk associated with each one of said plurality of orientations. Such display elements can include display elements 1262a, 1262b, 1262c as shown in FIG. 5AA.

In some implementations, wearable device 1000 (for example, via processor 1001): causes an appearance of each respective one of the plurality of display elements to have a first color when the value of the timer associated with such respective one is greater than or equal to a threshold; and causes the appearance of the respective one of the plurality of display elements to have a second color when the value of the timer associated with such respective one is below such threshold. In some implementations, wearable device 1000 (for example, via processor 1001): causes an appearance of each respective one of the plurality of display elements to have a first color when the health risk associated with such respective one is greater than or equal to a threshold; and causes the appearance of the respective one of the plurality of display elements to have a second color when the value of the health risk associated with such respective one is below such threshold. Such first and second colors can be different. In some implementations, a first color is red and a second color is green. In some implementations, more than one color is used to indicate a grade of health risk. For example, green can be used to indicate no health risk, yellow can be used to indicate moderate health risk, and red can be used to indicate health risk.

In some implementations, display 1007, 1262 is defined by a border having at least a first edge and a second edge, and each of said plurality of display elements/portions of the display 1007, 1262 is a line or a region extending between the first and second edges of the border. In some implementations, display 1007, 1262 can have an arch shape as shown in at least FIGS. 5A-5C and 5AA. Wearable device 1000 (for example, via processor 1001) can be configured to change the appearance of display 1007, 1262 in a similar or identical manner as that described in U.S. Pat. No. 11,406,286, U.S. Pat Pub. No. US2023/0045000, and/or U.S. Pat App. No. 63/253,324 incorporated by reference herein

The health risk (which can also be referred to herein as “risk”) associated with an orientation of the user can be a measure of time. For example, the health risk associated with an orientation of the user can correspond to an amount of time a user is in a certain position/orientation as described herein (e.g., right side, left side, prone/supine). Such an amount of time a user is in a certain position/orientation can be consecutive or non-consecutive. In some implementations, the health risk associated with an orientation of the user is at least partially dependent upon the amount of time a patient is in a particular position/orientation. In some implementations, the risk associated with an orientation of the user is associated with a combination of factors. Such factors can include a physiological parameter of the user. In some implementations, such factors can include a physiological parameter of the user determined by the wearable device 1000 (e.g., any of those described herein such as temperature, cardiac activity/function, lung activity/function, and/or bodily sounds). Additionally or alternatively, such factors can include any one or more parameters of the user such as weight, age, blood pressure, blood sugar, diabetic status, and/or medical history and/or any information contained within the user's electronic medical record.

With reference to FIG. 2, wearable device 1000 can include one or more user inputs 1008 that can allow a user (or a care provider) to interact with wearable device 1000. A user input 1008 can be utilized to transition wearable device 1000 from a non-operational mode to an operational mode (and vice versa) for example, or carry out other actions. With reference to at least FIG. 5D, wearable device 1000 (e.g., hub 1200 of the wearable device 1000 as described herein) can include a button 1222, which can be an implementation of user input 1008. Another user input 1008 can be configured to contact a care provider. With reference to at least FIG. 5C, wearable device 1000 (e.g., hub 1200) can include a button 1260, which can be such another implementation of user input 1008.

With continued reference to FIG. 2, in some implementations, wearable device 1000 includes one or more microphones (which can also be referred to herein as an “audio transducer”) 1011. Microphone(s) 1011 can be utilized to receive audio from a user to enable such audio to be transmitted to a separate device (such as one associated with a care provider). This can facilitate audio communication between the user wearing the wearable device 1000 and other parties. Microphone(s) 1011 can additionally or alternatively be utilized by wearable device 1000 to perform the function of a digital stethoscope. As described further below, wearable device 1000 can include a diaphragm 1264 (see at least FIGS. 5D, 5E, 5W, 5X, 5Z) that can vibrate responsive to cardiac activity, lung activity, and/or other body sounds of the user. Such vibration of the diaphragm 1264 can generate sound waves within at least a portion of an interior of the wearable device 1000 (for example, a portion of an interior defined by a housing of hub 1200). Microphone(s) 1011 can detect such vibrations (and/or sound waves generated from such vibrations) and generate signal(s) based on the detected vibration/sound waves. Such signal(s) generated by microphone 1011 can be received and/or processed by processor 1001 to, for example, determine at least one of cardiac function, lung function, or other bodily functions of the user. In some implementations, wearable device 1000 transmits (for example, wirelessly) processed and/or unprocessed signal(s) generated by microphone(s) 1011 and/or information indicative of cardiac activity, lung activity, and/or other bodily activity of the user that is derived based on such signal(s). Furthermore, the wearable device 1000 can be configured to separate such signal(s) based on the type of activity (e.g., cardiac, lung, or other body activity) for independent listening by a care provider. In some implementations, the wearable device 1000 can include a microphone to perform the function of a digital stethoscope and another microphone to vibrate responsive to vibration of a housing of the wearable device 1000 and/or ambient noise external to the wearable device 1000. In such implementations, the wearable device 1000 can process signals from each of such microphones and determine, via the one or more processors, corrected signals indicative of cardiac activity and/or function, lung activity and/or function, and/or of other body sounds. Furthermore, in such implementations each microphone can be positioned within the wearable device 1000 to perform said function (e.g., the microphone performing the function of a digital stethoscope can be positioned closer to the user's body when the device is attached thereto than the microphone for determining ambient sounds). In some implementations, signals from microphone 1011 can be streamed to earbuds, headphones, or a sound system used by a care provider.

In some implementations, wearable device 1000 includes a status indicator 1009 configured to indicate a status of wearable device 1000, such as a life of battery 1004 of wearable device 1000, a mode in which wearable device 1000 is operating, an error condition, among other things. Status indicator 1009 can be implemented as one or more emitters configured to emit light, such as emitter 1297 illustrated in at least FIGS. 5N-5P.

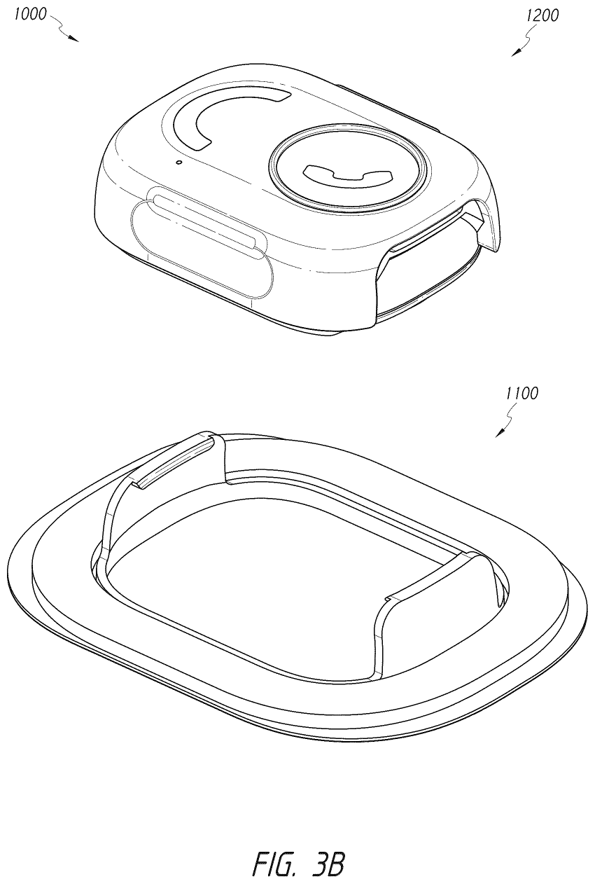

Wearable device 1000 can include a first portion that can secure (for example, removably secure) wearable device 1000 to a user and a second portion that can, for example, include various components of wearable device 1000 (such as any of the electronic components discussed herein). In some implementations, such first and second portions of wearable device 1000 can be removable from each other. In some implementations, such first portion includes one or more substrates configured to adhere (for example, removably adhere) to skin. In some implementations, such first portion does not include any electronic components, for example, where any and all electronic components of the wearable device 1000 (such as any of those described herein) are contained in the second portion. Such first and second portions of the wearable device 1000 can be configured to mechanical removably secure to one another. In some implementations, such first and second portions are configured to be difficult to detach from one another if the first portion is secured to the user. In some implementations, the intended service lives of the first and second portions are different. For example, the intended service life of the first portion can be less than the intended service life of the second portion, such as where the first portion includes one or more substrates that secure to the user's skin and where the second portion includes electronic components of the wearable device 1000. In such implementations, the first portion can be disposed of and replaced and the second portion can be secured with a new first portion. This is advantageous where the substrates lose integrity and/or become degraded after an amount of time that is less than a battery life of the second portion of the wearable device 1000. Such first and second portions can be the dock 1100 and hub 1200 (respectively) illustrated in the exploded view of FIGS. 3A-3B and discussed further herein.

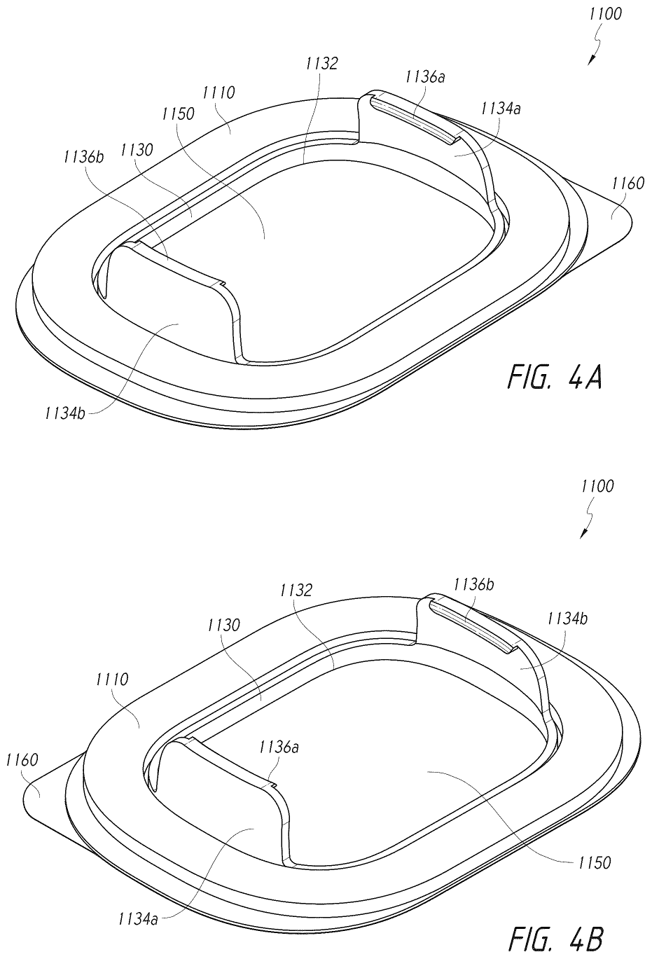

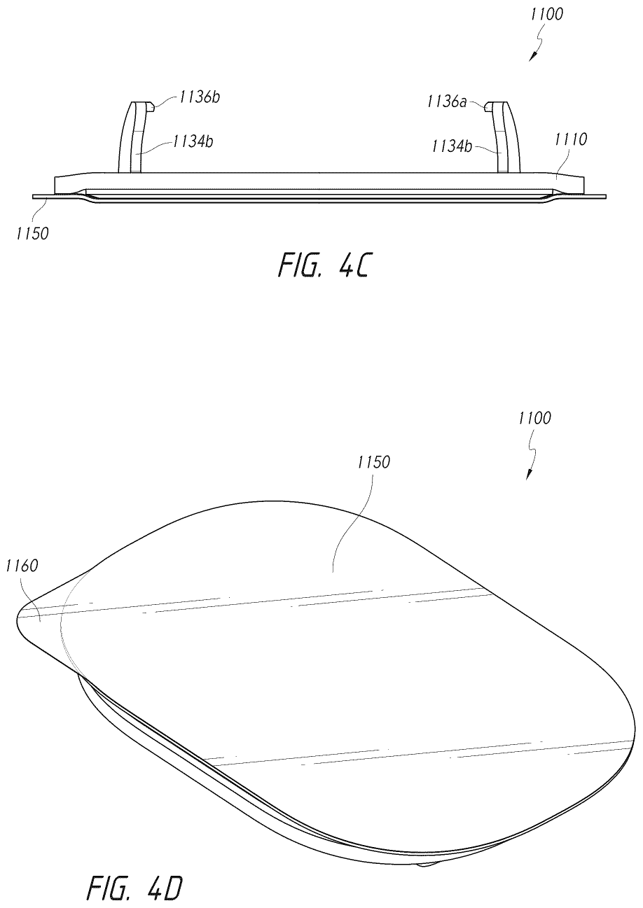

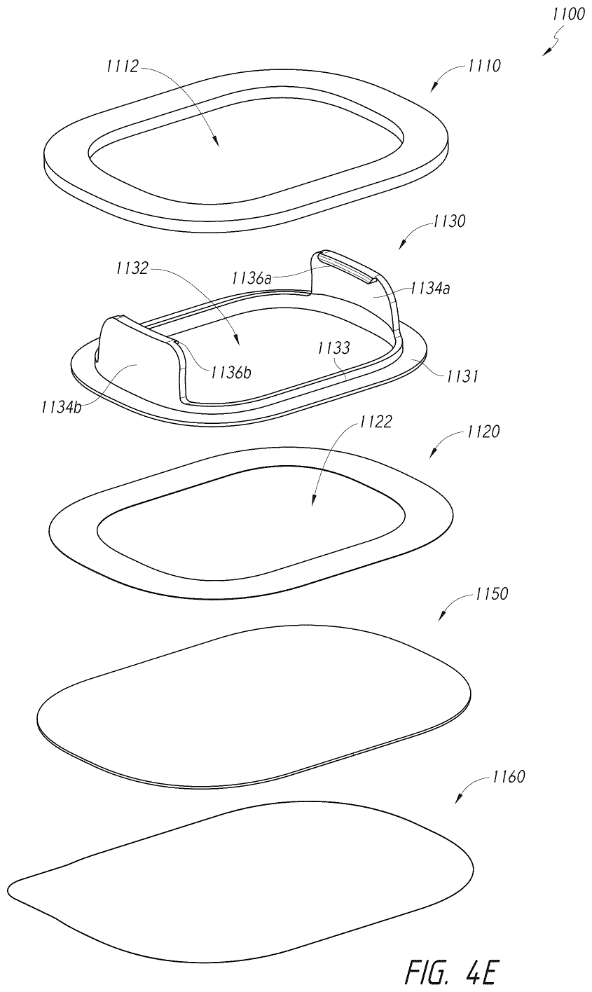

FIGS. 4A-4B illustrate top perspective views of dock 1100. FIG. 4C illustrates a first side view of dock 1100, which can be a mirror image of an opposite second side view of dock 1100. FIG. 4D illustrates a bottom perspective view of dock 1100. FIG. 4E illustrates an exploded view of dock 1100. Dock 1100 can be similar or identical to dock 200 in some or many respects. Dock 1110 can include a frame 1130 and one or more substrates coupled to frame 1130, such as any of substrates 1110, 1120, 1150, 1160. With reference to FIG. 4E, frame 1130 can include a rim 1131 and an opening 1132. Rim 1131 can define a perimeter of the frame 1130. Rim 1131 can have a rounded shape. Frame 1130 can be configured to removably secure to hub 1200, for example, to a housing of hub 1200. Frame 1130 can include one or more arms which are configured to engage with portions of the housing of the hub 1200. For example, frame 1130 can include arms 1134a, 1134b that can extend outward from rim 1131. Arm 1134a can be positioned at a first end of frame 1130 and arm 1134b can be positioned at a second end of frame 1130 that is opposite of such first end of frame 1130. Arms 1134a, 1134b can extend along a portion of rim 1131, for example, less than an entire perimeter of rim 1131. Arms 1134a, 1134b can extend generally perpendicular to rim 1131 (e.g., a plane defined by rim 1131) and/or can extend generally perpendicular to opening 1132 (e.g., a plane defined by opening 1132). In some implementations, frame 1130 comprises a wall 1133 that extends outward from (e.g., generally perpendicular to) rim 1131. Wall 1133 can have a height that is less than arms 1134a, 1134b, as shown in FIG. 4E. In some implementations, arms 1134a, 1134b are curved along lengths (which may also be referred to as widths) thereof. For example, arms 1134a, 1134b can be curved to correspond with a curved shape of rim 1131 at first and second ends of frame 1130.

Arms 1134a, 1134b can be configured to engage with portions of a housing of hub 1200 to facilitate securement (e.g., removable securement) of the dock 1100 and hub 1200. As shown, arms 1134a, 1134b can include protrusions 1136a, 1136b. Arm 1134a can include a first surface (which may be referred to as an “inward surface”) and a second surface (which may be referred to as an “outward surface”) opposite the first surface and arm 1134b can include a first surface (which may be referred to as an “inward surface”) and a second surface (which may be referred to as an “outward surface”) opposite the first surface of arm 1134b. The first surfaces of arms 1134a, 1134b can face at least partially toward each other (for example, can face in an inward direction of frame 1130) and the second surfaces of the arms 1134a, 1134b can face away from one other. Protrusions 1136a, 1136b can extend outward from such respective inward surfaces of arms 1134a, 1134b and at least partially in a direction toward one another and/or toward an interior of frame 1130. Protrusion 1136a can extend along a portion of a length of arm 1134a and protrusion 1136b can extend along a portion of a length of arm 1136a. While the figures illustrate protrusions 1136a, 1136b having a continuous length, in some variants, one or both of arms 1134a, 1134b include a plurality of protrusions separated from one another, for example, in a location such as that shown with respect to protrusions 1136a, 1136b. Protrusions 1136a, 1136b can engage recesses 1207a, 1207b of hub 1200 as discussed in more detail herein, which can facilitate securement of the hub 1200 and the dock 1100. With reference to FIG. 4C, protrusions 1136a, 1136b can have a beveled or chamfered edge on free ends thereof, which can facilitate movement along a portion of ends 1202, 1204 of the hub 1200 and positioning within recesses 1207a, 1207b as explained in more detail herein. Although arms 1134a, 1134b are shown as having protrusions 1136a, 1136b and hub 1200 is shown as having recesses 1207a, 1207b, in some variants, arms 1134a, 1134b have recesses instead of protrusions 1136a, 136b and hub 1200 has protrusions instead of recessed 1207a, 1207b.

As also described further herein, in some implementations, arms 1134a, 1134b can be configured to move when forces are applied to dock 1100, which can facilitate removal of protrusions 1136a, 1136b from recesses 1207a, 1207b of hub 1200 (see FIGS. 5A-5B). For example, in some implementations, application of opposing forces on opposite sides of the dock 1100 (which extend between ends of dock 1100 where arms 1134a, 1134b are located) can cause arms 1134a, 1134b to move from a first position (as shown in FIG. 4C) to a second position where arms 1134a, 1134b are positioned farther from each other than when in the first position. In such second position, arms 1134a, 1134b can be flexed outward from one another (for example, to the “right” and “left” given the view shown in FIG. 4C). Such configuration can move the protrusions 1136a, 1136b out of recesses 1207a, 1207b, thereby allowing hub 1200 to be removed from dock 1100. In some implementations, dock 1100 does not include a clip or other structure that can be actuated by a user to disengage the protrusions 1136a, 1136b from recesses 1207a, 1207b. For example, in some implementations, in order for the hub 1200 and dock 1100 to be removed from each other, a portion or portions of the dock 1100 (for example, the frame 1130) must be deformed (for example, outward flexing of arms 1134a,1134b). In some cases, such configurations make it difficult to remove hub 1200 and dock 1100 from one another when dock 1100 is secured to the user's skin.

Such configurations can be advantageous to prevent users from separating the hub 1200 and dock 1100 from one another and interfering with operation of the wearable device 1000. As discussed further herein, the hub 1200 can include a button that allows the hub 1200 to be transitioned from a non-operational mode to an operational mode, and in some implementations, such button is inaccessible when the hub 1200 and dock 1100 are coupled together. Such configurations can also inhibit users from intentionally or unintentionally interfering with operation of the wearable device 1000 (for example, shutting it off). For example, in some implementations, in order for the wearable device 1000 to be turned off, dock 1100 and hub 1200 must be removed (while coupled together) from the user's skin, arms 1134a, 1134b must be flexed outward (thereby removing protrusions 1136a, 1136b from recesses 1207a, 1207b of hub 1200), and hub 1200 must be decoupled from dock 1100. In such configurations, it may be difficult for a user to carry out such actions when the wearable device 1000 is secured to the user's own skin, but such actions may be performed by a care provider, which may be desirable in certain situations.

As mentioned above, FIG. 4E illustrates an exploded view of dock 1100. Dock 1100 can include one or more substrates that can secure and/or secure to other portions of dock 1100 and/or that can allow the dock 1100 to secure to a user (e.g., skin of the user). For example, with reference to FIG. 4E, dock 1100 can include one or more of substrates 1110, 1120, 1150, and/or 1160.

Substrate 1110 can be configured to surround a portion of frame 1130. For example, substrate 1110 can include an opening 1112. Opening 1112 can be sized and/or shaped to surround arms 1134a, 1134b and wall 1133. Substrate 1110 can be made of foam material such as white polyethylene, polyurethane, or reticulated polyurethane foams, to name a few. Substrate 1110 can be made of medical-grade foam material. Substrate 1110 can have a perimeter that is greater than a perimeter of rim 1131 of frame 1130 in some implementations. Substrate 1110 can have an adhesive on an underside thereof, which can allow substrate 1110 to secure to at least frame 1130 in some implementations. Substrate 1120 can be positioned between rim 1131 and a substrate 1150 (described further herein). Substrate 1120 can include an opening 1122 as shown. In some implementations, openings 1112 and 1122 are substantially identical or identical. Substrate 1120 can comprise polyethylene and/or adhesive on one or both sides thereof, which can help secure substrate 1150 to frame 1130 (e.g., rim 1131 of frame 1130). Substrate 1120 can be sandwiched between rim 1131 and substrate 1150. In some implementations, an additional substrate 1120 can be placed between substrate 1110 and frame 1130 to aid in securing substrate 1110 to at least frame 1130. In some implementations, substrate 1120 can aid in securing substrate 1110 and substrate 1150 to one another beyond a perimeter of the rim 1131 of the frame 1130.

Substrate 1150 can contact and/or secure to skin of a user when the wearable device 1000 is in use. Substrate 1150 can be a bottommost portion of the wearable device 1000 when the wearable device 1000 is in use (for example, after the release liner 1160 is removed). Substrate 1150 can be or include a material configured to secure to skin of a user. Substrate 1150 can comprise a material configured to allow for removable securement of the wearable device 1000 to the user's skin. For example, the substrate 1150 can be coated with a high tack, medical-grade adhesive, which when in contact with the user's skin, is suitable for long-term monitoring, such as, for example two days or longer, such as 3 days, 4 days, 5 days, 6 days, 7 days, 8 days, 9 days, or 10 days or longer. Additionally or alternatively, the substrate 1150 can be or include a soft, comfortable, and/or breathable material. For example, substrate 1150 can be or include fabric, such as a silicone spun-lace fabric. The substrate 1150 can include an adhesive material or layer (such as adhesive tape). Such configuration can allow the wearable device 1000 to comfortably secure to the user's skin. Substrate 1150 can provide thermal insulation and/or provide thermal conductivity. For example, when the wearable device 1000 is positioned on and/or secured to (e.g., adhered to) a user's skin surface, substrate 1150 can act to insulate the skin surface at, around, and/or proximate to a point or region where temperature is measured and/or where thermal energy is transmitted from the skin surface of the user to or near one or more temperature sensors of the wearable device 1000 (e.g., via thermally conductive probes 1244a, 1244b described herein). For example, when the wearable device 1000 is positioned on and/or secured to (e.g., adhered to) a user's skin surface, substrate 1150 can insulate the skin surface and can transmit thermal energy to thermally conductive probes 1244a, 1244b, which in turn, can transmit thermal energy to and/or toward temperature sensors 1240a, 1240c as described further below. In some implementations, substrate 1150 can provide electrical insulation and/or provide electrical conductivity. In some implementations, substrate 1150 can provide acoustic insulation and/or provide acoustic conductivity. In some implementations, at least a portion of the substrate 1150 can be modified and/or removed to enhance any one of a thermal conductivity, an electrical conductivity, and an acoustic conductivity between the user and one or more sensors of the wearable device 1000.

Dock 1100 can include a substrate that is a release liner 1160. The release liner 1160 can secure to one or more of the above-described substrates (such as substrate 1150) and can be removed prior to securement of the wearable device 1000 to a user. For example, release liner 1160 can be removed from the substrate 1150 prior to placement and/or securement of the wearable device 1000 on the user's skin.

FIGS. 5A-5B illustrate top perspective views of hub 1200, FIG. 5C illustrates a top view of hub 1200, FIG. 5D illustrates a bottom view of hub 1200, FIG. 5E illustrates a bottom perspective view of hub 1200, and FIG. 5E illustrates an end view of hub 1200. Hub 1200 can have a first end 1202, a second end 1204 opposite the first end 1202, a first side 1206, and a second side 1208 opposite the first side 1206.

Hub 1200 can be configured to be removably secured to dock 1100, for example, via interaction between recesses 1207a, 1207b and protrusions 1136a, 1136b of arms 1134a, 1134b. Hub 1200 can include a housing which can itself include shells 1200a, 1200c (see FIGS. 5G-5H) which can be secured (for example, permanently secured together) to enclose electronic components of the wearable device 1000. With reference to FIG. 5A, a portion 1202a of end 1202 of hub 1200 (which can be defined by portions of shells 1200a, 1200c of a housing of hub 1200) can be sized and/or shaped to receive and/or conform to a shape of arm 1134a. Similarly, with reference to FIG. 5B, a portion 1204a of end 1204 of hub 1200 (which can be defined by portions of shells 1200a, 1200c of a housing of hub 1200) can be sized and/or shaped to receive and/or conform to a shape of arm 1134b. Such portions 1202a, 1204a can be recessed from an outer surface of housing of hub 1200 (for example, formed by shells 1200a, 1200c).

Recessed portions 1202a, 1204a can include structure that facilitates engagement and/or securement with protrusions 1136a, 1136b of arms 1134a, 1134b of frame 1130 of dock 1100. For example, portion 1202a of end 1202 can include a recessed portion 1203a, a recess 1207a (which may also be referred to as a “groove”) and a wall 1205a (which may also be referred to as a “non-recessed portion”) that at least partially separates recessed portion 1203a and recess 1207a. Similarly, portion 1204a of end 1204 can include a recessed portion 1203b, a recess 1207b (which may also be referred to as a “groove”) and a wall 1205b (which may also be referred to as a “non-recessed portion”) that at least partially separates recessed portion 1203b and recess 1207b. In some implementations, hub 1200 can be secured to dock 1100 by inserting hub 1200 between arms 1134a, 1134b of dock 1100 from above (see, for example, FIGS. 3A-3B). During such insertion, protrusions 1136a, 1136b can contact and/or slide along recessed portions 1203a, 1203b, slide over walls 1205a, 1205b, and move into recesses 1207a, 1207b. In some implementations, protrusions 1136a, 1136b are configured for snap-fit engagement with recesses 1207a, 1207b (which can be facilitated by recessed portions 1203a, 1203b and/or walls 1205a, 1205b). In some implementations, protrusions 1136a, 1136b have a beveled or chamfered end which can help protrusions 1136a, 1136b slide over walls 1205a, 1205b and into recesses 1207a, 1207b. Hub 1200 and dock 1100 can be decoupled from one another as described above herein. In some implementations, hub 1200 includes features that can facilitate gripping and/or handling of hub 1200, for example, before, during, and/or after removal of hub 1200 and dock 1100 from one another. For example, hub 1200 can include recessed portions 1209a, 1209b and/or protrusions 1211a, 1211b extending along a portion of lengths of sides 1206, 1208.

In some implementations, hub 1200 includes an opening 1215 configured to allow light from an emitter 1297 (e.g., and LED), which can be an implementation of status indicator 1009, housed within the hub 1200 to exit the hub 1200 and illuminate nearby areas. This can be utilized to indicate a status of the wearable device 1000. Opening 1215 can be located in shell 1200a, which can form a housing when secured to shell 1200c. Opening 1215 can be substantially aligned with emitter 1297 (see at least FIGS. 5N-5P) to allow light emitted from emitter 1297 to pass through the housing (for example, through shell 1200a).

FIGS. 5E-5F illustrate probes 1244a, 1244b extending through openings 1224a, 1224b in housing of hub 1200 (e.g., through shell 1200c). Probes 1244a, 1244b are described in more detail below. FIGS. 5E-5F also illustrate button 1222 which is described above, and diaphragm 1264, which is described further below.

FIGS. 5G-5H illustrate exploded perspective views of hub 1200. Hub 1200 can include a housing formed by shell 1200a (which may be referred to herein as “top shell” or “second shell”) and shell 1200c (which may be referred to herein as “bottom shell” or “first shell”). Such housing can enclose electronic components of wearable device 1000. Shells 1200a, 1200c can be permanently secured together. In some implementations, shells 1200a, 1200c are secured together so as to prevent water ingress into an interior of a housing formed by shells 1200a, 1200c, which can in turn protect electronic components contained therewithin. In some implementations, joining edges (for example, a bottom edge of shell 1200a and a top edge of shell 1200c) can be ultrasonically welded together to prevent water ingress.

In some implementations, dock 1100 does not include any electronic components and all electronic components of wearable device 1000 are contained in hub 1200. As shown in FIGS. 5G-5H, wearable device 1000 (for example, hub 1200) can include an electronics assembly, represented by numeral “1200b” for purposes of convenience. FIGS. 5I-5J illustrate bottom perspective views of shell 1200a and FIGS. 5K-5M illustrate views of shell 1200c. FIGS. 5N-5R illustrate views of various electronic and/or structural components that can be enclosed in a housing of wearable device 1000 (for example, of hub 1200) and can form electronics assembly 1200b. Use of the phrase “electronics assembly” and use of numeral “1200b” in the present disclosure is not intended to be limiting, but rather, is merely intended as a convenient method to refer to one or more components of wearable device 1000 which can be enclosed by shells 1200a, 1200c. The use of such phrase and such numeral is not intended to convey that the inclusion of any elements or features described with reference to electronics assembly 1200b necessarily requires inclusion of any or all other elements or features described with reference to electronics assembly 1200b.

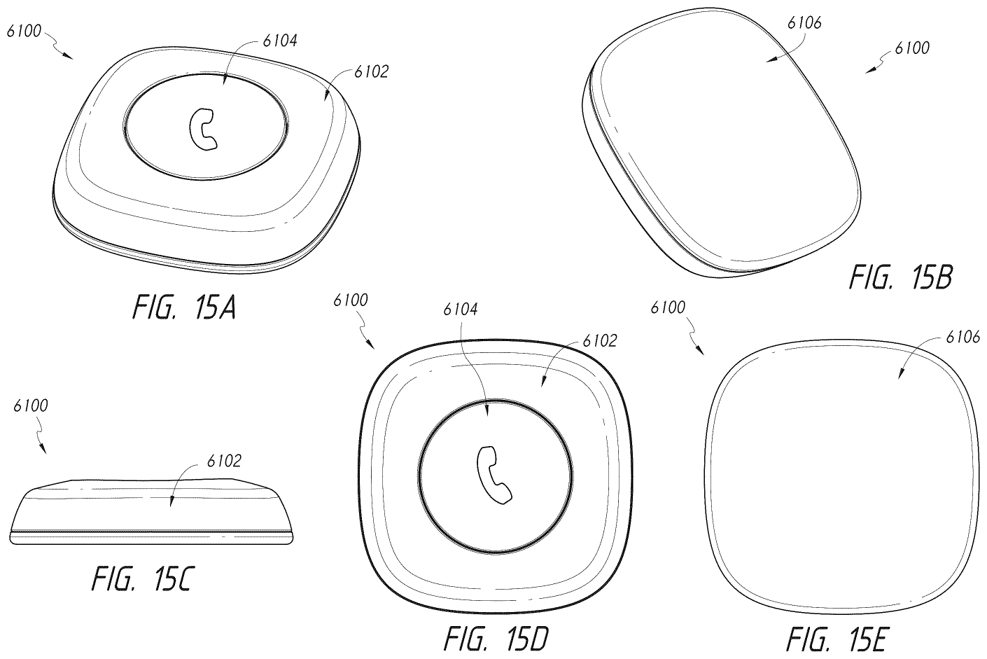

As mentioned above, FIGS. 5I-5J illustrate bottom perspective views of shell 1200a. Shell 1200a can include various structure that can engage with portions of electronics assembly 1200b and/or act to operably position portions of electronics assembly 1200b. For example, shell 1200a can include structure that engages and/or operably positions any or all of circuit boards 1230, 1231 (see FIGS. 5N-5Q). For example, shell 1200a can include walls 1210e that can engage notches 1230i, 1230j in circuit board 1230 (see FIG. 15U). In some implementations, shell 1200a includes a pocket 1210f configured to receive an NFC transponder 1233 (see FIG. 5I and FIGS. 5N-5Q). As also shown in FIGS. 5I-5J, shell 1200a can include a cavity defined by an enclosure 1210d which can be positioned around emitter 1297. Opening 1215 can extend through a portion of shell 1200a into such cavity defined by enclosure 1210d, as shown.

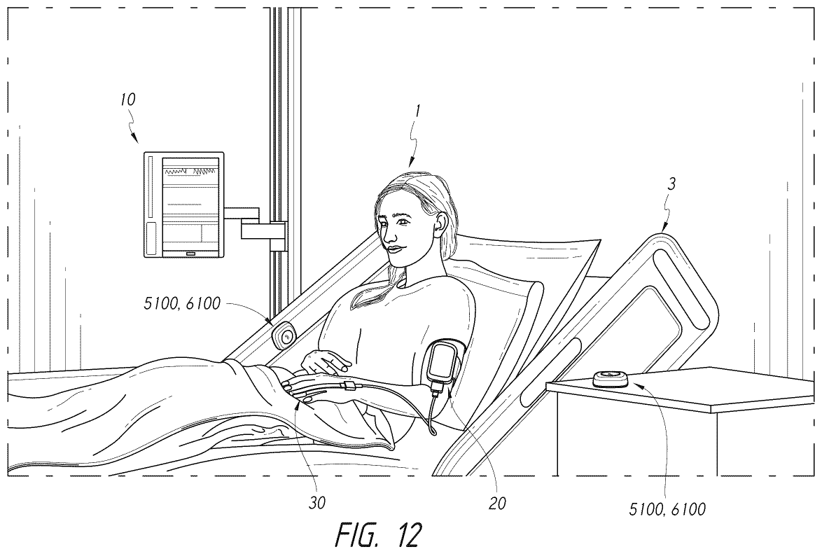

As shown in FIGS. 5A-5C and as discussed elsewhere herein, wearable device 1000 can include a user input 1260 (which also may be referred to herein as a “button”, a “call button”, a “care provider call button”, a “nurse call button”, or a “caregiver call button”). Call button 1260 can include various types of electrical and mechanical structure. With reference to FIGS. 5N-5P, call button 1260 can include a switch 1295, which can be mounted to circuit board 1230. Call button 1260 can include various mechanical structure configured to interact with switch 1295. For example, with reference to FIGS. 5A-5C and 5I-5J, call button 1260 can include a pad 1212 coupled to a plate 1210c, and the plate 1210c can be mounted to a portion of shell 1200a via one or more arms 1210b (for example, two arms 1210b). Pad 1212 and plate 1210c can be movably mounted to a portion of shell 1200a via arms 1210b, and arms 1210b can be flexible and resilient. Pad 1212 and plate 1210c can be configured to move from a first position (which can be referred to as a “neutral” position) to a second position (which can be an “actuated” position). Arms 1210b can bias the pad 1212 and plate 1210c towards such first position. When pad 1212 and/or plate 1210c are moved to such second position (e.g., by a press in on the pad 1212 of call button 1260), pad 1212 and/or plate 1210c can engage switch 1295. In some implementations, pad 1212 is made of a different material than plate 1210c, arms 1220b, and/or a remainder of shell 1200a. For example, pad 1212 can be made of a softer and/or more flexible material than plate 1210c, arms 1210b, and/or a remainder of shell 1200a. A top portion of pad 1212 is shown/indicated in FIG. 5H and a bottom portion of pad 1212 is shown/indicated in FIGS. 5I-5J. In some implementations, call button 1260 is defined by switch 1295 and one or more of pad 1212, plate 1210c, and/or arm(s) 1210b. In some embodiments, button 1260 can be at least partially recessed relative to an external surface of the wearable device 1000 (e.g., relative to shell 1200a). Such a configuration can advantageously help prevent a user from accidentally actuating the button 1260. Furthermore, in some implementations button 1260 can have a concave and/or dished configuration. Such a configuration can advantageously help a user find the button 1260 for actuating (e.g., in a time of need). Button 1260 can be configured to be utilized in a similar or identical manner to any of the devices described with respect to FIGS. 12-15E herein and/or disclosed in U.S. Provisional Application No. 63/371,339 incorporated by reference herein.

FIG. 5K illustrates a top perspective view of shell 1200c and FIGS. 5L-5M illustrate bottom perspective views of shell 1200c. FIGS. 5K-5L illustrates shell 1200c with diaphragm 1264 connected and FIG. 5M illustrates shell 1200c without the diaphragm 1264.

Shell 1200c can include various structure that can engage with portions of electronics assembly 1200b and/or act to operably position portions of electronics assembly 1200b. For example, shell 1200c can include structure that engages and/or operably positions any or all of circuit boards 1230, 1231 (see FIGS. 5N-5Q). For example, shell 1200c can include stems 1221a, 1221b, 1221c, 1221d that extend outward from (for example, generally perpendicular to) an interior surface 1220 of shell 1200c, and stems 1221a, 1221b, 1221c, 1221d can engage with portions of circuit boards 1230, 1231. For example, a first portion (bottom portion) of stems 1221a, 1221b can be sized and/or shaped to fit within openings 1231a, 1231b of circuit board 1231 (see FIGS. 5K, 5S, 5T, and 5V), and a second portion (top portion) of stems 1221a, 1221b can be sized and/or shaped to extend through openings 1230a, 1230b of circuit board 1230 (see FIGS. 5K, 5S, 5T, and 5U). As another example, a first portion (bottom portion) of stems 1221c, 1221d can be sized and/or shaped to fit within openings 1231c, 1231d of circuit board 1231 (see FIGS. 5K, 5V), and a second portion (top portion) of stems 1221c, 1221d can be sized and/or shaped to extend through openings 1230c, 1230d of circuit board 1230 (see FIGS. 5K, 5U). Shell 1200c can include stems 1227 (which can be shorter than stems 1221a-d) extending outward from (for example, generally perpendicular to) an interior surface 1220 of shell 1200c, and stems 1227 can extend through openings 1231g, 1231h, 1231i, 1231j (see FIGS. 5K, 5V, and 5S-5T). Shell can include stems 1223a, 1223b extending outward from (for example, generally perpendicular to) an interior surface 1220 of shell 1200c, and stems 1223a, 1223b can extend through openings 1231e, 1231f of circuit board 1231 and extend to a position underneath (for example, contacting) circuit board 1230.

As shown in FIGS. 5K-5M, shell 1200c can include openings 1224a, 1224b that are sized and/or shaped to allow probes 1244a, 1244b to extend therethrough. Openings 1224a, 1224b can allow thermally conductive probes 1244a, 1244b to extend through shell 1200c and a housing formed by shells 1200a, 1200c. Such configuration can allow probes 1244a, 1244b to contact substrate 1150 when hub 1200 and dock 1100 are secured together, which can in turn allow probes 1244a, 1244b to receive thermal energy from substrate 1150 (from skin of the user) and transmit such thermal energy to and/or toward temperature sensors 1240a, 1240c as described further below.

As discussed previously, wearable device 1000 can include a button 1222 configured to transition the wearable device 1000 (for example, hub 1200) from a non-operational mode to an operational mode (and vice versa) for example, or carry out other actions. Button 1222 can be coupled to shell 1200c and can include and/or interact with switch 1249 (see FIG. 15Q). In some implementations, when hub 1200 (and wearable device 1000) is in the non-operational mode, electronic functionality of the wearable device 1000 is disabled, for example, wireless communication is not allowed and/or physiological measurements (such as temperature) and/or orientation of a user are not determined. Conversely, in some implementations, when hub 1200 (and wearable device 1000) is in the operational mode, measurements of physiological parameters (such as temperature) and/or orientation of a user are enabled and wireless communication with separate devices is enabled. Button 1222 can be located on a portion of hub 1200 such that button 1222 is inaccessible when hub 1200 and dock 1100 are coupled together. For example, with reference to at least FIGS. 1A-1B, 3A-3B, 4A-4B, and 5D-5E, when hub 1200 and dock 1100 are coupled together, button 1222 can face toward substrate 1150 and can be hidden. Such configurations can advantageously inhibit or prevent the wearable device 1000 from being turned off when the wearable device 1000 is secured to the user's skin and/or when hub 1200 and dock 1100 are connected.

In some implementations, shell 1200c comprises more than one material. In some implementations, shell 1200c includes a first portion 1220a made of a first material and a second portion 1220b made of a second material that is different than the first material. For example, the first portion 1220a can be made of a more rigid material than the second portion 1220b. In some implementations, the second portion 1220b is injection molded onto the first portion 1220a. The second portion 1220b can extend around an opening 1229 of shell 1200c and/or extend around openings 1224a, 1224b of shell 1200c, and/or can form a portion of button 1222 (for example, a portion of button 1222 that includes a protrusion 1225 that can engage switch 1249). Advantageously, in some implementations, the second portion 1220b is configured to form a seal around openings 1229, 1224a, 1224b (for example, a water and/or air tight seal).

FIGS. 5N-5P illustrate top perspective views of electronics assembly 1200b of hub 1200 and FIG. 5Q illustrates a bottom perspective view of electronics assembly 1200b. FIG. 5R illustrates a partially exploded bottom perspective view of electronics assembly 1200b. FIGS. 5S-5T illustrate hub 1200 with shell 1200a removed. FIGS. 5U-5V illustrate top views of circuit boards 1230, 1231 (respectively).

Wearable device 1000 can include circuit boards 1230, 1231 as discussed above. Circuit boards 1230, 1231 can mechanically support and electrically connect various electrical components of the wearable device 1000 to facilitate the performance of various functions of the wearable device 1000. Such electrical components can include without limitation, processor 1001, storage device 1002, communication module 1003, information element 1005, one or more temperature sensors 1006, motion sensor 1010, microphone(s) 1011, and/or other sensor(s) 1012. Processor 1237 can be an implementation of processor 1001 and can be in the form of a chip mounted to circuit board 1230. Motion sensor 1265 can be an implementation of motion sensor 1010 and can be in the form of a chip mounted to circuit board 1230. Microphone 1266, illustrated in FIGS. 5P, 5S, 5V, 5W, and 5X, can be an implementation of microphone 1011 and can be in the form of a chip mounted to circuit board 1231. Microphone 1266 can be used in the digital stethoscope functionality of wearable device 1000 as described herein when included. Microphone 1267 can be another implementation of microphone 1011 and can be in the form of a chip mounted to circuit board 1230. Microphone 1267 can be used to assess vibration of the housing of the wearable device 1000 and/or vibration and/or sounds external to the housing of the wearable device 1000 (e.g., ambient noise). Temperature sensors 1240a, 1240b, 1240c, 1240d can be implementations of temperature sensors 1006. Circuit boards 1230, 1231 can be spaced apart from one another by a gap. In some implementations, circuit board 1230, 331 are oriented parallel (e.g., substantially parallel) to one another. Battery 1232, which can be an implementation of battery 1004 described above, can be positioned between circuit boards 1230, 1231 as shown. Battery 1232 can provide power to the hardware/electrical components of the wearable device 1000 which are described herein. Battery 1232 can be a coin cell battery (such as a lithium coin cell battery). Battery 1232 can have a circular shape. Battery 1232 can comprise a metal housing. Battery 1232 can be in electrical contact with circuit board 1230 and/or circuit board 1231 via one or more electrical contacts. In some implementations, battery 1232 is not rechargeable. In some implementations, circuit boards 1330, 1331 are mechanically and/or electrically coupled with one another via one or more headers 1236, which can facilitate communication between circuit boards 1330, 1331 (e.g., signals communicated therebetween) and/or electrical components mounted thereon. Such headers 1236 can act to maintain the spacing and/or orientation of circuit boards 1330, 1331 with respect to each other.

Wearable device 1000 can include near field communication (NFC) functional capabilities (for example, RFID) that can enable wearable device 1000 to interact and/or communicate with separate computing devices. Such NFC functional capabilities can enable the wearable device 1000 to, among other things: confirm or verify that it is and/or is made up of authentic components; transfer data (for example, orientation and/or physiological data obtained by wearable device 1000); and determine a lifespan of the wearable device 1000. For example, wearable device 1000 can include NFC transponder 1233 (for example, in the form of a chip) that can interact with an RFID reader of a separate computing device that emits a radio frequency. NFC transponder 1233 can be an implementation of and/or be part of communication module 1003 discussed above. NFC transponder 1233 can be positioned within the housing of hub 1200 defined by shells 1200a, 1200c. NFC transponder 1233 can be positioned near an exterior portion of the housing, for example, within socket 1210f discussed above (which also may be referred to as a “cavity”).

With reference to FIGS. 5N-5P, wearable device 1000 can include an antenna 1235 to facilitate wireless communication. Antenna 1235 can be an implementation of and/or be part of communication module 1003 discussed above. Antenna 1235 can allow wearable device 1000 to wirelessly communicate via any of the communication protocols discussed elsewhere herein, such as but not limited to, Wi-Fi (802.11x), Bluetooth®, ZigBee®, Z-wave®, cellular telephony, infrared, near-field communications (NFC), RFID, satellite transmission, proprietary protocols, combinations of the same, and the like. As discussed previously as shown in FIGS. 5N-5P, wearable device 1000 can include a battery 1232, which, in some implementations, is placed between and/or adjacent circuit boards 1230, 1231. In some cases, the battery 1232 comprises a metal housing that can negatively impair antenna range. Advantageously, in some implementations where battery 1232 is positioned adjacent circuit boards 1230, 1231, antenna 1235 is positioned spaced away from the circuit boards 1230, 1231 in order to minimize the affect of the battery 1232 on the range of the antenna 1235. Such configurations can also position the antenna 1235 further away from the user's skin and body when wearable device 1000 is secured to the user, which can also improve antenna range since the body can negatively impair antenna range. For example, in some implementations, the wearable device 1000 includes a frame 1234 coupled to circuit board 1230 and/or 1231 that mounts the antenna 1235 and positions the antenna 1235 away from circuit board 1230, 1231. Antenna 1235 can be positioned and/or secured atop a top surface of frame 1234, as shown. Frame 1234 can include legs 1234a, 1234b, 1234c, 1234d, 1234f, 1234g and protrusions 1234e that can engage openings/notches 1230h, 1230i, 1230j, 1230k of circuit board 1230, as shown. Ends of antenna 1235a, 1235b can be coupled to portions of circuit board 1330 as shown in FIGS. 5N-5O.

As mentioned above, wearable device 1000 can include a status indicator 1009 configured to indicate a status of the wearable device 1000, such as whether the wearable device 1000 is in an operational (“on”) mode, whether the wearable device 1000 is pairing or has paired with a separate device, whether an error has been detected, and/or a power level of the wearable device 1000. Such indicator 1009 can be implemented as emitter 1297, illustrated in FIGS. 5N-5P and mounted to circuit board 1230. Emitter 1297 can be positioned within enclosure 1210d of shell 1200a as discussed above. The emitter 1297 can include one or more light-emitting diodes (LEDs). The emitter 1297 can emit light of certain colors to indicate certain statuses of the wearable device 1000. For example, the emitter 1297 can emit a green light to indicate that the wearable device 1000 is powered “on” or a red light to indicate the wearable device 1000 is “off”. A housing formed by shells 1200a, 1200c can include an opening configured to allow light emitted from the emitter 1297 to be visible from a location outside an interior of the housing. For example, as discussed above, shell 1200a can include hole 1215. Additionally or alternatively, shells 1200a and/or 1200c can comprise a transparent or semi-transparent material that allows light emitted from the emitter 1297 to be seen from a location outside an interior of the housing. In some implementations, hole 1215 is at least partially aligned with emitter 1297 to allow light emitted from the emitter 1297 to more easily pass through the housing.

With continued reference to FIGS. 5N-5P, wearable device 1000 can include one or more or a plurality of emitters 1299 that can be utilized to illuminate a display of wearable device 1000, such as display 1262 (which can be an implementation of display 1007). Emitters 1299 (which can include one, two, three, four, five, six, seven, eight, nine, or ten or more emitters 1299) can be arranged to correspond to a shape and/or size of display 1262. For example, where display 1262 comprises an arch shape, emitters 1299 can be mounted to circuit board 1230 and arranged in an arch pattern. The display 1262 can comprise a light pipe 1210 as shown in FIGS. 5I-5J. The light pipe 1210 can comprise one or more or a plurality of prisms 1210a also shown in FIGS. 5I-5J. Prisms 1210a (which can include one, two, three, four, five, six, seven, eight, nine, or ten or more prisms 1210a) can be arranged to correspond to a shape and/or size of display 1262. For example, where display 1262 comprises an arch shape, prisms 1210a can be arranged in an arch pattern in light pipe 1210. Furthermore, prisms 1210a can correspond with emitters 1299 (e.g., in number and/or arrangement). Prisms 1210a can be aligned or offset (e.g., vertically) from emitters 1299 or otherwise positioned such that light emitted from emitters 1299 passes through prisms 1210a. In some implementations, emitters 1299 and prisms 1210a of light pipe 1210 cooperate to generate display 1262 and/or one or more display elements thereof (such as display elements 1262a, 1262b, 1262c shown in FIG. 5AA).

As discussed herein, wearable device 1000 can include one or more temperature sensors that can be mounted to circuit boards 1230, 1231. As shown in at least FIGS. 5N-5P, wearable device 1000 can include temperature sensors 1240b and 1240d mounted to circuit board 1230 and temperature sensors 1240a and 1240c mounted to circuit board 1331. In some implementations, circuit boards 1230, 1231 and/or portions thereof can be configured to inhibit or minimize heat flow therealong and/or between various hardware/electrical components of wearable device 1000. Such configuration can allow temperature sensors 1240a, 1240b, 1240c, 1240d to be utilized to capture unique temperature values which can be advantageous in determining body temperature of a user. In some implementations, circuit boards 1230, 1231 and/or portions of thereof can be configured to transmit heat flow therealong and/or between various hardware/electrical components of wearable device 1000. Such configuration can be advantageous in determining body temperature of a user.

With reference to at least FIGS. 5N-5R, wearable device 1000 can include thermally conductive probes 1244a, 1244b. Thermally conductive probes 1244a, 1244b can advantageously help transmit thermal energy to and/or toward temperature sensors 1240a, 1240c, as also discussed elsewhere herein. Thermally conductive probes 1244a, 1244b can be rigid. Thermally conductive probes 1244a, 1244b can comprise a metallic material (for example, comprising brass and/or aluminum). Thermally conductive probes 1244a, 1244b can comprise a circular cross-section. Thermally conductive probes 1244a, 1244b can have a first end that is substantially flat and a second end that is tapered (see FIG. 5R). Thermally conductive probes 1244a, 1244b, temperature sensors 1240a, 1240b, 1240c, 1240d, circuit boards 1230, 1231, and/or their configuration relative to one another (in addition to any thermally conductive element between any one or more of the above) can be similar or identical to the probes, temperature sensors, circuit boards, and thermally conductive element described in U.S. Pat Pub. No. US2023/0087671 incorporated by reference herein. For example, probe 1244a can be substantially aligned with temperature sensor 1240a and/or 1240b, and probe 1244b can be substantially aligned with temperature sensor 1240c and/or 1240d.

Thermally conductive probe 1244a can have a first end positioned adjacent and/or secured to a portion of circuit board 1231 and a second end opposite such first end. In some implementations, thermally conductive probe 1244a (for example, such first end thereof) is soldered to circuit board 1231. Such first end of thermally conductive probe 1244a can be positioned adjacent and/or secured to a portion of circuit board 1231 such that probe 1244a is substantially aligned with one or both of temperature sensors 1240a, 1240b (see FIGS. 5O and 5P). Thermally conductive probe 1244b can have a first end positioned adjacent and/or secured to a portion of circuit board 1231 and a second end opposite such first end. In some implementations, thermally conductive probe 1244b (for example, such first end thereof) is soldered to circuit board 1231. Such first end of thermally conductive probe 1244b can be positioned adjacent and/or secured to a portion of circuit board 1231 such that probe 1244b is substantially aligned with one or both of temperature sensors 1240c, 1240d (see FIG. 5N).

Circuit board 1231 can include one or more openings configured to allow thermal energy from probes 1244a, 1244b to pass through circuit board 1231 and to temperature sensors 1240a, 1240c. The position of such one or more openings is shown in FIG. 5Y as 1241a, 1241c. For example, circuit board 1231 can include holes similar or identical to holes 348a, 348b that can extend through circuit board 331 as described in U.S. Pat Pub. No. US2023/0087671 incorporated by reference herein. Such holes in circuit board 1231 can be arranged at the location indicated with arrows 1277a, 1277b in FIG. 5R, for example. The number, arrangement, and/or configuration of such holes in circuit board 1231 can be similar or identical to the number, arrangement, and/or configuration of holes 348a, 348b described in U.S. Pat Pub. No. US2023/0087671 incorporated by reference herein. Such holes in circuit board 1231 (which can be arranged at the location indicated with arrows 1277a, 1277b in FIG. 5R) can be: arranged in an array and/or group proximate one another and can be positioned between thermally conductive probe 1244a, 1244b and temperature sensor 1240a, 1240c; can include one or a plurality of such holes; and/or can be filled with a thermally conductive material or not filled with any material.

Circuit board 1230 can include one or more openings configured to allow thermal energy to pass through circuit board 1230 and to temperature sensors 1240b, 1240d. The position of such one or more openings is shown in FIG. 5Y as 1241b, 1241d. For example, circuit board 1230 can include holes similar or identical to holes 350a, 350b that can extend through circuit board 1231 as described in U.S. Pat Pub. No. US2023/0087671 incorporated by reference herein. Such holes in circuit board 1230 can be arranged at the location indicated with arrows 1277c, 1277d in FIG. 5R, for example. The number, arrangement, and/or configuration of such holes in circuit board 1230 can be similar or identical to the number, arrangement, and/or configuration of holes 350a, 350b described in U.S. Pat Pub. No. US2023/0087671 incorporated by reference herein. Circuit board 1230 can include holes (which can be arranged at the location indicated with arrow 1277c in FIG. 5R) that can be: arranged in an array and/or group proximate one another and can be positioned between a portion of thermally conductive element 1242 (for example, at or near a portion 1242a of element 1242) and temperature sensor 1240d; can include one or a plurality of such holes; and/or can be filled with a thermally conductive material or not filled with any material. Circuit board 1230 can include holes (which can be arranged at the location indicated with arrow 1277d in FIG. 12Q) that can be: arranged in an array and/or group proximate one another and can be positioned so as to be substantially aligned with an axis that extends through temperature sensors 1240a, 1240b; can include one or a plurality of such holes; and/or can be filled with a thermally conductive material or not filled with any material.

Thermally conductive probes 1244a, 1244b can be configured to contact substrate 1150 when wearable device 1000 is in use and/or when hub 1200 and dock 1100 are coupled together. As discussed above, hub 1200 (e.g, shell 1200c) can include openings 1224a, 1224b configured to allow probes 1244a, 1244b to extend through a housing (defined by shells 1200a, 1200c) and contact substrate 1150 when wearable device 1000 is in use and/or when hub 1200 and dock 1100 are coupled together.

Temperature sensors 1240a, 1240b, 1240c, 1240d can be configured to generate one or more signals responsive to detected thermal energy, determine temperature, and/or transmit such generated one or more signals and/or such determined temperature to the processor 1001 (e.g., processor 1237) of the wearable device 1000 continuously and/or intermittently. For example, temperature sensors 1240a, 1240b, 1240c, 1240d can be configured to generate one or more signals responsive to detected thermal energy, determine temperature, and/or transmit such generated one or more signals and/or such determined temperature every 0.5 seconds, 1 second, 2 second, 3 seconds, 4 seconds, 5 seconds, 10 seconds, 30 seconds, 1 minute, 2 minute, 3 minutes, 4 minutes, 5 minutes, or at other intervals. Such generated one or more signals, determined temperature, and/or transmission of such generated one or more signals and/or determined temperature for each of temperature sensors 1240a, 1240b, 1240c, 1240d can be simultaneous or non-simultaneous.

Wearable device 1000 can be used to measure a user's temperature over time. As discussed above, wearable device 1000 can be configured to wirelessly communicate with) a separate computing device, such as a patient monitor and/or a mobile device (e.g., smart phone). The wearable device 1000 can wirelessly transmit physiological data (such as temperature data) over time (continuously or periodically) to such separate computing device for display, among other things. As also discussed above, wearable device 1000 can wirelessly transmit processed or unprocessed obtained physiological information to a mobile phone (for example) which can include one or more hardware processors configured to execute an application that generates a graphical user interface displaying information representative of the processed or unprocessed physiological information obtained from the wearable device 1000. Such graphical user interfaces can display continuous and/or periodic measurements obtained from the wearable device 1000, display and/or issue various types of alerts, display physiological trend information (for example, temperature trends), among other things. Features or aspects displayed by such graphical user interfaces can include, without limitation, a splash screen, onboarding, device setup, instructions (for example, both visual/graphical and textual) for securing the wearable device 1000 to a user and/or pairing wearable device 1000 to the separate computing device, temperature data and/or trending dashboard, user scenarios, notes (such as medication notes and reminders as well as other user activity notes), temperature trending data and information, user settings and profiles, app settings, and alerts and push notifications.

Temperature sensors 1240b, 1240d can be mounted to a first surface of circuit board 1230 and spaced away from each other. A second surface of circuit board 1230 that is opposite the first surface of the circuit board 1231 can face toward temperature sensors 1240a, 1240c and toward circuit board 1231 (for example, toward a first surface of circuit board 1231 that temperature sensors 1240a, 1240c are mounted to). Circuit board 1230 (for example, second surface of circuit board 1230 that is opposite a first surface of circuit board 1230 that temperature sensor 1240b, 1240d are mounted to) can be spaced from temperature sensor 1240a and/or temperature sensor 1240c by a distance that can be approximately 0.5 mm, approximately 1 mm, approximately 1.5 mm, approximately 2 mm, approximately 2.5 mm, approximately 3 mm, approximately 3.5 mm, or approximately 4 mm, any value or range between any of these values, any value or range bounded by any combination of these values, at least approximately 0.1 mm, at least approximately 0.2 mm, at least approximately 0.3 mm, at least approximately 0.4 mm, at least approximately 0.5 mm, at least approximately 1 mm, at least approximately 1.5 mm, at least approximately 2 mm, at least approximately 2.5 mm, at least approximately 3 mm, at least approximately 3.5 mm, or at least approximately 4 mm.

Temperature sensors 1240a, 1240b, and probe 1244a can be substantially aligned with one another. Similarly, temperature sensor 1240c, 1240d, and probe 1244b can be substantially aligned with one another. Temperature sensors 1240a, 1240b can be thermally insulated from one another. For example, with reference to at least FIGS. 5N-5P, an air gap can be present at least partially between temperature sensor 1240a and 1240b. For example, an air gap can be positioned at least partially between temperature sensor 1240a, a second/bottom surface of circuit board 1230, and temperature sensor 1240b. In some variants, a thermally insulative material is positioned in place of such air gap.

Temperature sensors 1240c, 1240d can be thermally coupled to one another, for example, by a thermally conductive element 1242. Thermally conductive element 1242 can be positioned at least partially between temperature sensors 1240c, 1240d. For example, thermally conductive element 1242 can be positioned between temperature sensor 1240c and a second surface of circuit board 1230 that is opposite a first surface of circuit board 1230 to which temperature sensor 1240d is mounted. Thermally conductive element 1242 can also be positioned between holes extending through circuit board 1230 (which can be at location 1277c in FIG. 5R) and temperature sensor 1240c. Thermally conductive element 1242 can comprise a first end 1242a secured to the second surface of circuit board 1230 (for example, adjacent to holes positioned at location 1277c in FIG. 5R), a second end 1242c secured to the temperature sensor 1240c, and a stem 1242b positioned in between the first and second ends 1242a, 1242c. Thermally conductive element 1242 can comprise a rigid or semi-rigid material. Thermally conductive element 1242 can be in a flexed configuration where stem 1242b is at least partially bent when assembled. Thermally conductive element 1242 can comprise a metallic material, such as copper. As another example, the thermally conductive element 1242 can comprise beryllium copper (BeCu). In some implementations, a thermal material (such as a thermal paste) is positioned between end 1242c and temperature sensor 1240c, which can advantageously increase thermal transmissivity in some cases. Such thermal paste can comprise zinc oxide and/or can be silicone free. Thermally conductive element 1242 can be similar or identical to thermally conductive element 342 described in U.S. Pat Pub. No. US2023/0087671 incorporated by reference herein (for example, with respect to dimensions thereof).

FIG. 5Y illustrates a cross-section taken through wearable device 1000 when the wearable device 1000 is secured to user's skin. It is often difficult to accurately estimate internal body temperature based on temperature measurements obtained via skin. Advantageously, the arrangement of temperature sensors 1240a, 1240b, 1240c, 1240d along with various other components of the wearable device 1000 disclosed herein can facilitate more robust determinations of internal body temperature.

As discussed previously, wearable device 1000 can include a substrate 1150 that can be positioned to contact and/or secure to skin of a user when wearable device 1000 is in use. Wearable device 1000 can be secured to the skin via securement of dock 1100 (and substrate 1150) to the skin, prior to, during, and/or after securement of hub 1200 to dock 1100 which is described elsewhere herein. As also described previously, thermally conductive probes 1244a, 1244b can extend through opening 1132 in frame 1130 of dock 1100 and contact substrate 1150 (for example, an interior surface of substrate 1150 that is opposite to an exterior or skin-facing surface of substrate 1150) when hub 1200 and dock 1100 are coupled together. In some cases, probes 1244a, 1244b cause substrate 1150 to “bulge”, as shown in FIG. 5Y (which may be an exaggerated representation of such “bulging”), for example, due to the length of probes 1244a, 1244b in relation to dimensions of the hub 1200 and/or dock 1100 (and/or portions thereof). Such bulging may cause corresponding pressure and/or “bulging” of a portion of the user's skin underneath (see FIG. 5Y). In some implementations, probes 1244a, 1244b are not configured to cause such “bulging”, but merely contact substrate 1150. In some implementations, none of temperature sensors 1240a, 1240b, 1240c, 1240d and none of thermally conductive probes 1244a, 1244b contact skin of the user when the wearable device 1000 is in use. When wearable device 1000 is in use, probes 1244a, 1244b can receive thermal energy via contact with substrate 1150 which itself contacts and receives thermal energy from skin. Such configurations can provide more consistent temperature readings since moisture and/or other characteristics of skin (for example, oil or dirt levels on skin) may result in inconsistent temperature readings.

Thermal energy radiating from the internal body of the user passing through the skin can be conducted through substrate 1150 and through thermally conductive probes 1244a, 1244b. Thermally conductive probes 1244a, 1244b can act as a thermal conduit to transmit thermal energy toward temperature sensors 1240a, 1240c. As discussed above, circuit board 1231 can include holes that can allow such thermal energy to pass through circuit board 1231 to temperature sensors 1240a, 1240c.

In addition to temperature sensors 1240a, 1240c, wearable device 1000 can include temperature sensors 1240b, 1240d. Temperature sensors 1240b, 1240d can be operably positioned within a housing defined by shells 1200a, 1200c to be positioned farther away from the user's skin than temperature sensors 1240a, 1240c when wearable device 1000 is secured to the user. For example, temperature sensors 1240b, 1240d can be positioned on a surface of circuit board 1230 that faces toward a top interior surface of shell 1200a. Such arrangement allows temperature sensors 1240b, 1240d to be more responsive to ambient temperature (for example, environmental temperature outside the housing of wearable device 1000). In some variants, thermal putty (for example, a ceramic filled silicone sheet) is positioned between temperature sensors 1240b, 1240d and the top interior surface of shell 1200a in order to provide better thermal contact between temperature sensors 1240b, 1240d and ambient.

As discussed herein, an air gap can be positioned between temperature sensor 1240a and temperature sensor 1240b (for example, between circuit board 1230 and temperature sensor 1240a). As also discussed above, a thermally conductive element 1242 (only partially shown in the cross-section of FIG. 5Y) can be positioned between temperature sensors 1240c, 1240d. In such configurations, and similar to as described with respect to wearable device 100 in U.S. Pat Pub. No. US2023/0087671 incorporated by reference herein, two unique temperature gradients can be established in wearable device 1000, one of which is between temperature sensors 1240a, 1240b (which can have an air gap between), and the other of which is between temperature sensors 1240c, 1240d (which can have a thermally conductive element 1242 between). Temperature data from each of temperature sensors 1240a, 1240b, 1240c, 1240d can advantageously be utilized by wearable device 1000 in a similar or identical manner as that described with respect to wearable device 100 in U.S. Pat Pub. No. US2023/0087671 incorporated by reference herein; which can be to facilitate more robust approximations of internal body temperature. For example, temperature data from temperature sensors 1240a and 1240b can be compared (for example, differences can be determined therebetween) and/or temperature sensors 1240c and 1240d can be compared (for example, differences can be determined therebetween). Additionally or alternatively, comparisons between temperature data from temperature sensors 1240a, 1240c and/or between temperature sensors 1240b, 1240d can be made (for example, differences therebetween can be determined). Additionally or alternatively, comparisons between temperature data from temperature sensors 1240a, 1240d and/or between temperature sensors 1240b, 1240c can be made (for example, differences therebetween can be determined). Additionally, known information relating to thermal properties of air (which can be present between temperature sensors 1240a, 1240b as discussed previously) and/or thermally conductive element 1242 can be utilized along with temperature data and/or comparisons of temperature data from temperature sensors 1240a, 1240b, 1240c, 1240d to determine robust approximations of internal body temperature. Such information can advantageously be utilized to overcome challenges of estimating internal body temperature based on skin temperature readings. Wearable device 1000 (for example, processor 1001/1237) can determine body temperature values based on any of such above-described comparisons and/or differences and/or other information.

FIGS. 5W-5X illustrate cross-sections taken through hub 1200 at the locations indicated in FIGS. 5C-5D. In some implementations, wearable device 1000 is configured to monitor body sounds of a user. Such body sounds can include cardiac activity, lung activity, snoring, wheezing, coughing, choking, and/or breathing of the user and/or the like. Wearable device 1000 can comprise a digital stethoscope for this purpose. As shown in FIGS. 5W-5X, wearable device 1000 can include a diaphragm 1264 configured to move (for example, vibrate) responsive to such body sounds (e.g., cardiac and/or lung activity) of a user to which the wearable device 1000 is attached. Diaphragm 1264 can be coupled to shell 1200c as mentioned previously. Movement of diaphragm 1264 (for example, vibration) can generate sound wave(s) within an interior portion of wearable device 1000, such as interior portion 1275 identified in FIGS. 5W-5X. Interior portion 1275 (which can also be referred to as a “sound cavity”) can be within a housing of wearable device 1000, and such housing can be defined by shells 1200a, 1200c. Such interior portion 1275 can be defined at least partially between diaphragm 1264, a portion of circuit board 1231, and/or portions of shell 1200c, for example. Sound wave(s) generated by diaphragm 1264 within interior portion 1275 (responsive to body sounds of a user) can be detected by microphone 1266. In some implementations, microphone 1266 is positioned on a first surface of circuit board 1231 that is opposite to a second surface of circuit board 1231 that defines and/or is positioned adjacent the interior portion 1275. In some of such implementations, circuit board 1231 includes one or more holes configured to allow sound wave(s) generated by diaphragm 1264 to pass through circuit board 1231 and to microphone 1266. For example, circuit board 1231 can include a hole 1231k that is positioned adjacent microphone 1266. In some implementations, hole 1231k is not aligned with an axis extending through microphone 1266. Microphone 1266 can detect the above-described sound wave(s) and generate one or more signals based on the detected sound wave(s). Such one or more signals can, in turn, be received by one or more processors of the wearable device 1000 (for example, processor 1001/1237) for determination of body sounds including cardiac function and/or lung function of the user. In some implementations, the interior portion 1275 is substantially sealed. For example, with reference to FIGS. 5K-5M and FIGS. 5W-5X, a portion of shell 1200c (such as portion 1220b) can seal against a portion of circuit board 1231 around hole 1231k and diaphragm 1264 can be sealingly coupled to shell 1200c around opening 1229 of shell 1200c. Such sealing arrangement can advantageously increase the quality of sound detection by microphone 1266 and therefore, allow for improved body sound monitoring (which can include cardiac and/or lung activity/function monitoring) by wearable device 1000. In some implementations, wearable device 1000 can include a microphone 1267 as described herein configured to detect vibrations of the housing (e.g., of clothing of a user rubbing against the housing) and/or ambient noise/sound waves (e.g., sounds external to the housing) and generate one or more signals based on such vibrations/sound waves. Such one or more signals can, in turn, be received by one or more processors of the wearable device 1000 (for example, processor 1001/1237) for determination of ambient noise. Furthermore, the one or more processors of the wearable device can determine at least one of a corrected cardiac measurement and a corrected lung measurement responsive to said signals from microphones 1266 and 1267 (e.g., background subtracted measurements). When a microphone 1267 is included in wearable device 1000, it can be positioned further away from the user than the microphone 1266 when the wearable device 1000 is in use. For example, the microphone 1267 can be mounted to circuit board 1230 as shown in at least FIG. 5U.

FIG. 5Y and FIG. 5Z illustrate cross-sectional views through the wearable device 1000 as identified in FIG. 1D attached to a user. FIG. 5Y shows in particular a cross-section through probes 1244a, 1244b and how they can interact with substrate 1150 and the skin of the user. As shown, the wearable device 1000 can be configured such that probes 1244a, 1244b apply pressure to the substrate 1150 and thus the user's skin. Such a configuration can aid in the thermal coupling of the probes 1244a, 1244b to the user and thus improve a body temperature determination of the user by the wearable device 1000.

As shown in FIG. 5Y, the probes 1244a, 1244b can in some implementations extend a distance D1 below an exterior surface (e.g., bottom) of the hub 1200a. Further as shown, the probes 1244a, 1244b can in some implementations extend a distance D2 below an exterior surface (e.g., bottom) of the frame 1130 of the dock 1100. Distances D1 and D2 can be approximately 0.1 mm, approximately 0.2 mm, approximately 0.3 mm, approximately 0.4 mm, 0.5 mm, approximately 1 mm, approximately 1.5 mm, approximately 2 mm, approximately 2.5 mm, approximately 3 mm, approximately 3.5 mm, or approximately 4 mm, any value or range between any of these values, any value or range bounded by any combination of these values, at least approximately 0.1 mm, at least approximately 0.2 mm, at least approximately 0.3 mm, at least approximately 0.4 mm, at least approximately 0.5 mm, at least approximately 1 mm, at least approximately 1.5 mm, at least approximately 2 mm, at least approximately 2.5 mm, at least approximately 3 mm, at least approximately 3.5 mm, or at least approximately 4 mm. In some implementations, the probes 1244a, 1244b are configured to sit flush or substantially flush with an exterior surface of the hub 1200a and/or an exterior surface of the frame 1130. In some implementations (not shown), substrate 1150 can comprise an openings configured to allow probes 1244a, 1244b to pass therethrough and contact the user's skin directly. Further as shown, the hub 1200 of the wearable device 1000 can have a width W2, and the dock 1100 of the wearable device 1000 can have a width that extends past the width W2 on either side of the hub 1200 of W1. Width W1 can be approximately 3 mm, approximately 4 mm, approximately 5 mm, approximately 5.5 mm, approximately 6 mm, approximately 6.5 mm, approximately 7 mm, approximately 7.5 mm, approximately 8 mm, approximately 8.5 mm, approximately 9 mm, approximately 9.5 mm, approximately 10 mm, approximately 11 mm, approximately 12 mm, approximately 13 mm, any value or range between any of these values, any value or range bounded by any combination of these values, or at least approximately 3 mm. Width W2 can be approximately 10 mm, approximately 15 mm, approximately 20 mm, approximately 25 mm, approximately 30 mm, approximately 35 mm, approximately 40 mm, approximately 45 mm, approximately 50 mm, approximately 55 mm, approximately 60 mm, any value or range between any of these values, any value or range bounded by any combination of these values, or at least approximately 10 mm.

FIG. 5Z shows in particular a cross-section through diaphragm 1264 and how it can interact with substrate 1150 and the skin of the user. As shown, in some implementations the wearable device 1000 can be configured such that the diaphragm 1264 applies pressure to the substrate 1150 and thus the user's skin. Such a configuration can aid in the acoustic coupling of the diaphragm 1264 to the user and thus improve body sound determination by the wearable device 1000 (e.g., improve function of the digital stethoscope formed at least in part by the diaphragm 1264).

As shown in FIG. 5Z, the diaphragm 1264 can in some implementations extend a distance D3 below an exterior surface (e.g., bottom) of the hub 1200a. Further as shown, the diaphragm 1264 can in some implementations extend a distance D4 below an exterior surface (e.g., bottom) of the frame 1130 of the dock 1100. Distances D3 and D4 can be approximately 0.1 mm, approximately 0.2 mm, approximately 0.3 mm, approximately 0.4 mm, 0.5 mm, approximately 1 mm, approximately 1.5 mm, approximately 2 mm, approximately 2.5 mm, approximately 3 mm, approximately 3.5 mm, or approximately 4 mm, any value or range between any of these values, any value or range bounded by any combination of these values, at least approximately 0.1 mm, at least approximately 0.2 mm, at least approximately 0.3 mm, at least approximately 0.4 mm, at least approximately 0.5 mm, at least approximately 1 mm, at least approximately 1.5 mm, at least approximately 2 mm, at least approximately 2.5 mm, at least approximately 3 mm, at least approximately 3.5 mm, or at least approximately 4 mm. In some implementations, the diaphragm 1264 is configured to sit flush or substantially flush with an exterior surface of the hub 1200a and/or an exterior surface of the frame 1130. In some implementations (not shown), substrate 1150 can comprise an opening configured to allow diaphragm 1264 to pass therethrough and contact the user's skin directly. In some implementations, the substrate 1150 can be modified (e.g., heat pressed) to increase an acoustic conductivity thereof. Further as shown, the hub 1200 of the wearable device 1000 can have a length W4, and the dock 1100 of the wearable device 1000 can have a length that extends past the length W4 on either side of the hub 1200 of W3. Length W3 can be approximately 3 mm, approximately 4 mm, approximately 5 mm, approximately 5.5 mm, approximately 6 mm, approximately 6.5 mm, approximately 7 mm, approximately 7.5 mm, approximately 8 mm, approximately 8.5 mm, approximately 9 mm, approximately 9.5 mm, approximately 10 mm, approximately 11 mm, approximately 12 mm, approximately 13 mm, any value or range between any of these values, any value or range bounded by any combination of these values, or at least approximately 3 mm. Length W4 can be approximately 10 mm, approximately 15 mm, approximately 20 mm, approximately 25 mm, approximately 30 mm, approximately 35 mm, approximately 40 mm, approximately 45 mm, approximately 50 mm, approximately 55 mm, approximately 60 mm, any value or range between any of these values, any value or range bounded by any combination of these values, or at least approximately 10 mm.

With continued reference to FIG. 5Z and in relation to the digital stethoscope functionality of wearable device 1000 in some implementations, substrate 1150 can be configured to attach to the skin of the user and move with the skin/body of the user. In other words, substrate 1150 can be closely coupled with the skin of the user and/or act as an extension of the skin of the user. Furthermore, substrate 1150 can be non-dampening (e.g., non-sound dampening). Substrate 1150 can be, for example, a 3M 2480 material with silicone adhesive and non-woven spun lace liner. As mentioned herein, in some implementations the substrate 1150 can be modified where in contact with the diaphragm 1264 to improve its sound transmission properties. For example, a hole can be made in substrate 1150 so that the diaphragm 1264 can touch/couple directly to the skin of the user. As another example, the substrate 1150 can be heat pressed where in contact with the diaphragm 1264.

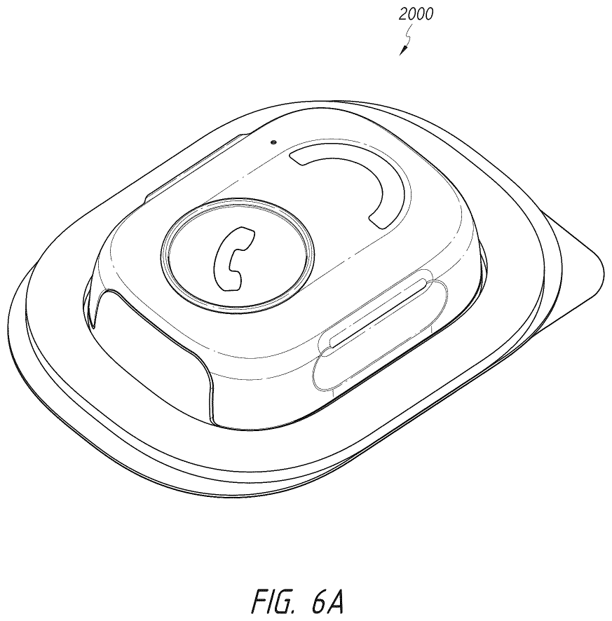

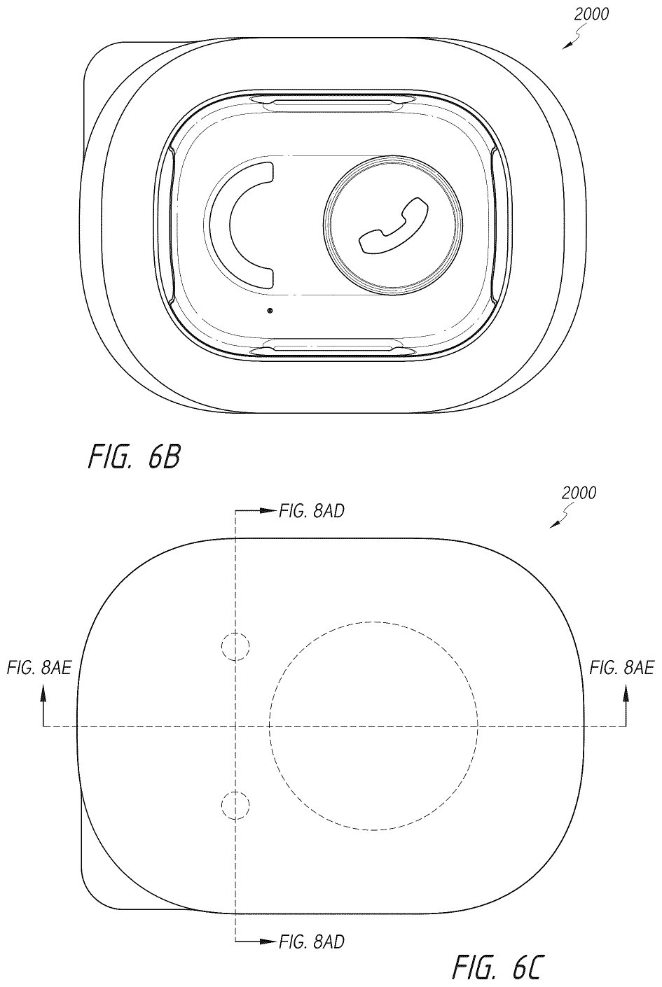

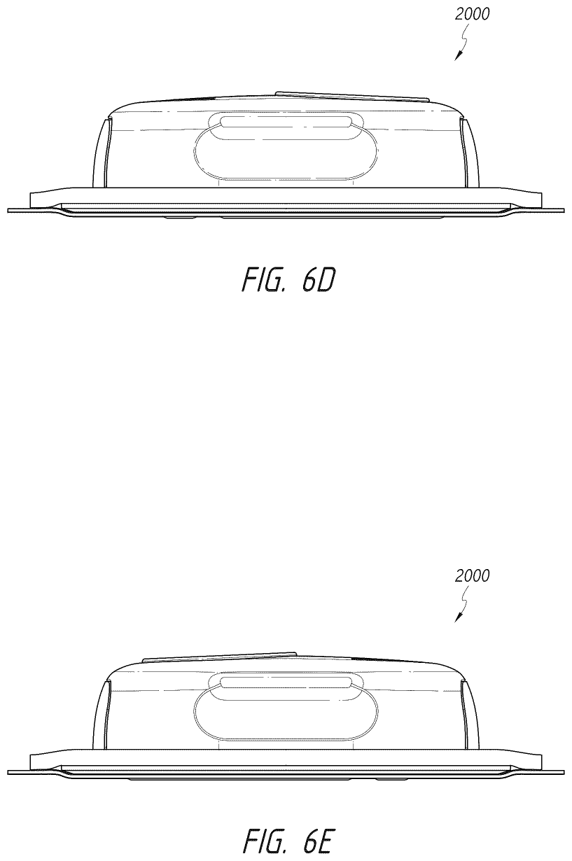

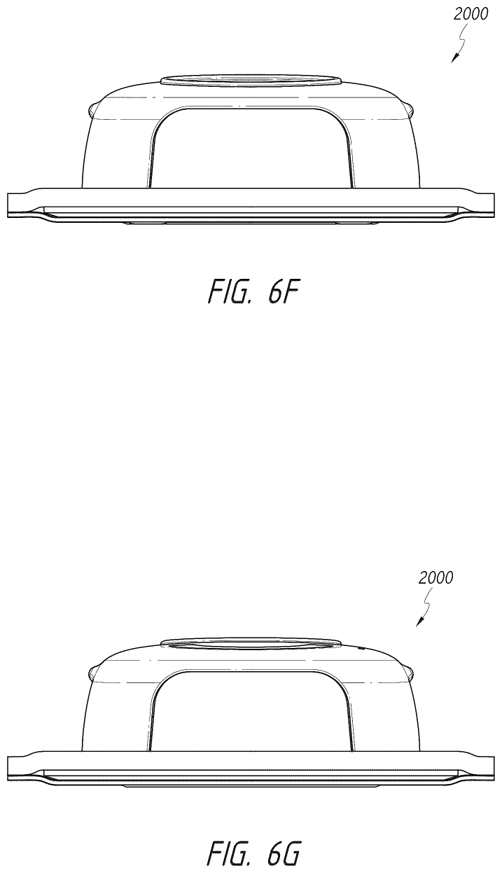

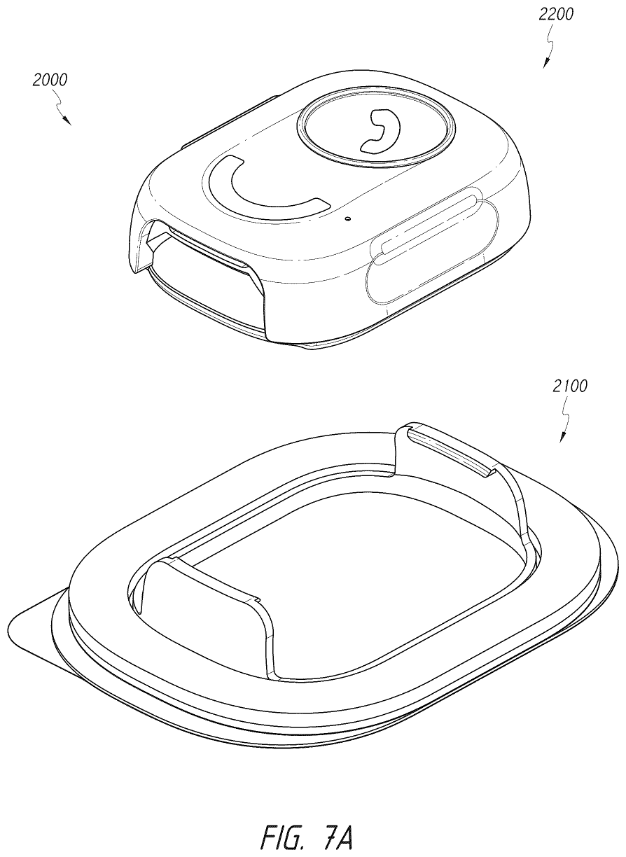

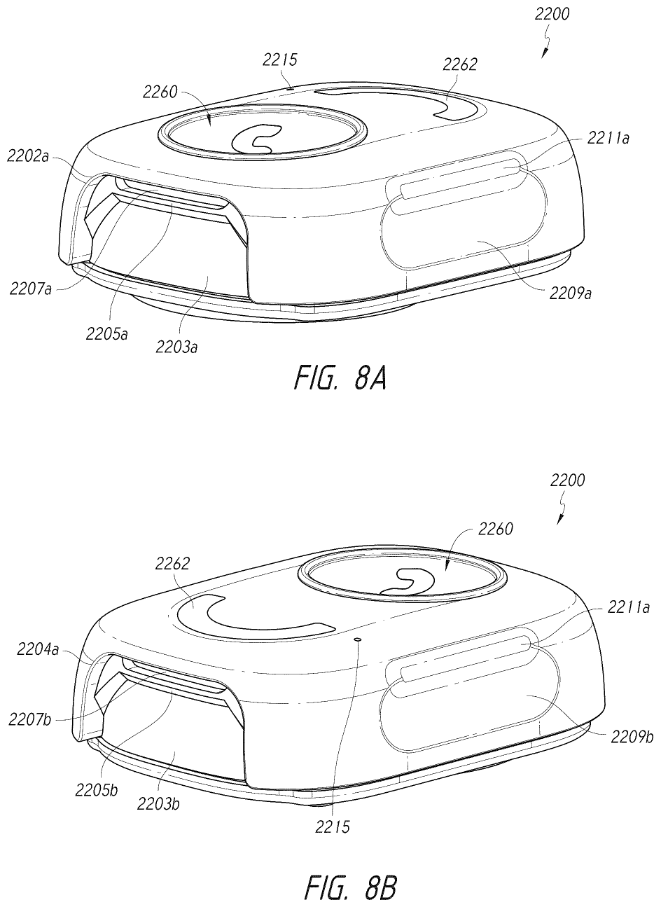

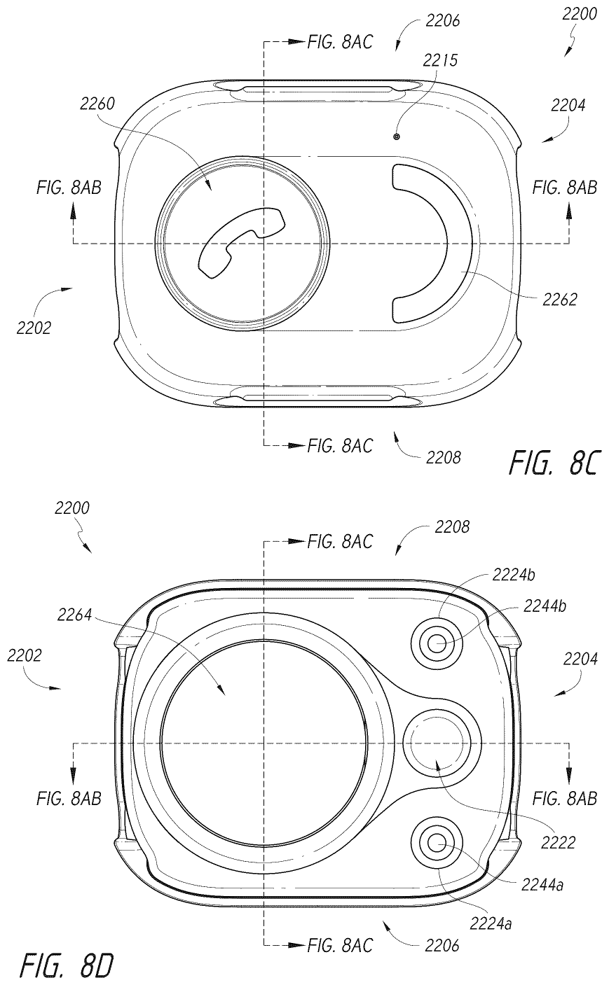

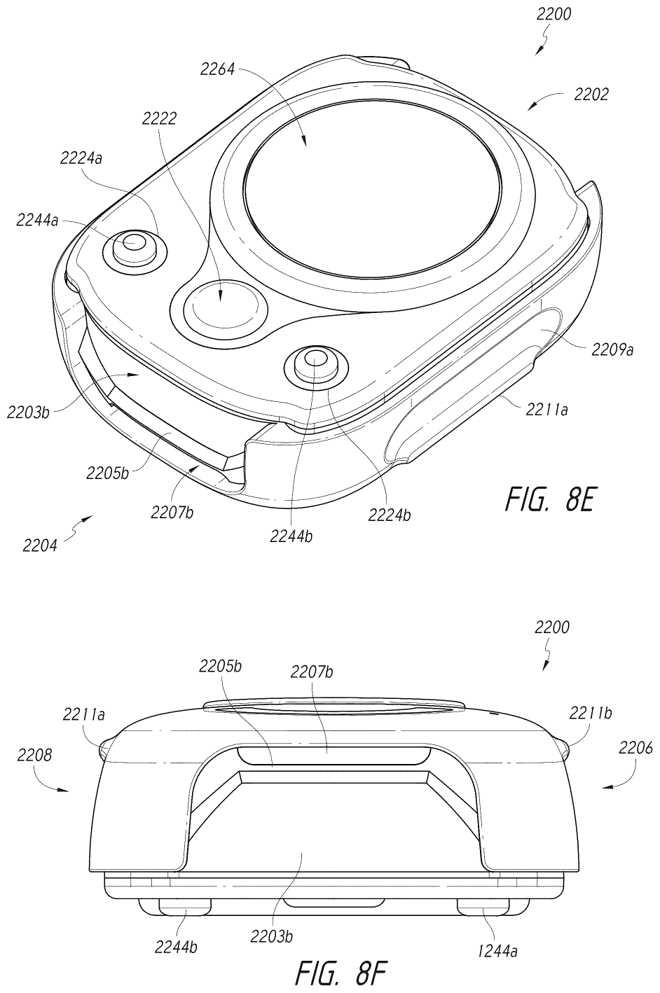

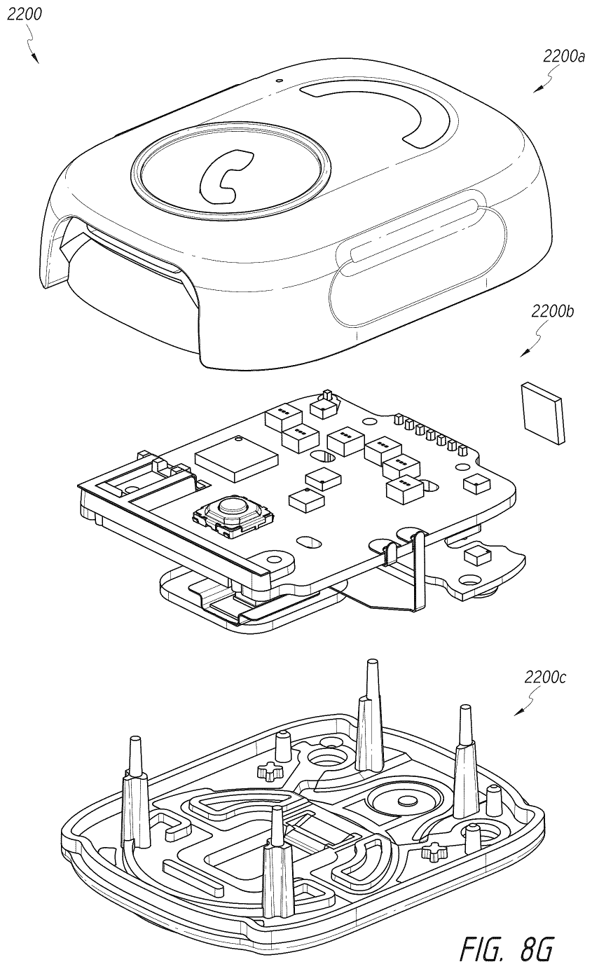

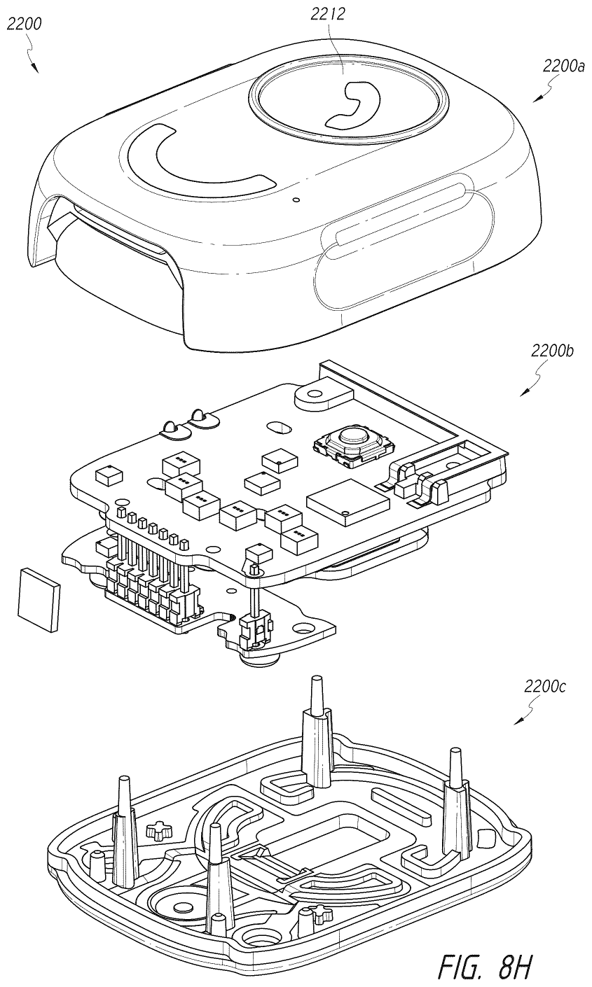

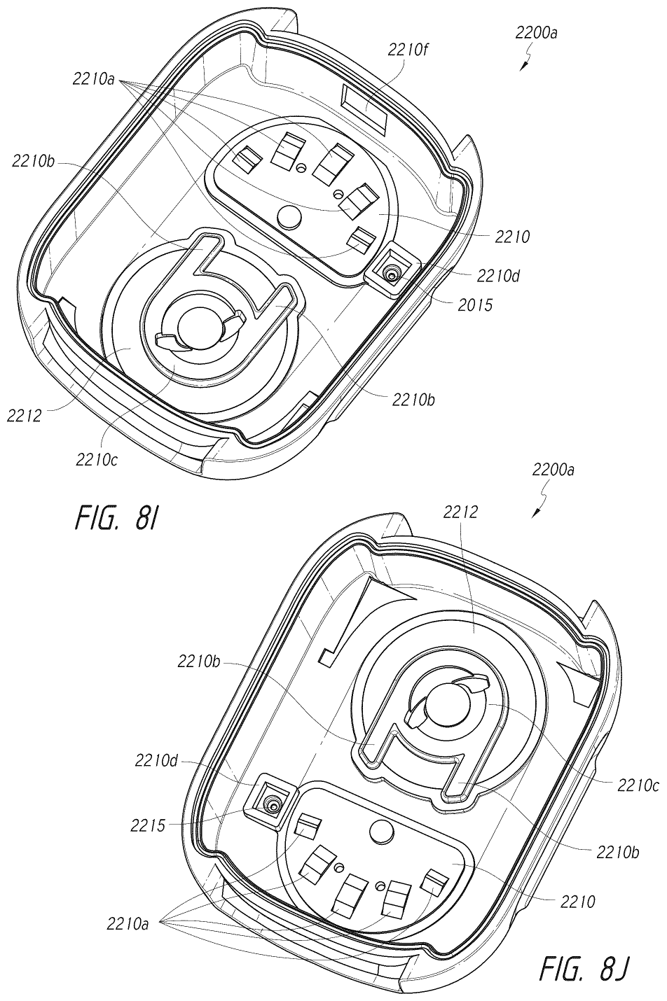

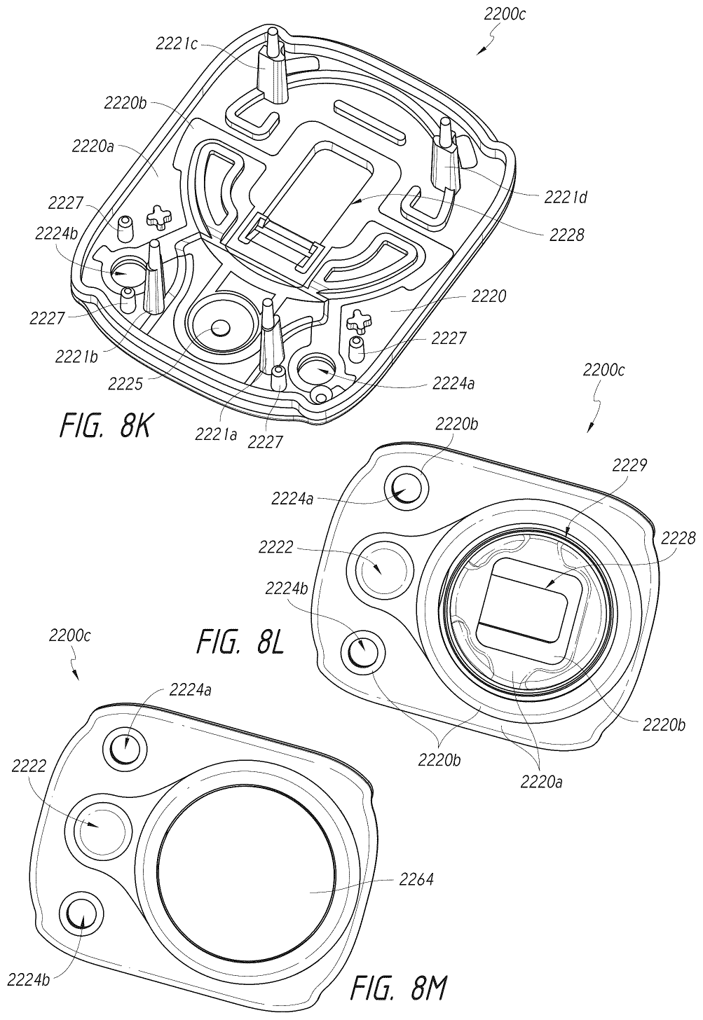

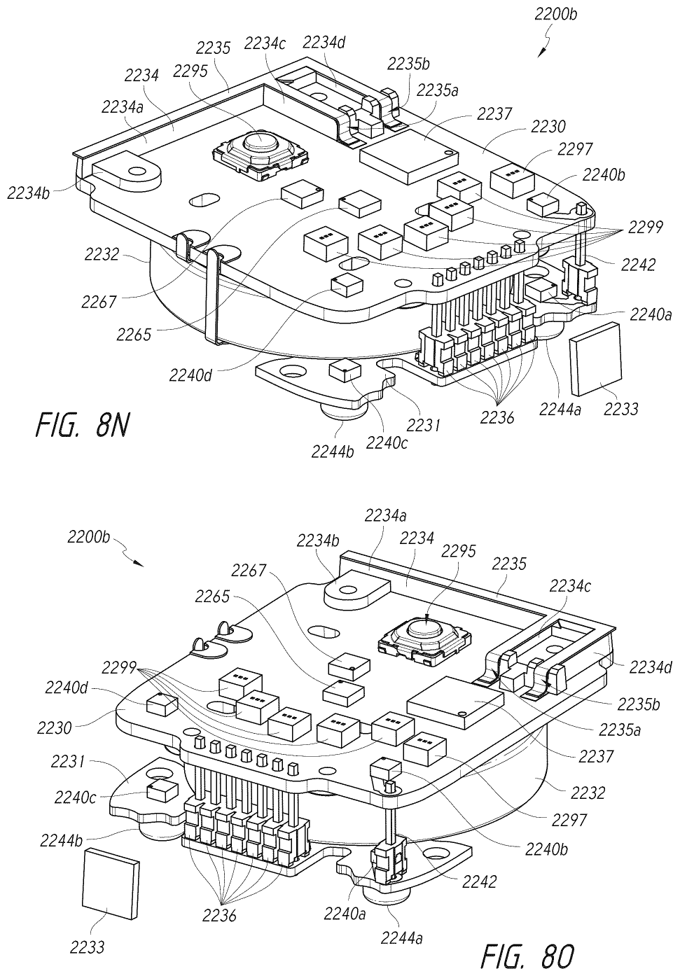

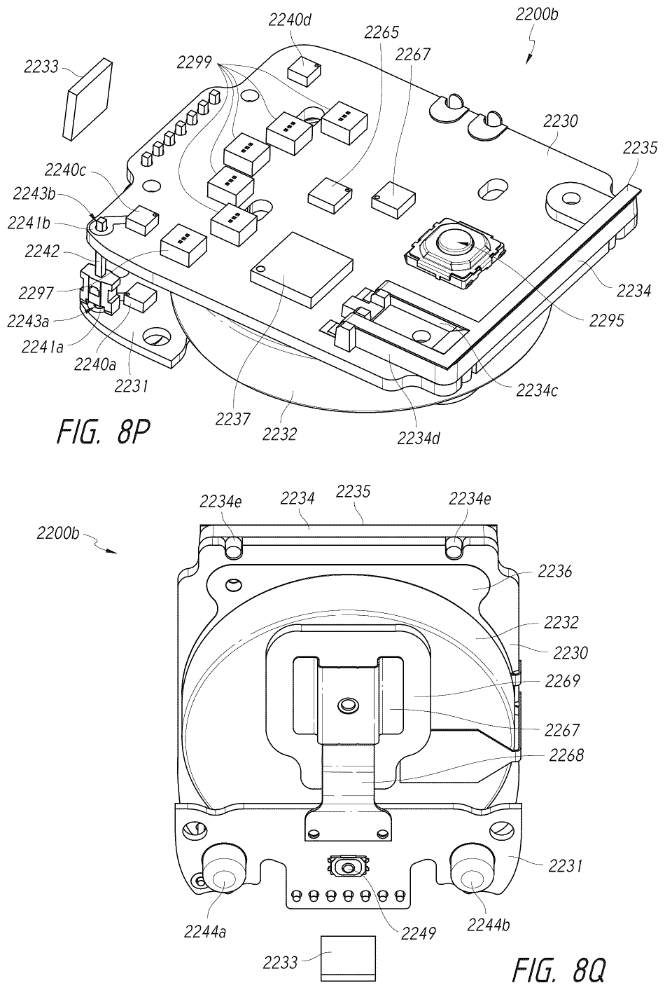

FIGS. 6A-8AE illustrate a wearable device 2000 that is a variant of the wearable device 1000 described and illustrated with respect to FIGS. 1A-5AA. FIG. 6A illustrates a top perspective view of a wearable device 2000. The wearable device 2000 can be similar to or the same as the wearable device 1000 in some or many respects. Aspects of wearable device 2000 that can be the same, similar, or have the same or similar functionality are labeled beginning with a “2” instead of a “1” as in wearable device 1000. For example, the wearable device 2000 can include a dock 2100 that is identical to the dock 1100. Furthermore, the wearable device 2000 can include a hub 2200 that is similar to the hub 1200 in many respects. For example, the wearable device 2000 can include any or all features and/or functionality as indicated in FIG. 2 and described with respect to wearable device 1000. Furthermore, the wearable device 2000 can include a housing formed by first portion 2200a and second portion 2200c, a button 2260, a display 2262, a status indicator 2215, probes 2244a, 2244b, circuit boards 2130, 2131, processor 2237, antenna 2235, frame 2234, temperature sensors 2240a, 2240b, 2240c, 2240d, motion sensor 2265, microphone 2266, microphone 2267, diaphragm 2264, and other features that are the same or similar and/or have the same or similar functionality of the housing formed by shells 1200a and 1200c, the button 1260, the display 1262, the status indicator 1215, the probes 1244a, 1244b, the circuit boards 1130, 1131, the processor 1237, the antenna 1235, the frame 1234, the temperature sensors 1240a, 1240b, 1240c, 1240d, the motion sensor 1265, the microphone 1266, the microphone 1267, and the diaphragm 1264 of wearable device 1000.

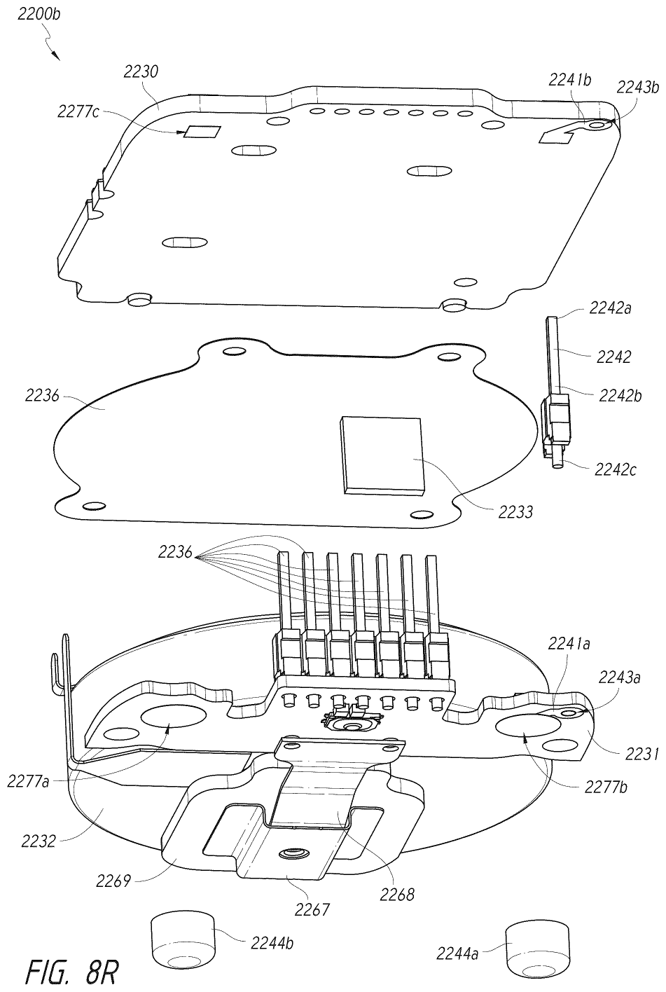

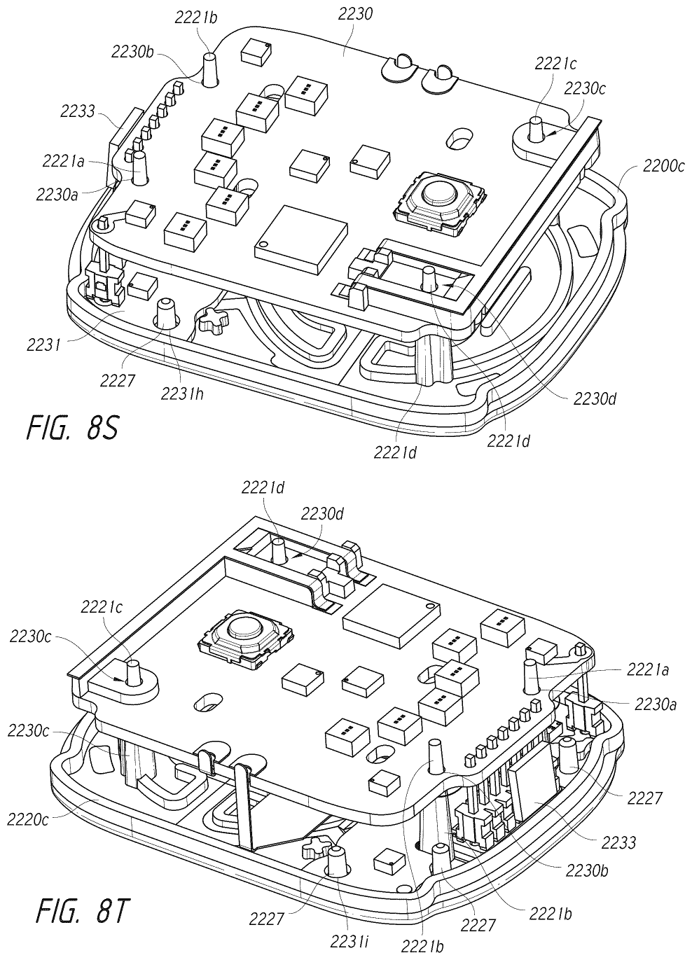

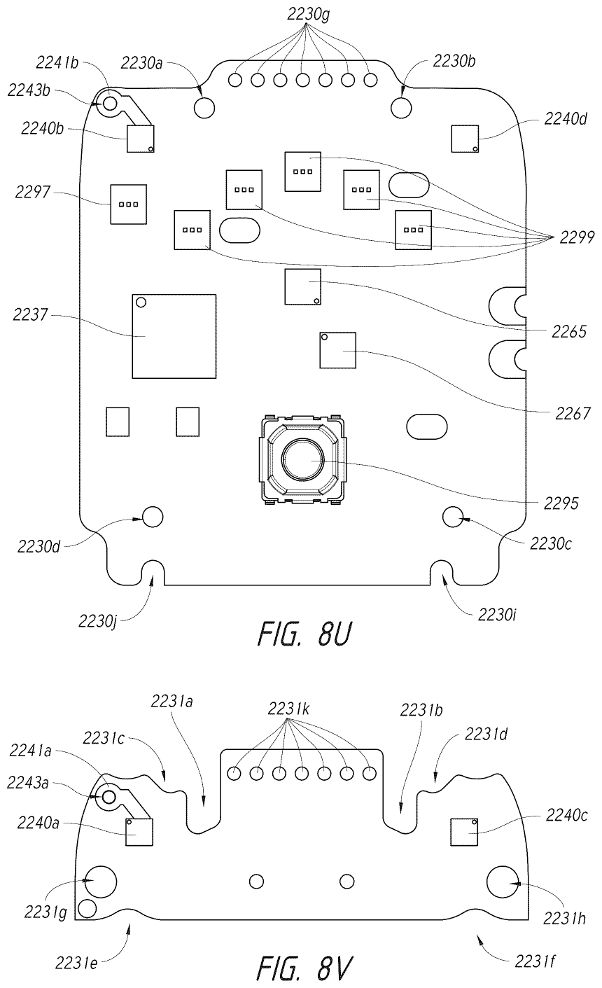

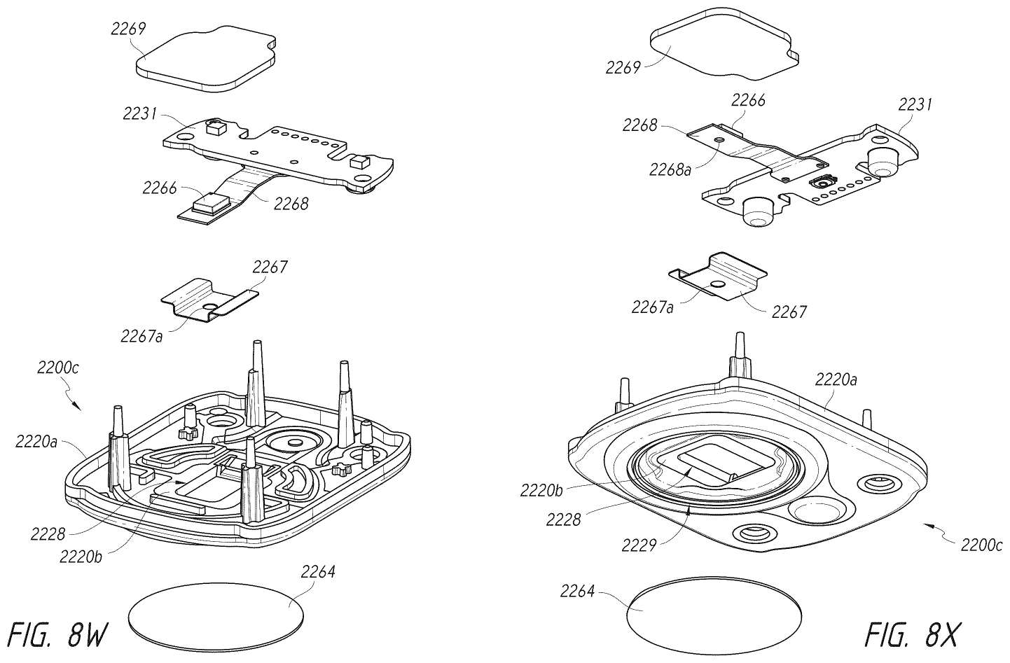

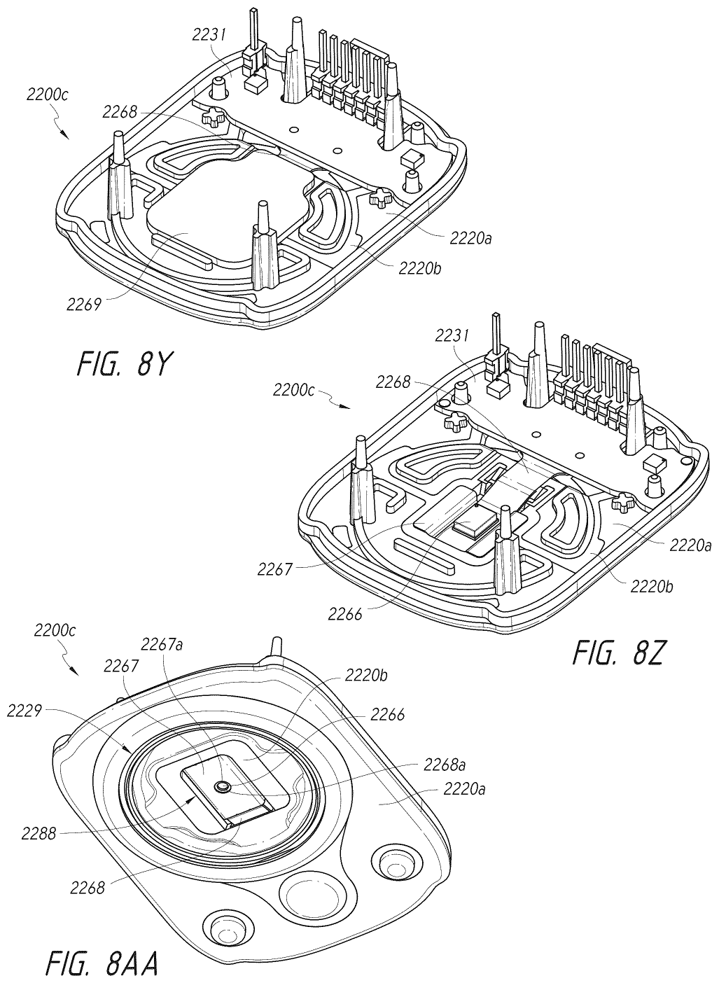

FIGS. 6B-6G illustrate top, bottom, side, and end views, respectively, of the wearable device 2000. FIG. 7A illustrates a top perspective view of the hub 2200 and the dock 2100 of the wearable device 2000 separated from one another. FIGS. 8A-8B illustrate top perspective views of the hub 2200. FIGS. 8C-8D illustrate top and bottom views, respectively, of the hub 2200. FIG. 8E illustrates a bottom perspective view of the hub 2200. FIG. 8F illustrates an end view of the hub 2200. FIGS. 8G-8H illustrate exploded views of the hub 2200. FIGS. 8I-8J illustrate bottom perspective views of a first portion 2200a of a housing of the hub 2200. FIGS. 8K-8M illustrate top and bottom perspective views of a second portion 2200c of the housing of the hub 2200. FIGS. 8N-8P illustrate top perspective views of, FIG. 8Q illustrates a bottom perspective view of, and FIG. 8R illustrates a bottom perspective partially exploded view of a third portion 2200b of the hub 2200. FIGS. 8S-8T illustrate top perspective views of portions of the hub 2200. FIG. 8U illustrates a top view of a circuit board 2230 of the hub 2200. FIG. 8V illustrates a top view of circuit board 2231 of the hub 2200. FIGS. 8W-8X illustrate top and bottom perspective partially exploded views, respectively, of a portion of the hub 2200. FIGS. 8Y-8AA illustrate top and bottom perspective views of a portion of the hub 2200. FIG. 8AB illustrates a cross-sectional view through the hub 2200 as identified in FIGS. 8C-8D. FIG. 8AC illustrates a cross-sectional view through the hub 2200 as identified in FIGS. 8C-8D. FIG. 8AD illustrates a cross-sectional view through the wearable device 2000 as identified in FIG. 6C secured to a user's skin. Dimensions W5, W6, D5, and D6 of wearable device 2000 can be similar to or the same as dimensions W1, W2, D1, and D2 of wearable device 1000 described with respect to FIG. 5Y. FIG. 8AE illustrates a cross-sectional view through the wearable device 2000 as identified in FIG. 6C secured to a user's skin. Dimensions W7, W8, D7, and D8 of wearable device 2000 can be similar to or the same as dimensions W3, W4, D3, and D4 of wearable device 1000 described with respect to FIG. 5Z.

While wearable device 2000 can be the same or similar to wearable device 1000 in some or many respects, wearable device 2000 can differ in the configuration of at least its circuit boards 2130, 2131, thermally conductive element 2242, the thermal connectivity between its temperature sensors 2240a, 2240b, 2240c, 2240d, and/or aspects of its digital stethoscope (when included).

For example, and as shown in at least FIGS. 8Q-8R, 8W-8AA and 8AB-8AC, the digital stethoscope of wearable device 2000 can include diaphragm 2264, microphone 2266, a bracket 2267, a flexible circuit 2268, a substrate 2269, and interior portion 2275 formed by the diaphragm 2264 and at least a portion of shell 2200c. The microphone 2266 can operably connect to circuit board 2231 via the flexible circuit 2268. The flexible circuit 2268 can be configured to dampen vibrations (e.g., vibrations from the housing of wearable device 2000) that can affect function of the digital stethoscope. An end of the flexible circuit 2268 that the microphone 2266 is mounted to can comprise an opening 2268a to allow vibrations/sound waves produced by diaphragm 2264 to reach the microphone 2266. This same end of the flexible circuit 2268 can rest upon bracket 2267, which can include an opening 2267a aligned with opening 2268a and for the same purpose of allowing vibrations/sound waves produced by diaphragm 2264 to reach the microphone 2266. The bracket 2267 can itself rest upon portion 2220b of shell 2200c, which can act to isolate vibrations of the housing due to its material properties (e.g., same or similar to those described with respect to portion 1220b herein). Substrate 2269 can be positioned against the microphone 2266 and act to further isolate vibrations of the housing due to its material properties (e.g. substrate 2269 can comprise a foam and/or soft material). Thus, the digital stethoscope of wearable device 2000 can be configured to acoustically isolate the microphone 2266 from vibrations and/or sound waves of the housing of wearable device 2000 and/or ambient noise. In some implementations and as shown, microphone 2266 can be substantially aligned with an axis extending through a center of the diaphragm. In some implementations, the microphone 2266 can be mounted below the flexible circuit 2268 and the bracket 2267 can be positioned above the flexible circuit 2268 (not shown). Similar to the diaphragm 1264, the diaphragm 2264 can be hermetically sealed to second portion 2200c of the housing of the wearable device 2000. Furthermore, the interior portion 2275 can be substantially sealed.

With a difference in digital stethoscope configuration between wearable device 2000 and wearable device 1000, the configuration of circuit boards 2130, 2131 with respect to battery 2232 can also be different between the wearable devices. For example and as shown in FIG. 8R, wearable device 2000 can include an electrically isolating substrate 2236 that can be positioned between the battery 2232 and the circuit board 2230.

The configuration of circuit boards 2130, 2131, thermally conductive element 2242, and temperature sensors 2240a, 2240b, 2240c, 2240d is another example in which wearable device 2000 can differ from wearable device 1000. As shown in at least FIGS. 8N-8P and 8R-8V, thermally conductive element 2242 can be at least partially positioned between and thermally connect temperature sensors 2240a and 2240c (e.g., a configuration opposite that of wearable device 1000). In such configuration, an air gap can thermally isolate temperature sensors 2240b and 2240d (e.g., a configuration opposite that of wearable device 1000). Furthermore, thermally conductive element 2242 can differ from thermally conductive element 1242. As shown in the above-mentioned figures, thermally conductive element can have an end 2242a that can extend at least partially through a hole 2243b of circuit board 2230, an end 2242c that can extend at least partially through a hole 2243a of circuit board 2231, and a portion 2242b extending between its ends 2242a, 2242c. As an example, thermally conductive element 2242 can comprise a pin header. Furthermore, thermally conductive element can comprise a metallic material, such as copper, that is thermally conductive. End 2242c of thermally conductive element 2242 can thermally connect to thermally conductive probe 2244a and temperature sensor 2240a via thermally conductive material 2241a. End 2242a of thermally conductive element 2242 can thermally connect to temperature sensor 2240c via thermally conductive material 2241b. Thermally conductive material 2241a, 2241b can comprise, for example, a copper flood within layer(s) of circuit boards 2231, 2230, respectively.

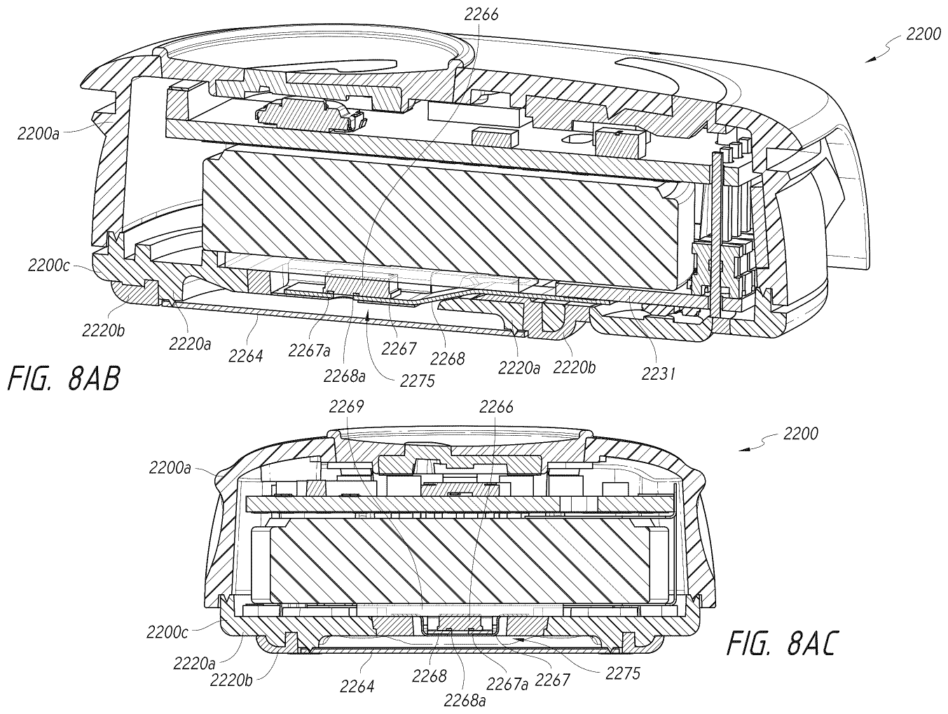

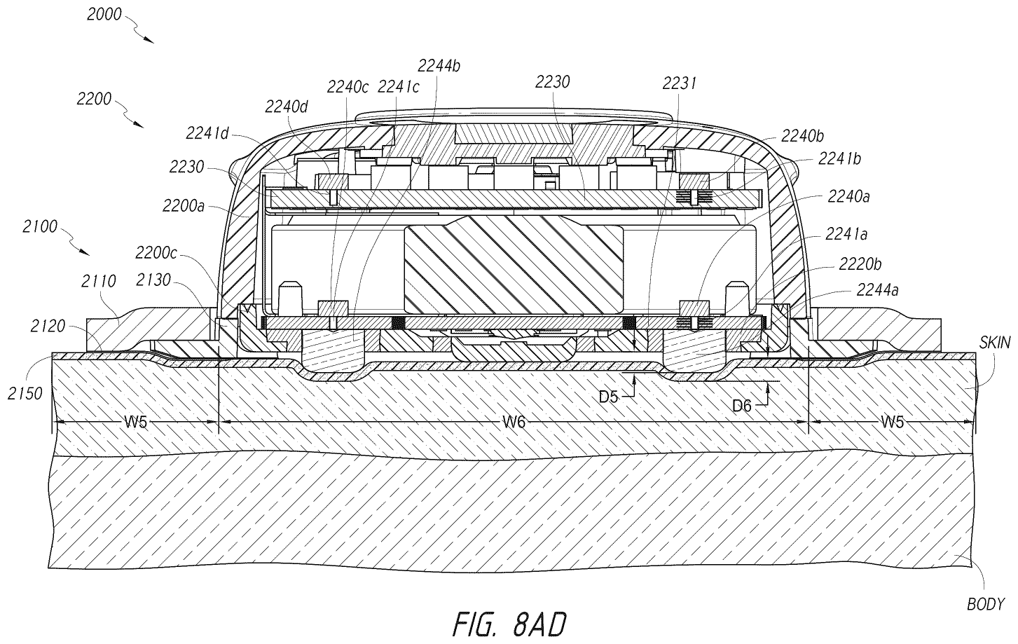

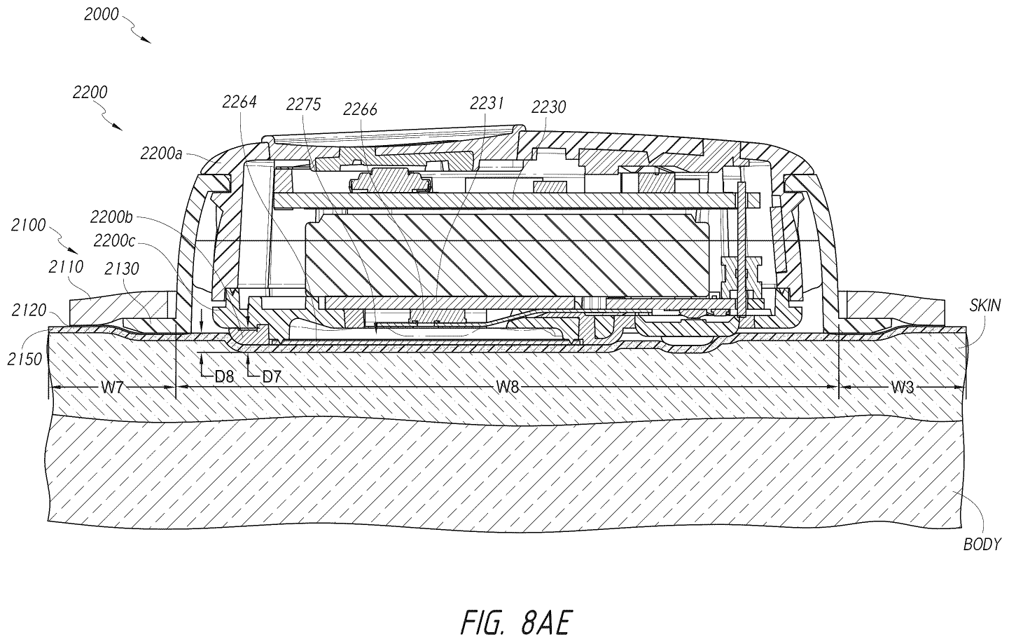

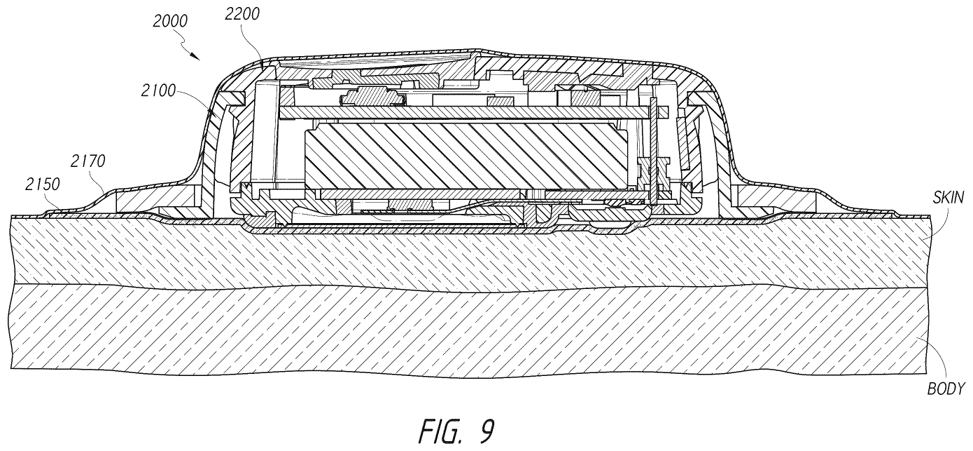

FIG. 9 shows an implementation of the wearable device 2000 attached to a user (e.g., to the skin of the user). Although wearable device 2000 is shown, this same technique can be applied to any of the wearable devices described herein (including wearable device 1000). As shown, a substrate 2170 can wrap over at least a portion of the wearable device 2000 to secure wearable device 2000 to the user. Substrate 2170 can comprise a stretchy or a non-stretchy material. Furthermore, substrate 2170 can comprise a transparent or a translucent material, for example, to allow a user and/or care provider to see through the substrate 2170. In some implementations, substrate 2170 wraps over an entirety of wearable device 2000. In some embodiments, substrate 2170 is sized and/or shaped such that it extends past the width and/or length of wearable device 2000 so as to secure directly to the user's skin along a perimeter around the wearable device 2000.

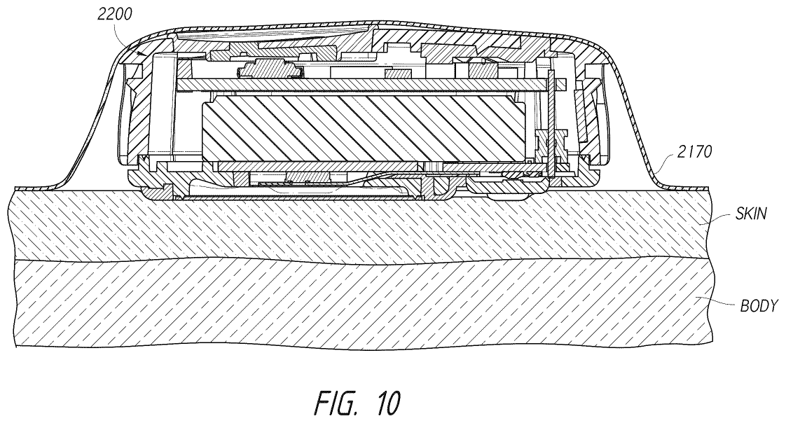

FIG. 10 shows an implementation of a portion of the wearable device 2000 attached to a user (e.g., to the skin of the user). Although wearable device 2000 is shown, this same technique can be applied to any of the wearable devices described herein (including wearable device 1000). As shown, the substrate 2170 described with respect to FIG. 9 can wrap over a hub 2200 of the wearable device 2000 to secure the hub 2200 to the user. In other words, in some implementations the hub 2200 can be secured to the user without the dock 2100. In some implementations, substrate 2170 wraps over an entirety of hub 2200. In some embodiments, substrate 2170 is sized and/or shaped such that it extends past the width and/or length of hub 2200 so as to secure directly to the user's skin along a perimeter around the hub 2200.

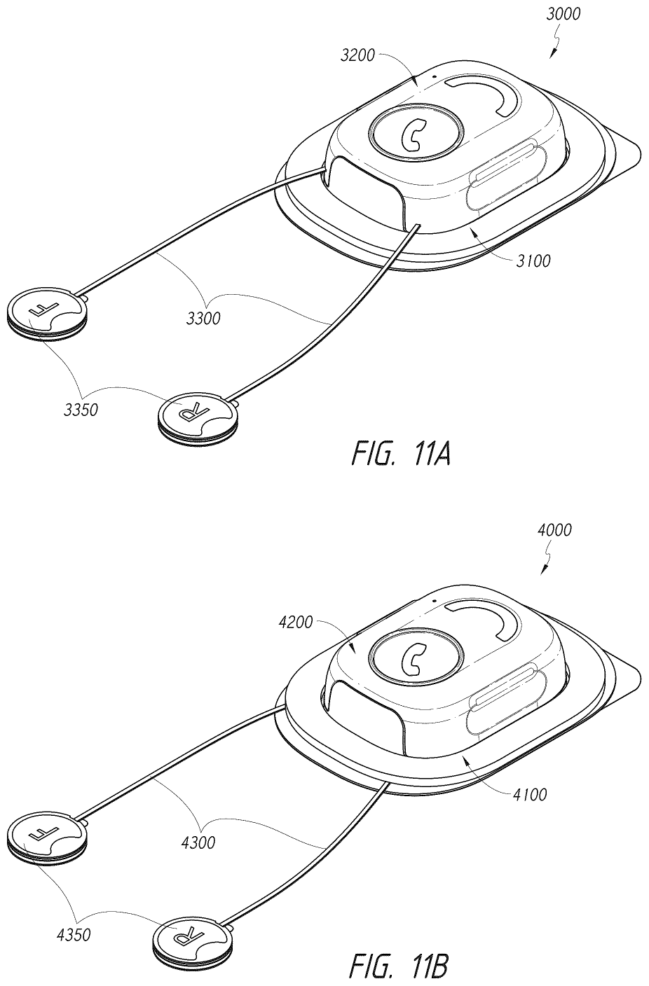

FIGS. 11A-11B illustrate a wearable device 3000, 4000 that are variants of the wearable devices 1000, 2000 described and illustrated with respect to FIGS. 1A-5AA and FIGS. 6A-8AE. The wearable devices 3000, 4000 can be identical to the wearable devices 1000, 2000 (and as such are labeled beginning with a “3” and a “4” instead of a “1” and a “2”) except that they can include ECG functionality. For example and as shown, the wearable devices 3000, 4000 can include cables 3300, 4300 and corresponding external ECG electrodes 3350, 4350. Such ECG electrodes 3350, 4350 can be configured to attach to the user and output one or more signals responsive to the user's cardiac electrical activity. The one or more hardware processors of wearable devices 3000, 4000 can be configured to receive the one or more signals from the external ECG electrodes (e.g., via their respective cables 3300, 4300) responsive to the user's cardiac electrical activity and determine an ECG of the user responsive to said one or more signals. Wearable device 3000 can differ from wearable device 4000 in that the cables 3300 and corresponding external ECG electrodes 3350 can extend from a hub 3200 of the wearable device 3000. In such configuration, the dock 3100 of wearable device 3000 does not comprise any electronic components. Alternatively, wearable device 4000 can be configured such that the cables 4300 and corresponding external ECG electrodes 4350 connect with corresponding electrical connectors (not shown) of the dock 4100, which can in turn electrically connect with corresponding electrical connectors (not shown) of the hub 4200 when the dock 4100 and hub 4200 are connected to one another. In some implementations, wearable devices 3000, 4000 can include one or more internal ECG electrodes configured to output one or more signals responsive to the user's cardiac electrical activity. Such internal ECG electrodes can be integrated into the hubs 3200, 4200 or the docks 3100, 4100. In such implementations, the one or more hardware processors of wearable devices 3000, 4000 can be configured to receive the one or more signals from the internal ECG electrodes responsive to the user's cardiac electrical activity and determine an ECG of the user responsive to the one or more signals. Wearable devices 3000, 4000 can include ECG functionality similar or identical to and/or incorporate any of the features described with respect to any of the devices, assemblies, methods, and/or systems described and/or illustrated in U.S. Pat. Pub. No. US2020/0329993, filed Apr. 16, 2020, titled “ELECTROCARDIOGRAM DEVICE,” in U.S. Pat. Pub. No. US2022/0233128, filed Apr. 4, 2022, titled “ELECTROCARDIOGRAM DEVICE,” and in U.S. Pat. App. No. 63/486,456, filed Feb. 2, 2023, titled “ELECTROCARDIOGRAM DEVICE” incorporated by reference herein.

FIG. 12 illustrates two call devices 5100, 6100 (which can also be referred to herein as a “device”, a “care provider call device”, a “nurse call device”, or a “caregiver call device”) in an example medical environment (which may also be referred to as a “healthcare environment” or “care environment”) in which a user 1 is positioned in a hospital bed 3. Although two nurse call devices 5100, 6100 are illustrated in FIG. 12, this is merely to illustrate two example locations where devices 5100, 6100 can be located in relation to a patient 1 in a medical environment. As shown, a call device 5100, 6100 can be configured to secure to a surface of the hospital bed 3. As also shown, a call device 5100, 6100 can be configured to secure to a surface near or adjacent the hospital bed 3, such as a surface of a bedside desk or drawer. Advantageously, the call device 5100, 6100 can be configured to secure to a surface that is within reach of and/or convenient for the user 1, which can be customized to the user 1 and account for any limitations in mobility of the user 1. FIG. 12 also illustrates an example patient monitor 10 that can be mounted to a wall, for example, via a wall mount as shown. Also shown is a wearable patient monitoring device 20 (which may also be referred to as a “wearable device” or a “physiological monitoring device”) and an oximetry sensor 30. Patient monitoring device 20 can be similar to any of the monitors described and/or illustrated in U.S. Pat. No. 10,149,616, filed Feb. 7, 2013, titled “WIRELESS PATIENT MONITORING DEVICE,” incorporated by reference herein in its entirety, and oximetry sensor 30 can be similar or identical to any of the optical sensors described and/or illustrated in the same. Although only patient monitoring device 20 and oximetry sensor 30 are illustrated attached to user 1 in the example medical environment of FIG. 12, various alternative or additional patient monitors and/or sensors can be utilized alongside call device 5100, 6100 in the example medical environment and can be attached to user 1. For example, any of the wearable devices described herein (e.g., wearable devices 1000, 2000, 3000, and/or 4000) can be secured to the user 1.

Also illustrated in FIG. 12 is a patient monitor 10. Patient monitor 10 can be configured to display one or more or a plurality of physiological parameters of the user 1 that are received (for example, wirelessly received) by one or more physiological sensors (such as from monitoring device 20, oximetry sensor 30, or any of the wearable devices 1000, 2000, 3000, and/or 4000 described herein). Patient monitor 10 can be, and/or can be configured to operate in a manner, similar or identical to any of the medical monitoring hubs described and/or illustrated in U.S. Pat. No. 10,010,276, filed Oct. 6, 2014, titled “REGIONAL OXIMETRY USER INTERFACE,” and incorporated herein in its entirety.

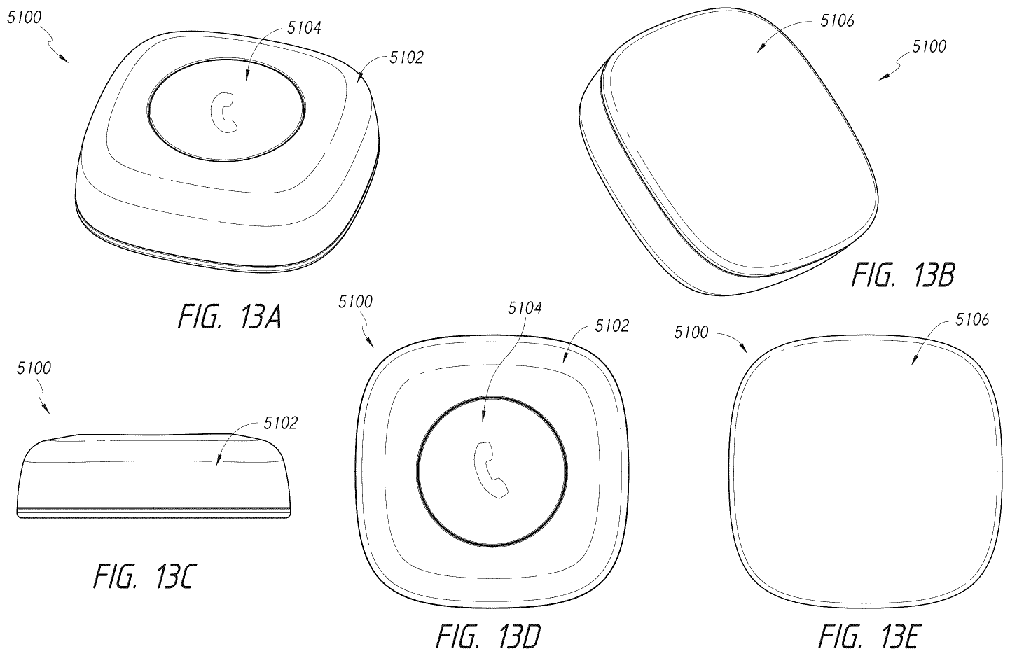

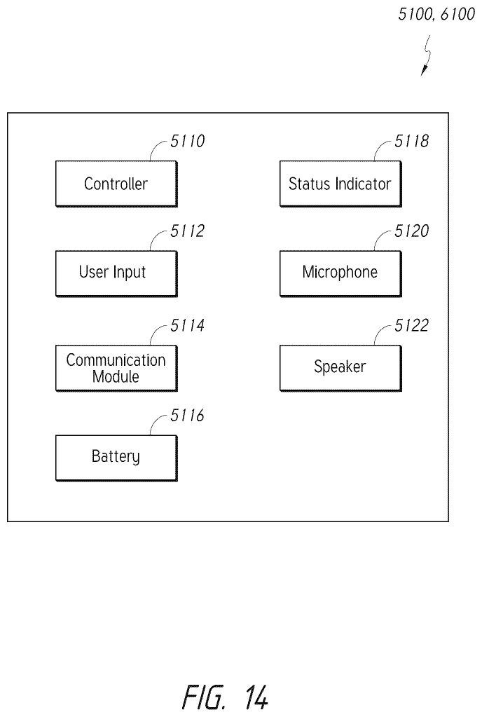

FIGS. 13A-13E illustrate views of the call device 5100. FIGS. 15A-15E illustrates views of another call device 6100. FIG. 14 illustrates a schematic diagram of certain features that can be included in call device 5100, 6100. Although the phrase “nurse call device” can be used herein, such language is not intended to be limiting nor is it intended to mean that any of the devices disclosed herein must be utilized to communicate with a “nurse”. Any of the devices described herein (such as device 5100, 6100) can be utilized to communicate with any person (for example, via a device associated with such person) who may provide care to a user (for example, a patient) and/or who may be tasked with monitoring the user's wellbeing.

With reference to FIGS. 13A-13E and 15A-15E, nurse call device 5100, 6100 can include a main body 5102, 6102. Main body 5102, 6102 can be of a variety of shapes and sizes. In some implementations, main body 5102, 6102 has at least one flat side and/or surface that allows device 5100, 6100 to rest flat and/or flush upon a surface, for example, of a hospital bed (or a portion of a hospital bed such as a wall or rail of a hospital bed), a desk, a drawer, and/or a table, among other surfaces. In some implementations, device 5100, 6100 includes an adhesive material on a portion thereof (such as a portion of the main body 5102, 6102) that allows device 5100, 6100 to secure in a variety of orientations relative to a surface. Such adhesive material can allow the device 5100, 6100 to secure, for example, to a portion of hospital bed 3, such as a siderail of hospital bed 3, and/or a desk as shown in FIG. 12. In some implementations, device 5100, 6100 includes an adhesive material on a bottom and/or flat surface of main body 5102, 6102 that enables the device 5100, 6100 to secure to a surface, for example, in a substantially vertical orientation relative to a siderail of the hospital bed 3 and/or in a substantially horizontal orientation as shown in FIG. 12 (although other orientations, such as between vertical and horizontal, are possible as well). For example, in some implementations, device 5100, 6100 includes an adhesive material along a bottom and/or flat surface of main body 5102, 6102, as indicated by numerals 5106 and 6106 in the figures. In some implementations, device 5100, 6100 is configured to removably secure (e.g., via adhesive material) to a surface. As discussed in more detail below and shown in, device 5100, 6100 can include a user input 5112 that allows a user to interact with the device 5100, 6100, for example, to cause the devices 5100, 6100 to carry out one or more actions (such as wirelessly communicating with a separate device associated with a care provider). Such user input 5112 can be disposed in a position relative to the main body 5102, 6102 such that it is accessible to a user when the device 5100, 6100 is secured to a surface. FIGS. 13A, 13D and 15A, 15D illustrate a button 5104, 6104 which can be an implementation of such user input 5112. Aspects of user input 5112 and button 5104, 6104 are described in further detail below.

With reference to FIG. 14, device 5100, 6100 can include a controller 5110, a user input 5112, a communication module 5114, and a battery 5116. Controller 5110 can be configured to control operation of device 5100, 6100. Controller 5110 can include a hardware processor and a storage device, and such storage device can be coupled with the hardware processor. In some implementations, controller 5110 is embodied in a printed circuit board. Controller 5110 (for example, utilizing such hardware processor) can be configured among other things, to receive and/or process data, execute instructions to perform one or more functions, and/or control the operation of the device 5100, 6100. For example, controller 5110 can be configured to control operation of communication module 5114 based on input received from the user input 5112. Such storage device can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like.

User input 5112 can allow a user to interact with device 5100, 6100, for example, to cause the device 5100, 6100 to carry out one or more actions. For example, user input 5112 can allow device 5100, 6100 to receive input from a user (for example, a patient) to communicate with a separate device associated with a care provider. Such communication can be associated with a request for the care provider to visit the user, for example, to attend to a need. In the illustrated implementations of device 5100, 6100 in at least FIGS. 13A and 15A, each comprise a button 5104, 6104 that can be pressed by a user, and such button 5104, 6104 is an example implementation of user input 5112.

Button 5104, 6104 can be operably positioned relative to main body 5102, 6102 and, in some implementations, movable relative to main body 5102, 6102. Button 5104, 6104 can include an actuator and a switch. In some implementations, the actuator is configured to move between a first position in which the actuator is not in contact with the switch to a second position in which the actuator is in contact with the switch. In some of such implementations, the actuator is biased toward such first position. In some implementations, the actuator is configured to move vertically (for example, relative main body 5102, 6102) when moved from and/or between the first to the second positions. In some implementations, the actuator is configured to move laterally (for example, relative main body 5102, 6102) when moved from and/or between the first to the second positions, for example, according to a sliding type configuration. In some of such implementations, the switch is configured to communicate with controller 5110 when the actuator is in the first position, or alternatively, when the actuator is in the second position. Such communication can be, for example, via one or more signals transmitted (for example, via circuitry of device 5100, 6100) to controller 5110. Such signal(s) may be referred to as “user input signals”. In some implementations, controller 5110 is configured to: determine an amount of time that the actuator is in the second position; and instruct communication module 5114 to wirelessly transmit one or more communication signals to a separate device associated with a care provider when said amount of time is greater than or equal to a first threshold. In some implementations, controller 5110 is configured to instruct communication module 5114 to wirelessly transmit one or more communication signals to a separate device associated with a care provider only when said amount of time is greater than or equal to the first threshold. For example, in some implementations, controller 5110 is configured to instruct communication module 5114 to wirelessly transmit one or more communication signals to a separate device associated with a care provider only when an actuator of button 5104, 6104 has been held in the above-described second position for at least 1 second, at least 2 seconds, at least 3 seconds, at least 4 seconds, or at least 5 seconds. Such implementations can advantageously prevent inadvertent/accidental operation of the device 5100, 6100 by a user in some cases, for example, where a portion of the user's body inadvertently pressed against the button 5104, 6104.

User input 5112 can be implemented as an alternative mechanism than button 5104, 6104. In some implementations, user input 5112 does not include an actuator and/or a switch, and/or user input 5112 does not include a component that is movable. In some implementations, user input 5112 comprises a capacitive or resistive sensor that can be utilized to receive input from a user.

In some implementations, user input 5112 (e.g., implemented as button 5104, 6104) can be recessed relative to the main body 5102, 6102 and/or otherwise positioned below a surface (e.g., a surface opposite surface 5106, 6106) of the main body 5102, 6102. Such positioning can help prevent a user from inadvertently pressing and/or activating user input 5112.

Communication module 5114 can facilitate communication (for example, wireless communication) between device 5100, 6100 and separate devices, such as separate monitoring and/or mobile devices associated with one or more care providers. Communication module 5114 can be configured to allow device 5100, 6100 to wirelessly communicate with other devices, systems, and/or networks over any of a variety of communication protocols including, for example, Wi-Fi (802.11x), Bluetooth®, ZigBee®, Z-wave®, cellular telephony, infrared, near-field communications (NFC), RFID, satellite transmission, proprietary protocols, combinations of the same, and the like. Communication module 5114 can be and/or include a wireless transceiver. Communication module 5114 can transmit one or more signals (which may be referred to as “communication signals”) to a separate device associated with a care provider responsive to being instructed to do so by controller 5110 (for example, based on input received by user input 5112).

Device 5100, 6100 can include a battery 5116. Battery 5116 can be rechargeable (for example, via inductive or wireless charging) or non-rechargeable. Battery 5116 can provide power for the hardware components of the device 5100, 6100 described herein (such as the controller 5110, user input 5112, communication module 5114, and/or other components of device 5100, 6100). Battery 5116 can be, for example, a lithium battery. In some implementations, device 5100, 6100 includes a status indicator 5118 configured to indicate a status of device 5100, 6100, such as whether device 5100, 6100 is in an operational (“on”) mode, whether device 5100, 6100 is pairing or has paired with a separate device, whether an error has been detected, and/or a power level of device 5100, 6100. For example, device 5100, 6100 can include an emitter configured to emit light of one or more wavelengths to indicate a status of device 5100, 6100. Such emitter can include one or more light-emitting diodes (LEDs) and can emit light of certain colors to indicate certain statuses of device 5100, 6100. For example, such emitter can emit a green light to indicate that device 5100, 6100 is powered “on” or a red light to indicate device 5100, 6100 is “off”. In some implementations, the emitter blinks lights (for example, of a certain color) when an error has been detected and/or a power level of device 5100, 6100 is below a threshold. In some implementations, device 5100, 6100 includes an opening in a portion thereof, for example, in button 5104, 6104 where device 5100, 6100 includes such button 5104, 6104, and/or in a portion of main body 5102, 6102. Such opening can allow light emitted from the emitter to be visible from a location outside an interior of device 5100, 6100. Additionally or alternatively, device 5100, 6100 (for example, main body 5102, 6102) can comprise a transparent or semi-transparent material that allows light emitted from the emitter to be seen from a location outside an interior of device 5100, 6100. In some implementations, device 5100, 6100 does not comprise: any sensors for measuring and/or monitoring physiological parameters; any sensors for measuring and/or monitoring motion, location, and/or position of a user; a display screen; and/or any cables. Such implementations can advantageously minimize power consumption of battery 5116 and/or otherwise simplify use of device 5100, 6100 for a user.

In some implementations, device 5100, 6100 includes a microphone 5120. Microphone 5120 can be utilized by device 5100, 6100 for a variety of purposes. In some implementations, controller 5110 is configured to instruct communication module 5114 to transmit one or more communication signals based on sound detected by microphone 5120. For example, controller 5110 can be configured to instruction communication module 5114 to transmit one or more communication signals to a separate device (for example, associated with a care provider) when a volume of the detected sound is above a certain threshold (which may be indicative of an urgent need from a patient). Such communication signal(s) transmitted by communication module 5114 to the separate device can correspond to an audio and/or visual alert that can be played/displayed on the separate device and/or can be raw and/or processed signals based on the sound detected by the microphone 5120. In some implementations, device 5100, 6100 includes a speaker 5122 configured to output sound. Such outputted sound can be based on audio signals received by communication module 5114, for example, from a separate device associated with a care provider. In some implementations, device 5100, 6100 can act as an audio intercom between a user and a care provider. In some of such implementations, microphone 5120 is configured to receive audio from the user when the actuator as described above is in the second position (or alternatively, the first position), for example, for a time period that is greater than or equal to a threshold (which can be the same as the first threshold described above). Furthermore, in some implementations, controller 5110 is configured to instruct communication module 5114 to transmit such received audio to the separate device of the caregiver upon a change in the position of the actuator (e.g., from the second position to the first position or vice versa).

Device 5100, 6100 can be configured to communicate with a variety of separate devices. For example, device 5100, 6100 can be configured to communicate with one or more physiological sensors on a user 1, such as patient monitoring device 20 and/or oximetry sensor 30 illustrated in FIG. 12 (among others) and/or with one or more patient monitors, such as patient monitor 10, also illustrated in FIG. 12. In some cases, device 5100, 6100 is configured to communicate with a separate device associated with a care provider indirectly by sending one or more communication signals to patient monitor 10. For example, upon receiving input at user input 5112 associated with a request to visit the user 1, controller 5110 can instruct communication module 5114 to send one or more signals to patient monitor 10 that instruct the patient monitor 10 to communicate with the separate device of the care provider. Such configurations may be advantageous when a wireless communication range of device 5100, 6100 is less than that required to reach the separate device. In some implementations, device 5100, 6100 is configured to wirelessly communicate over a first range and the patient monitor 10 is configured to wirelessly communication over a second range that is greater than the first range. In some implementations, upon receiving input at user input 5112 associated with a request to visit the user 1, patient monitor 10 can display a visual indication and/or alert and/or an audible indication and/or alert to visibly and/or audibly notify nearby care providers or personnel that the user 1 requests a visit.

### Additional Implementations

1. A wearable device for monitoring and displaying orientation of a user, the wearable device comprising:
a housing configured to be secured to a portion of the user's body;
an accelerometer and a gyroscope, each of the accelerometer and the gyroscope positioned within an interior of the housing;
a display positioned along an exterior portion of the housing; and
one or more hardware processors positioned within the interior of the housing;
wherein the one or more hardware processors are configured to:
   receive one or more signals generated by the accelerometer responsive to linear acceleration of the user;
   receive one or more signals generated by the gyroscope responsive to angular velocity of the user;
   determine orientation of the user relative to a surface over time based on said received one or more signals generated by each of the accelerometer and the gyroscope; and
   change an appearance of the display based on said determined orientation over time to provide a care provider with information for assessing a risk of pressure ulcer formation.

2. The wearable device of Implementation 1, wherein the one or more hardware processors are further configured to:
for each respective orientation of a plurality of orientations of the user with respect to the surface:
   increase a value of a timer associated with the respective orientation when the user is in the respective orientation; and
   decrease the value of the timer when the user is not in the respective orientation; and
for each respective one of a plurality of portions of the display:
   change an appearance of the respective one of the plurality of portions based on the value of the timer associated with one of said plurality of orientations.

3. The wearable device of Implementation 2, wherein:
a first one of said plurality of orientations is associated with a left side orientation of the user with respect to the surface;
a second one of said plurality of orientations is associated with a right side orientation of the user with respect to the surface; and
a third one of said plurality of orientations is associated with a supine orientation of the user with respect to the surface.

4. The wearable device of Implementation 2 or 3, wherein, for each respective one of the plurality of portions of the display, the one or more hardware processors are further configured to:
cause the appearance of the respective one of the plurality of portions to have a first color when the value of the timer is greater than or equal to a threshold; and
cause the appearance of the respective one of the plurality of portions to have a second color when the value of the timer is below the threshold, said second color being different than said first color.

5. The wearable device of Implementation 4, wherein:
said display comprises a border having at least a first edge and a second edge; and
each of said plurality of portions of the display comprises a line extending between the first and second edges of the border.

6. The wearable device of any of Implementations 1-5, wherein:
said display only illustrates the user's orientation and does not include any other information; and/or
said display is only utilized to graphically illustrate information relating to the user's orientation over time.

7. The wearable device of any of Implementation 1-6, wherein:
the wearable device comprises a first portion and a second portion, the first portion comprising a frame and a substrate coupled to the frame, the second portion comprising said housing of said wearable device;
the frame and the housing are removably secured to one another; and
the substrate is configured to secure to skin of the user.

8. The wearable device of any of Implementations 1-7, further comprising:
a first temperature sensor and a second temperature sensor, each of said first and second temperature sensors positioned within the interior of the housing and configured to generate one or more signals responsive to detected thermal energy, said first temperature sensor operably positioned to be closer to the user's skin than the second temperature sensor when the housing is secured to the portion of the user's body;
wherein the one or more hardware processors are further configured to:
   receive said one or more signals generated by each of said first and second temperature sensors; and
   determine one or more body temperature values of the user based on at least said received one or more signals generated by each of said first and second temperature sensors.

9. The wearable device of Implementation 8, further comprising:
a third temperature sensor and a fourth temperature sensor, each of said third and fourth temperature sensors positioned within the interior of the housing and configured to generate one or more signals responsive to detected thermal energy, said third temperature sensor operably positioned to be closer to the user's skin than the fourth temperature sensor when the housing is secured to the portion of the user's body;
wherein the one or more hardware processors are further configured to:
   receive said one or more signals generated by each of said third and fourth temperature sensors; and
   determine said one or more body temperature values of the user based on said received one or more signals generated by each of said first, second, third, and fourth temperature sensors.

10. The wearable device of Implementation 9, further comprising a thermally conductive element positioned at least partially between the third and fourth temperature sensors

11. The wearable device of Implementation 10, wherein said thermally conductive element comprises a metal strip.

12. The wearable device of Implementation 11, wherein said metal strip is at least partially bent.

13. The wearable device of any of Implementations 8-12, wherein the first and second temperature sensors are thermally insulated from one another by an air gap

14. The wearable device of Implementation 13, wherein:
the wearable device further comprises a first circuit board and a second circuit board, the first and second circuit boards spaced from one another, the first circuit board positioned closer to the skin of the user than the second circuit board when the housing is secured to the portion of the user's body;
said first temperature sensor is mounted to the first circuit board and the second temperature sensor is mounted on the second circuit board;
said third temperature sensor is mounted to the first circuit board and spaced from the first temperature sensor;
said fourth temperature sensor is mounted to the second circuit board and spaced from the second temperature sensor;
a distance between the first temperature sensor and the second circuit board at least partially defines said air gap; and
said thermally conductive element is positioned between the third temperature sensor and a portion of the second circuit board that is adjacent to the fourth temperature sensor.

15. The wearable device of any of Implementations 8-14, wherein said first and second temperature sensors are substantially aligned with one another and wherein said third and fourth temperature sensors are substantially aligned with one another.

16. The wearable device of any of Implementation 1-15, wherein the housing is configured to be secured to the user's chest.

17. The wearable device of any of Implementations 1-16, wherein:
the housing further comprises a top portion, a bottom portion, and an opening in the bottom portion of the housing, said bottom portion configured to face toward the user's body when the housing is secured to the portion of the user's body;
the wearable device further comprises a diaphragm coupled to the bottom portion of the housing and covering said opening, wherein, when said housing is secured to the portion of the user's body, at least a portion of said diaphragm is configured to vibrate responsive to cardiac and/or lung activity of the user, said vibration of said diaphragm generating sound waves within at least a portion of the interior of the housing;
a microphone positioned within the interior of the housing, said microphone configured to detect said sound waves generated by said diaphragm and generate one or more signals based on said detected sound waves; and
one or more hardware processors positioned within the interior of the housing, said one or more hardware processors configured to receive said one or more signals generated by said microphone and determine at least one of cardiac function and lung function based upon said received one or more signals.

18. A wearable device comprising:
a housing configured to be secured to a portion of a user's body, the housing comprising an interior, a top portion, and a bottom portion, said bottom portion configured to face toward the user's body when the housing is secured to the portion of the user's body;
an opening in the bottom portion of the housing;
a diaphragm coupled to the bottom portion of the housing and covering said opening, wherein, when said housing is secured to the portion of the user's body, at least a portion of said diaphragm is configured to vibrate responsive to cardiac and/or lung activity of the user, said vibration of said diaphragm generating sound waves within at least a portion of the interior of the housing;
a microphone positioned within the interior of the housing, said microphone configured to detect said sound waves generated by said diaphragm and generate one or more signals based on said detected sound waves; and
one or more hardware processors positioned within the interior of the housing, said one or more hardware processors configured to receive said one or more signals generated by said microphone and determine at least one of cardiac function and lung function based upon said received one or more signals.

19. The wearable device of Implementation 18, further comprising a circuit board positioned within the interior of the housing, wherein said vibration of said diaphragm generates sound wave within a portion of the interior of the housing that is defined between the circuit board and the diaphragm.

20. The wearable device of Implementation 19, wherein:
the circuit board comprises a first surface, a second surface opposite the first surface, and a hole extending through the circuit board, said second surface facing toward said diaphragm;
the microphone is mounted to the first surface of the circuit board adjacent said hole; and
said hole allows the sound waves generated by said vibration of the diaphragm to pass from the portion of the interior of the housing defined between the circuit board and the diaphragm through the circuit board and to the microphone.

21. The wearable device of Implementation 20, wherein said microphone is embodied in a microphone chip.

22. The wearable device of Implementation 21, wherein said microphone chip is not aligned with an axis extending through a center of said hole.

23. The wearable device of any of Implementations 19-22, wherein the portion of the interior of the housing defined between the circuit board and the diaphragm is substantially sealed.

24. The wearable device of any of Implementations 19-23, wherein the only opening into the portion of the interior of the housing defined between the circuit board and the diaphragm is the hole in the circuit board.

25. The wearable device of any of Implementations 18-24, wherein:
the wearable device comprises a first portion and a second portion, the first portion comprising a frame and a substrate coupled to the frame, the second portion comprising said housing of said wearable device;
the frame and the housing are removably secured to one another; and
the substrate is configured to secure to skin of the user.

26. The wearable device of Implementation 25, wherein said substrate is configured to be positioned between the diaphragm and skin of the user when the wearable device is secured to the user's body.

27. The wearable device of any of Implementations 18-24, further comprising:
a first temperature sensor and a second temperature sensor, each of said first and second temperature sensors positioned within the interior of the housing and configured to generate one or more signals responsive to detected thermal energy, said first temperature sensor operably positioned to be closer to the user's skin than the second temperature sensor when the housing is secured to the portion of the user's body;
wherein the one or more hardware processors are further configured to:
   receive said one or more signals generated by each of said first and second temperature sensors; and
   determine one or more body temperature values of the user based on at least said received one or more signals generated by each of said first and second temperature sensors.

28. The wearable device of Implementation 27, further comprising:
a third temperature sensor and a fourth temperature sensor, each of said third and fourth temperature sensors positioned within the interior of the housing and configured to generate one or more signals responsive to detected thermal energy, said third temperature sensor operably positioned to be closer to the user's skin than the fourth temperature sensor when the housing is secured to the portion of the user's body;
wherein the one or more hardware processors are further configured to:
   receive said one or more signals generated by each of said third and fourth temperature sensors; and
   determine said one or more body temperature values of the user based on said received one or more signals generated by each of said first, second, third, and fourth temperature sensors.

29. The wearable device of Implementation 28, further comprising a thermally conductive element positioned at least partially between the third and fourth temperature sensors

30. The wearable device of Implementation 29, wherein said thermally conductive element comprises a metal strip.

31. The wearable device of Implementation 30, wherein said metal strip is at least partially bent.

32. The wearable device of any of Implementations 27-31, wherein the first and second temperature sensors are thermally insulated from one another by an air gap.

33. The wearable device of Implementation 32, wherein:
the wearable device further comprises a first circuit board and a second circuit board, the first and second circuit boards spaced from one another, the first circuit board positioned closer to the skin of the user than the second circuit board when the housing is secured to the portion of the user's body;
said first temperature sensor is mounted to the first circuit board and the second temperature sensor is mounted on the second circuit board;
said third temperature sensor is mounted to the first circuit board and spaced from the first temperature sensor;
said fourth temperature sensor is mounted to the second circuit board and spaced from the second temperature sensor;
a distance between the first temperature sensor and the second circuit board at least partially defines said air gap; and
said thermally conductive element is positioned between the third temperature sensor and a portion of the second circuit board that is adjacent to the fourth temperature sensor.

34. The wearable device of any of Implementations 26-33, wherein said first and second temperature sensors are substantially aligned with one another and wherein said third and fourth temperature sensors are substantially aligned with one another.

35. The wearable device of any of Implementations 18-34, further comprising:
an accelerometer and a gyroscope, each of the accelerometer and the gyroscope positioned within the interior of the housing; and
a display positioned along an exterior portion of the housing;
wherein the one or more hardware processors are further configured to:
   receive one or more signals generated by the accelerometer responsive to linear acceleration of the user;
   receive one or more signals generated by the gyroscope responsive to angular velocity of the user;
   determine orientation of the user relative to a surface over time based on said received one or more signals generated by each of the accelerometer and the gyroscope; and
   change an appearance of the display based on said determined orientation over time to provide a care provider with information for assessing a risk of pressure ulcer formation.

36. A device for wirelessly communicating with a care provider in a hospital, the device comprising:
a main body;
a user input coupled to the main body and configured to generate one or more user input signals responsive to a user interacting with the user input;
a communication module configured to allow the device to wirelessly communicate with a separate device that is associated with the care provider;
a controller in communication with the user input and the communication module, wherein the controller is configured to:
   receive said one or more user input signals generated by said user input; and
   instruct the communication module to wirelessly transmit one or more communication signals to the separate device based on said received one or more user input signals; and
an adhesive material positioned along a portion of the main body and configured to secure the device to a surface of a hospital bed.

37. The device of Implementation 36, wherein said user input comprises an actuator and a switch, and wherein, the actuator is configured to move from a first position in which the actuator does not contact the switch to a second position in which the actuator contacts the switch.

38. The device of Implementation 37, wherein the switch is configured to generate said one or more user input signals when the actuator is in the second position.

39. The device of Implementation 37 or 38, wherein the controller is configured to:
determine an amount of time that the actuator is in the second position; and
instruct the communication module to wirelessly transmit the one or more communication signals to the separate device when said amount of time is greater than or equal to a first threshold.

40. The device of Implementation 39, wherein the controller is further configured to instruct the communication module to wirelessly transmit the one or more communication signals to the separate device only when said amount of time is greater than or equal to the first threshold.

41. The device of any of Implementations 36-40, wherein said main body comprises said adhesive material.

42. The device of Implementation 41, further comprising a release liner configured to cover said adhesive material, wherein the release liner is removable from the adhesive material and the device.

43. The device of any of Implementations 36-42, further comprising a battery.

44. The device of any of Implementations 36-43, wherein the device does not comprise:
any sensors for measuring and/or monitoring physiological parameters;
any sensors for measuring and/or monitoring motion, location, and/or position of a user;
a display screen; and/or
any cables.

45. The device of any of Implementations 36-44, wherein said user input comprises a capacitive or resistive sensor.

46. The device of any of Implementations 36-45, wherein further comprising a microphone configured to receive audio from the user, and wherein said controller is configured to instruct the communication module to wirelessly transmit said received audio, and/or one or more signals generated based on said received audio, to the separate device.

47. The device of any of Implementations 36-46, further comprising a speaker, said speaker configured to emit audio based on one or more signals received by the communication module that are wirelessly transmitted from the separate device.

48. A device comprising one or more features of the foregoing description.

49. A method of monitoring and/or determining a physiological parameter of a user comprising one or more features of the foregoing description.

50. A self-contained adhesively and removably attached wearable electronic monitoring device comprising:
a housing comprising an interior, a top portion, and a bottom portion, said bottom portion configured to face toward a user during monitoring of one or more physiological parameters of the user, said bottom portion comprising a first opening, a second opening, and a third opening;
a diaphragm operably positioned proximate said first opening in said bottom portion, wherein, during monitoring, at least a portion of said diaphragm is configured to vibrate responsive to at least one of cardiac activity and lung activity of the user;
an audio transducer positioned within the interior of the housing and responsive to vibration of said diaphragm to output one or more transducer signals;
a motion sensor positioned within the interior of the housing, said motion sensor configured to generate one or more motion signals based on an orientation of the user;
a display proximate the top portion of the housing, the display including at least one display element responsive to an amount of health risk associated with the orientation of the user;
a first temperature sensor and a second temperature sensor positioned within the interior of the housing, each of said first and second temperature sensors configured to generate one or more first temperature signals responsive to detected thermal energy, said first temperature sensor operably positioned to be closer to the user during monitoring than the second temperature sensor;
a third temperature sensor and a fourth temperature sensor positioned within the interior of the housing, each of said third and fourth temperature sensors configured to generate one or more second temperature signals responsive to detected thermal energy, said third temperature sensor operably positioned to be closer to the user during monitoring than the fourth temperature sensor;
a thermally conductive element comprising a portion positioned between the third and fourth temperature sensors;
a first thermally conductive probe proximate the second opening of the housing and substantially aligned with the first temperature sensor;
a second thermally conductive probe proximate the third opening of the housing and substantially aligned with the third temperature sensor;
a user input proximate the top portion of the housing;
a communication module positioned within the interior of the housing and configured to allow the device to wirelessly communicate with a separate device; and
one or more hardware processors positioned within the interior of the housing;
wherein the one or more hardware processors are configured to:
   receive said one or more transducer signals;
   determine at least one of a cardiac measurement and a lung measurement responsive to said one or more transducer signals;
   receive said one or more motion signals;
   determine an orientation of the user relative to a surface responsive to said one or more motion signals;
   determine the amount of health risk responsive to the orientation of the user;
   change an appearance of the at least one display element responsive to the health risk;
   receive said first and second temperature signals;
   determine an indication of body temperature responsive to said first and second temperature signals;
   receive one or more user input signals responsive to said user input; and
   wirelessly output to the separate device through the communication module data indicative of determined parameters of the user.

51. The device of Implementation 50, wherein the one or more hardware processors are further configured to wirelessly output to the separate device through the communication module one or more communication signals based on said received one or more user input signals.

52. The device of any of Implementations 50-51, wherein the device comprises a first portion configured to be attached to the user and a second portion configured to removably secure to the first portion, the second portion comprising said housing.

53. A self-contained adhesively and removably attached wearable electronic monitoring device comprising:
a housing comprising an interior, a top portion, and a bottom portion, said bottom portion configured to face toward a user during monitoring of one or more physiological parameters of the user, said bottom portion comprising a first opening;
a diaphragm operably positioned proximate said first opening in said bottom portion, wherein, during monitoring, at least a portion of said diaphragm is configured to vibrate responsive to at least one of cardiac activity and lung activity of the user;
an audio transducer positioned within the interior of the housing and responsive to vibration of said diaphragm to output one or more transducer signals;
one or more other sensors or user inputs; and
one or more hardware processors positioned within the interior of the housing;
wherein the one or more hardware processors are configured to:
   receive said one or more transducer signals; and
   determine at least one of a cardiac measurement and a lung measurement responsive to said one or more transducer signals.

54. The device of Implementation 53, further comprising a communication module positioned within the interior of the housing and configured to allow the device to wirelessly communicate with a separate device, wherein the one or more hardware processors are further configured to wirelessly output to the separate device through the communication module the at least one of the cardiac measurement and the lung measurement.

55. The device of Implementation 53 or 54, further comprising a second audio transducer positioned within the interior of the housing responsive to at least one of vibration of the housing and sound waves external to the housing and output one or more second transducer signals, wherein the one or more hardware processors are further configured to:
receive said one or more second transducer signals; and
determine at least one of a corrected cardiac measurement and a corrected lung measurement responsive to said one or more transducer signals and said one or more second transducer signals.

56. The device of Implementation 55, wherein the one or one or more hardware processors are further configured to wirelessly output to the separate device through the communication module the at least one of the corrected cardiac measurement and the corrected lung measurement.

57. The device of any of Implementations 53-56, wherein said diaphragm is hermetically sealed to the bottom portion of the housing over said first opening.

58. The device of any of Implementations 53-57, wherein said diaphragm extends beyond an exterior surface of the bottom portion of the housing.

59. The device of any of Implementations 53-58, wherein said audio transducer is substantially aligned with an axis extending through a center of the diaphragm.

60. The device of any of Implementations 53-59, wherein the interior of the housing comprises an interior portion configured to isolate said vibration of said diaphragm.

61. The device of Implementation 60, wherein said interior portion is formed by at least a portion of said diaphragm and at least a portion of said bottom portion of the housing.

62. The device of any of Implementations 53-61, wherein the one or more other sensors or user inputs comprise:
a motion sensor positioned within the interior of the housing, said motion sensor configured to generate one or more motion signals based on an orientation of the user; and
the device further comprises:
   a display proximate the top portion of the housing, the display including at least one display element responsive to an amount of health risk associated with the orientation of the user;
wherein the one or more hardware processors are further configured to:
   receive said one or more motion signals;
   determine an orientation of the user relative to a surface responsive to said one or more motion signals;
   determine the amount of health risk responsive to the orientation of the user; and
   change an appearance of the at least one display element responsive to the health risk.

63. The device of any of Implementations 54-62, wherein the one or more other sensors or user inputs comprise:
a first temperature sensor and a second temperature sensor positioned within the interior of the housing, each of said first and second temperature sensors configured to generate one or more first temperature signals responsive to detected thermal energy, said first temperature sensor operably positioned to be closer to the user during monitoring than the second temperature sensor; and
a third temperature sensor and a fourth temperature sensor positioned within the interior of the housing, each of said third and fourth temperature sensors configured to generate one or more second temperature signals responsive to detected thermal energy, said third temperature sensor operably positioned to be closer to the user during monitoring than the fourth temperature sensor;
wherein the device further comprises:
   a second and a third opening in said bottom portion of the housing;
   a thermally conductive element comprising a portion positioned between the third and fourth temperature sensors;
   a first thermally conductive probe proximate the second opening of the housing and substantially aligned with the first temperature sensor;
   a second thermally conductive probe proximate the third opening of the housing and substantially aligned with the third temperature sensor;
wherein the one or more hardware processors are further configured to:
   receive said first and second temperature signals;
   determine an indication of body temperature responsive to said first and second temperature signals; and
   wirelessly output to the separate device through the communication module the determined indication of body temperature.

64. The device of any of Implementations 53-63, wherein the one or more other sensors or user inputs comprise:
a user input proximate the top portion of the housing;
wherein the one or more hardware processors are further configured to:
   receive one or more user input signals responsive to said user input; and
   wirelessly output to the separate device through the communication module one or more communication signals based on said received one or more user input signals.

65. A self-contained adhesively and removably attached wearable electronic monitoring device comprising:
a housing comprising an interior, a top portion, and a bottom portion, said bottom portion configured to face toward a user during monitoring of one or more physiological parameters of the user, said bottom portion comprising a first opening and a second opening;
a first temperature sensor and a second temperature sensor positioned within the interior of the housing, each of said first and second temperature sensors configured to generate one or more first temperature signals responsive to detected thermal energy, said first temperature sensor operably positioned to be closer to the user during monitoring than the second temperature sensor;
a third temperature sensor and a fourth temperature sensor positioned within the interior of the housing, each of said third and fourth temperature sensors configured to generate one or more second temperature signals responsive to detected thermal energy, said third temperature sensor operably positioned to be closer to the user during monitoring than the fourth temperature sensor;
a thermally conductive element comprising a portion positioned between the third and fourth temperature sensors;
a first thermally conductive probe proximate the second opening of the housing and substantially aligned with the first temperature sensor;
a second thermally conductive probe proximate the third opening of the housing and substantially aligned with the third temperature sensor;
one or more other sensors or user inputs; and
one or more hardware processors positioned within the interior of the housing;
wherein the one or more hardware processors are configured to:
   receive said first and second temperature signals; and
   determine an indication of body temperature responsive to said first and second temperature signals.

66. The device of Implementation 65, wherein the first and second temperature sensors are thermally insulated from one another by an air gap.

67. The device of Implementation 66, wherein:
the device further comprises a first circuit board and a second circuit board, the first and second circuit boards spaced from one another, the first circuit board positioned to be closer to the user during monitoring than the second circuit board;
said first temperature sensor is mounted to the first circuit board and the second temperature sensor is mounted on the second circuit board;
said third temperature sensor is mounted to the first circuit board and spaced from the first temperature sensor;
said fourth temperature sensor is mounted to the second circuit board and spaced from the second temperature sensor;
a distance between the first temperature sensor and the second circuit board at least partially defines said air gap; and
said portion of the thermally conductive element is positioned between a portion of the first circuit board that is adjacent to the third temperature sensor and a portion of the second circuit board that is adjacent to the fourth temperature sensor.

68. The device of Implementation 67, wherein:
said first circuit board comprises a first surface and a second surface;
said second circuit board comprises a first surface and a second surface;
said first surface of the first circuit board faces toward the second surface of the second circuit board;
said first and third temperature sensors are mounted on the first surface of the first circuit board; and
said second and fourth temperature sensors are mounted on the first surface of the second circuit board.

69. The device of any of Implementations 67-68, wherein:
said first circuit board comprises an opening proximate the third temperature sensor;
said second circuit board comprises an opening proximate the fourth temperature sensor;
a first end of the thermally conductive element is positioned within said opening of the first circuit board;
a second end of the thermally conductive element is positioned within said opening of the second circuit board;
the first circuit board comprises a thermally conductive material configured to allow thermal energy to pass from the first end of the thermally conductive element to the third temperature sensor; and
the second circuit board comprises a thermally conductive material configured to allow thermal energy to pass from the second end of the thermally conductive element to the fourth temperature sensor.

70. The device of any of Implementations 65-69, wherein said first and second temperature sensors are substantially aligned with one another and wherein said third and fourth temperature sensors are substantially aligned with one another.

71. The device of any of Implementations 67-70, wherein the first and second circuit boards are arranged to be substantially parallel to one another.

72. The device of any of Implementations 65-71, wherein the device comprises a first portion configured to be attached to the user and a second portion configured to removably secure to the first portion, the second portion comprising said housing.

73. The device of Implementation 72, wherein the first portion comprises a frame and a substrate coupled to the frame, the substrate configured to secure to the user.

74. The device of Implementation 73, wherein the first and second thermally conductive probes contact the substrate and the substrate is positioned between the user and the first and second thermally conductive probes during monitoring.

75. The device of any of Implementations 65-74, wherein the first and second thermally conductive probes are configured to transmit thermally energy of the user toward the first and third temperature sensors.

76. The device of any of Implementations 65-75, wherein the first and second thermally conductive probes extend through said first and second openings in the bottom portion of the housing and beyond an exterior surface of the bottom portion of the housing.

77. The device of any of Implementations 65-76, wherein:
the device further comprises a communication module positioned within the interior of the housing and configured to allow the device to wirelessly communicate with a separate device; and
the one or more hardware processors are further configured to wirelessly output to the separate device through the communication module the determined indication of body temperature.

78. The device of any of Implementations 65-77, wherein the device further comprises:
a third opening in said bottom portion of the housing; and
a diaphragm operably positioned proximate said third opening in said bottom portion, wherein, during monitoring, at least a portion of said diaphragm is configured to vibrate responsive to at least one of cardiac activity and lung activity of the user; and
wherein the one or more sensors or user inputs comprise:
   an audio transducer positioned within the interior of the housing and responsive to vibration of said diaphragm to output one or more transducer signals;
wherein the one or more hardware processors are further configured to:
   receive said one or more transducer signals;
   determine at least one of a cardiac measurement and a lung measurement responsive to said one or more transducer signals.

79. The device of any of Implementations 65-78, wherein the one or more other sensors or user inputs comprise:
a motion sensor positioned within the interior of the housing, said motion sensor configured to generate one or more motion signals based on an orientation of the user; and
the device further comprises:
   a display proximate the top portion of the housing, the display including at least one display element responsive to an amount of health risk associated with the orientation of the user;
wherein the one or more hardware processors are further configured to:
   receive said one or more motion signals;
   determine an orientation of the user relative to a surface responsive to said one or more motion signals;
   determine the amount of health risk responsive to the orientation of the user; and
   change an appearance of the at least one display element responsive to the health risk.

80. The device of any of Implementations 65-79, wherein the one or more other sensors or user inputs comprise:
a user input proximate the top portion of the housing; and
wherein the device further comprises:
   a communication module positioned within the interior of the housing and configured to allow the device to wirelessly communicate with a separate device;
wherein the one or more hardware processors are further configured to:
   receive one or more user input signals responsive to said user input; and
   wirelessly output to the separate device through the communication module one or more communication signals based on said received one or more user input signals.

81. A self-contained adhesively and removably attached wearable electronic monitoring device comprising:
a housing comprising an interior, a top portion, and a bottom portion, said bottom portion configured to face toward a user during monitoring of one or more physiological parameters of the user;
a user input proximate the top portion of the housing;
a communication module positioned within the interior of the housing and configured to allow the device to wirelessly communicate with a separate device;
one or more other sensors or user inputs; and
one or more hardware processors positioned within the interior of the housing;
wherein the one or more hardware processors are configured to:
   receive one or more user input signals responsive to said user input; and
   wirelessly output to the separate device through the communication module one or more communication signals based on said received one or more user input signals.

82. The device of Implementation 81, wherein:
the bottom portion of the housing comprises a first opening;
the device further comprises a diaphragm operably positioned proximate said first opening in said bottom portion, wherein, during monitoring, at least a portion of said diaphragm is configured to vibrate responsive to at least one of cardiac activity and lung activity of the user; and
the one or more other sensors or user inputs comprise an audio transducer positioned within the interior of the housing and responsive to vibration of said diaphragm to output one or more transducer signals;
wherein the one or more hardware processors are further configured to:
   receive said one or more transducer signals;
   determine at least one of a cardiac measurement and a lung measurement responsive to said one or more transducer signals.

83. The device of any of Implementations 81-82, wherein the one or more other sensors or user inputs comprise:
a motion sensor positioned within the interior of the housing, said motion sensor configured to generate one or more motion signals based on an orientation of the user; and
the device further comprises:
   a display proximate the top portion of the housing, the display including at least one display element responsive to an amount of health risk associated with the orientation of the user;
wherein the one or more hardware processors are further configured to:
   receive said one or more motion signals;
   determine an orientation of the user relative to a surface responsive to said one or more motion signals;
   determine the amount of health risk responsive to the orientation of the user; and
   change an appearance of the at least one display element responsive to the health risk.

84. The device of any of Implementations 81-83, wherein the one or more other sensors or user inputs comprise:
a first temperature sensor and a second temperature sensor positioned within the interior of the housing, each of said first and second temperature sensors configured to generate one or more first temperature signals responsive to detected thermal energy, said first temperature sensor operably positioned to be closer to the user during monitoring than the second temperature sensor; and
a third temperature sensor and a fourth temperature sensor positioned within the interior of the housing, each of said third and fourth temperature sensors configured to generate one or more second temperature signals responsive to detected thermal energy, said third temperature sensor operably positioned to be closer to the user during monitoring than the fourth temperature sensor;
wherein the device further comprises:
   a second and a third opening in said bottom portion of the housing;
   a thermally conductive element comprising a portion positioned between the third and fourth temperature sensors;
   a first thermally conductive probe proximate the second opening of the housing and substantially aligned with the first temperature sensor;
   a second thermally conductive probe proximate the third opening of the housing and substantially aligned with the third temperature sensor;
wherein the one or more hardware processors are further configured to:
   receive said first and second temperature signals;
   determine an indication of body temperature responsive to said first and second temperature signals; and
   wirelessly output to the separate device through the communication module the determined indication of body temperature.

85. The device of any of Implementations 81-84, wherein the one or more hardware processors are further configured to wirelessly output to the separate device through the communication module data indicative of determined parameters of the user.

86. The device of any of Implementations 50-85, further comprising a plurality of cables and corresponding external ECG electrodes, said external ECG electrodes configured to attach to the user and output one or more signals responsive to the user's cardiac electrical activity;
wherein the one or more hardware processors are further configured to:
   receive said one or more signals from said external ECG electrodes responsive to the user's cardiac electrical activity; and
   determine an ECG of the user responsive to said one or more signals.

87. The device of any of Implementations 50-86, further comprising one or more internal ECG electrodes, said one or more internal ECG electrodes configured to output one or more signals responsive to the user's cardiac electrical activity;
wherein the one or more hardware processors are further configured to:
   receive said one or more signals from said internal ECG electrodes responsive to the user's cardiac electrical activity; and
   determine an ECG of the user responsive to said one or more signals.

### Additional Considerations and Terminology

Although this invention has been disclosed in the context of certain preferred implementations, it should be understood that certain advantages, features and aspects of the systems, devices, and methods may be realized in a variety of other implementations. Additionally, it is contemplated that various aspects and features described herein can be practiced separately, combined together, or substituted for one another, and that a variety of combination and subcombinations of the features and aspects can be made and still fall within the scope of the invention. Furthermore, the systems and devices described above need not include all of the modules and functions described in the preferred implementations.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more implementations necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the implementation, certain acts, events, or functions of any of the processes or algorithms described herein can be performed in a different sequence, can be added, merged, or left out altogether (for example, not all described operations or events are necessary for the practice of the algorithm). Moreover, in certain implementations, operations or events can be performed concurrently, e.g., through multi-threaded processing, interrupt processing, or multiple processors or processor cores or on other parallel architectures, rather than sequentially.

Various illustrative logical blocks, modules, routines, and algorithm steps that may be described in connection with the disclosure herein can be implemented as electronic hardware (e.g., ASICs or FPGA devices), computer software that runs on general purpose computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the disclosure herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. A processor can include an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A self-contained adhesively and removably attached wearable electronic monitoring device comprising:
a housing comprising an interior, a top portion, and a bottom portion, said bottom portion configured to face toward a user during monitoring of one or more physiological parameters of the user;
a motion sensor positioned within an interior of the housing, said motion sensor configured to generate one or more signals based on an orientation of the user;
a display proximate the top portion of the housing, the display including at least one display element responsive to an amount of health risk associated with the orientation of the user;
one or more other sensors or user inputs; and
one or more hardware processors positioned within the interior of the housing;
wherein the one or more hardware processors are configured to:
receive said one or more motion signals;
determine the orientation of the user relative to a surface responsive to said one or more motion signals;
determine the amount of health risk responsive to the orientation of the user; and
change an appearance of the at least one display element responsive to the health risk.

2. The device of claim 1, wherein said health risk is at least partially dependent upon an amount of time the user is in the orientation.

3. The device of claim 2, wherein said amount of time is not consecutive.

4. The device of claim 1, wherein the one or more hardware processors are further configured to:
for each respective orientation of a plurality of orientations of the user with respect to the surface:
increase a value of a timer associated with the respective orientation when the user is in the respective orientation;
decrease the value of the timer when the user is not in the respective orientation;
determine the amount of health risk for the respective orientation based at least in part on the value of the timer; and
for each respective one of a plurality of display elements of the display:
change an appearance of the respective one of the plurality of display elements based at least in part on the health risk associated with one of said plurality of orientations.

5. The device of claim 4, wherein:
a first one of said plurality of orientations is associated with a left side orientation of the user with respect to the surface;
a second one of said plurality of orientations is associated with a right side orientation of the user with respect to the surface; and
a third one of said plurality of orientations is associated with a supine orientation of the user with respect to the surface.

6. The device of claim 4, wherein, for each respective one of the plurality of display elements of the display, the one or more hardware processors are further configured to:
cause the appearance of the respective one of the plurality of display elements to have a first color when the health risk is greater than or equal to a threshold; and
cause the appearance of the respective one of the plurality of portions to have a second color when the health risk is below the threshold, said second color being different than said first color.

7. The device of claim 5, wherein said plurality of orientations further comprises a plurality of orientations between said first one and said second one of said plurality of orientations including said third one.

8. The device of claim 1, wherein said health risk is associated with a combination of a plurality of factors.

9. The device of claim 8, wherein at least one of said factors is a physiological parameter of the user.

10. The device of claim 1, wherein said display comprises an arch shape.

11. The device of claim 4, wherein:
said display comprises a border having at least a first edge and a second edge; and
each of said plurality of display elements of the display comprises a line or a region extending between the first and second edges of the border.

12. The device of claim 1, wherein said display only illustrates said health risk and does not include any other information.

13. The device of claim 1, wherein the device comprises a first portion configured to be attached to the user and a second portion configured to removably secure to the first portion, the second portion comprising said housing.

14. The device of claim 13, wherein the first portion comprises a frame and a substrate coupled to the frame, the substrate configured to be attached to the user.

15. The device of claim 1, wherein:
the bottom portion of the housing comprises a first opening;
the device further comprises:
a diaphragm operably positioned proximate said first opening in said bottom portion, wherein, during monitoring, at least a portion of said diaphragm is configured to vibrate responsive to at least one of cardiac activity and lung activity of the user;
a communication module positioned within the interior of the housing and configured to allow the device to wirelessly communicate with a separate device; and
the one or more other sensors or user inputs comprise an audio transducer positioned within the interior of the housing and responsive to vibration of said diaphragm to output one or more transducer signals;
wherein the one or more hardware processors are further configured to:
receive said one or more transducer signals;
determine at least one of a cardiac measurement and a lung measurement responsive to said one or more transducer signals; and
wirelessly output to the separate device through the communication module data indicative of determined parameters of the user.

16. The device of claim 1, wherein the one or more other sensors or user inputs comprise:
a first temperature sensor and a second temperature sensor positioned within the interior of the housing, each of said first and second temperature sensors configured to generate one or more first temperature signals responsive to detected thermal energy, said first temperature sensor operably positioned to be closer to the user during monitoring than the second temperature sensor; and
a third temperature sensor and a fourth temperature sensor positioned within the interior of the housing, each of said third and fourth temperature sensors configured to generate one or more second temperature signals responsive to detected thermal energy, said third temperature sensor operably positioned to be closer to the user during monitoring than the fourth temperature sensor;
wherein the device further comprises:
a second and a third opening in said bottom portion of the housing;
a thermally conductive element comprising a portion positioned between the third and fourth temperature sensors;
a first thermally conductive probe proximate the second opening of the housing and substantially aligned with the first temperature sensor;
a second thermally conductive probe proximate the third opening of the housing and substantially aligned with the third temperature sensor;
a communication module positioned within the interior of the housing and configured to allow the device to wirelessly communicate with a separate device;
wherein the one or more hardware processors are further configured to:
receive said first and second temperature signals;
determine an indication of body temperature responsive to said first and second temperature signals; and
wirelessly output to the separate device through the communication module the determined indication of body temperature.

17. The device of claim 1, wherein the one or more other sensors or user inputs comprise:
a user input proximate the top portion of the housing; and
wherein the device further comprises:
a communication module positioned within the interior of the housing and configured to allow the device to wirelessly communicate with a separate device;
wherein the one or more hardware processors are further configured to:
receive one or more user input signals responsive to said user input; and
wirelessly output to the separate device through the communication module one or more communication signals based on said received one or more user input signals.

18. The device of claim 1, further comprising a plurality of cables and corresponding external ECG electrodes, said external ECG electrodes configured to attach to the user and output one or more signals responsive to the user's cardiac electrical activity;
wherein the one or more hardware processors are further configured to:
receive said one or more signals from said external ECG electrodes responsive to the user's cardiac electrical activity; and
determine an ECG of the user responsive to said one or more signals.

19. The device of claim 1, further comprising one or more internal ECG electrodes, said one or more internal ECG electrodes configured to output one or more signals responsive to the user's cardiac electrical activity;
wherein the one or more hardware processors are further configured to:
receive said one or more signals from said internal ECG electrodes responsive to the user's cardiac electrical activity; and
determine an ECG of the user responsive to said one or more signals.
